(19) United States
(12) Patent Application Publication
(10) Pub. No.: US 2026/0108496 A1
(43) Pub. Date: Apr. 23, 2026

(54) METHODS FOR REPROGRAMING EXHAUSTED T CELLS AND BOOSTING IMMUNE CHECKPOINT BLOCKADE THERAPY FOR CANCER

(71) Applicant: Ohio State Innovation Foundation, Columbus, OH (US)

(72) Inventor: Hazem GHONEIM, Dublin, OH (US)

(21) Appl. No.: 19/111,688

(22) PCT Filed: Sep. 14, 2023
(86) PCT No.: PCT/US2023/074259

§ 371 Date: Mar. 13, 2025

Related U.S. Application Data
(60) Provisional application No. 63/406,415, filed on Sep. 14, 2022.

(51) Int. Cl. A61K 31/421; A61K 31/167; A61K 31/375; A61K 31/42; A61K 31/4375; A61K 31/444; A61K 31/4709; A61K 31/4985; A61K 31/519; A61K 31/7105; A61K 38/46; A61P 35/00

(52) U.S. Cl. CPC A61K 31/421; A61K 31/167; A61K 31/375; A61K 31/42; A61K 31/4375; A61K 31/444; A61K 31/4709; A61K 31/4985; A61K 31/519; A61K 31/7105; A61K 38/465; A61P 35/00

(57) ABSTRACT

Disclosed are methods and compositions related to reviving the functionality for exhausted T cells and methods of improving immunotherapy by inhibiting T cell exhaustion. In one aspect, the methods and compositions described herein comprise inhibitors of TGFβR1 and/or BMP4 proteins or agonists to rescue the functional phenotype of exhausted T cells.

### I. CROSS REFERENCE TO RELATED APPLICATIONS

This application claims the benefit of U.S. Provisional Application No. 63/406,415, filed on Sep. 14, 2022, which is incorporated herein by reference in its entirety.

### II. REFERENCE TO SEQUENCE

A Sequence Listing conforming to the rules of WIPO Standard ST.26 is hereby incorporated by reference. Said Sequence Listing has been filed as an electronic document via Patent Center in ASCII format encoded as XML. The electronic document, created on Sep. 14, 2023, is entitled “103361-363WO1.xml”, and is 2810 bytes in size.

### III. BACKGROUND

Cytotoxic CD8 T lymphocytes are an important defense against tumors or virus-infected cells; progressively lose their killing function and become exhausted during cancer or chronic virus infections. T cell exhaustion remains a major challenge to T cell immunotherapy. In some cases, this barrier can be surmounted with immune checkpoint blockade (ICB), which rejuvenates partially-exhausted T cells by blocking signals from inhibitory receptors. Despite the success of ICB in treating some previously refractory cancers, many patients remain nonresponsive. As such, what is needed are new treatments and methods that target molecular barriers for rejuvenation of terminally-exhausted T cells that remain refractory to ICB therapy.

### IV. SUMMARY

Disclosed are methods and compositions related to reviving the functionality for exhausted T cells and methods of improving immunotherapy by inhibiting T cell exhaustion.

In one aspect, disclosed herein are methods of rescuing the functional phenotype of exhausted T cells in a subject in need thereof comprising administering to the subject a 1) transforming growth factor-β receptor 1 (TGFβR1) inhibitor (such as, for example, RepSox, SB525334, GW788388, Vactosertib, SD-208, Galunisertib, and/or LY3200882 or a vector encoding a CRISPR/Cas9 endonuclease integration system comprising a guide RNA (gRNA) that targets TGFβR1 gene) and 2) a bone morphogenic protein 4 (BMP4), BMP6, BMP10 protein, or a BMP4, BMP6, or BMP10 agonist (such as, for example, the BMP4 agonists SB4, SJ000063181, SJ000291942, and/or SJ000370178). In one aspect, the method further comprises the administration of an antioxidant, including, but not limited to vitamin C.

In one aspect, disclosed herein are methods of rescuing the functional phenotype of exhausted T cells in a subject in need thereof comprising administering to the subject a vector (such as, for example, an adeno-associated virus (AAV) vector including, but not limited to AAV1, AAV2, AAV3, AAV4, AAV5, AAV6, AAV7, AAV8, or AAV9) encoding clustered regularly interspaced short palindromic repeat (CRISPR)/CRISPR-associated 9 (Cas9) endonuclease integration system wherein the Cas9 endonuclease complexed with a guide RNA (gRNA) that targets TGFβR1 gene; and wherein expression of the CRISPR/Cas9 endonuclease integration systems excises all or a functional fragment of the TGFβR1. In one aspect, the expression of the Cas9 endonuclease is operatively linked to a T cell specific promoter, inducible promoter, or constitutive promoter).

Also disclosed herein are cancer or infectious disease treatment regimens comprising 1) one or more checkpoint inhibitors (including, but are not limited to antibodies that block PD-1 (such as, for example, Nivolumab (BMS-936558 or MDX1106), pembrolizumab, CT-011, MK-3475), PD-L1 (such as, for example, alezolizumab, avelumab, durvalumab, MDX-1105 (BMS-936559). MPDL3280A, or MSB0010718C), PD-L2 (such as, for example, rHIgM12B7). CTLA-4 (such as, for example, Ipilimumab (MDX-010), Tremelimumab (CP-675,206)), IDO, B7-H3 (such as, for example, MGA271, MGD009, omburtamab), B7-H4, T cell immunoreceptor with Ig and ITIM domains (TIGIT)(such as, for example BMS-986207, OMP-313M32, MK-7684, AB-154, ASP-8374, MTIG7192A, or PVSRIPO). CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep), 2) one or more transforming growth factor-β receptor 1 (TGFβR1) inhibitors (such as, for example, RepSox, SB525334, GW788388, Vactosertib, SD-208, Galunisertib, and/or LY3200882 or a vector encoding a CRISPR/Cas9 endonuclease integration system comprising a guide RNA (gRNA) that targets TGFβR1 gene) and 3) one or more bone morphogenic protein 4 (BMP4), BMP6, or BMP10 protein, and/or a BMP4. BMP6, or BMP10 agonists (such as, for example, the BMP4 agonists SB4, SJ000063181, SJ000291942, and/or SJ000370178). In one aspect, the regimen further comprises an antioxidant, including, but not limited to vitamin C.

In one aspect, disclosed herein are methods of treating, inhibiting, reducing, decreasing, ameliorating, and/or preventing a cancer and/or metastasis in a subject or methods of treating, inhibiting, reducing, decreasing, ameliorating, and/or preventing an infectious disease (such as, for example a viral infection, bacterial infection, parasitic infection, or fungal infection) in a subject, the method comprising administering to the subject the treatment regimen of any preceding aspect. For example, disclosed herein are methods of treating, inhibiting, reducing, decreasing, ameliorating, and/or preventing a cancer and/or metastasis in a subject or methods of treating, inhibiting, reducing, decreasing, ameliorating, and/or preventing an infectious disease (such as, for example a viral infection, bacterial infection, parasitic infection, or fungal infection) in a subject, the method comprising administering to the subject 1) one or more checkpoint inhibitors (including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, B1754111, Sym022, FS118, MGD013, and Immutep), 2) one or more transforming growth factor-β receptor 1 (TGFβR1) inhibitors (such as, for example, RepSox, SB525334, GW788388, Vactosertib. SD-208, Galunisertib, and/or LY3200882 or a vector encoding a CRISPR/Cas9 endonuclease integration system comprising a guide RNA (gRNA) that targets TGFβR1 gene) and 3) one or more bone morphogenic protein 4 (BMP4), BMP6, or BMP10 protein, and/or a BMP4, BMP6, or BMP10 agonists (such as, for example, the BMP4 agonists SB4, SJ000063181, SJ000291942, and/or SJ000370178). In one aspect, the method further comprises administering to the subject an antioxidant, including, but not limited to vitamin C.

Also disclosed herein are methods of increasing the efficacy of or reducing resistance to an immune checkpoint blockade in a subject receiving treatment with an immune checkpoint inhibitor comprising administering to the subject 1) one or more transforming growth factor-β receptor 1 (TGFβR1) inhibitors (such as, for example, RepSox, SB525334, GW788388, Vactosertib, SD-208. Galunisertib, and/or LY3200882 of a vector encoding a CRISPR/Cas9 endonuclease integration system comprising a guide RNA (gRNA) that targets TGFβR1 gene) and 2) one or more bone morphogenic protein 4 (BMP4), BMP6, or BMP10 protein, and/or a BMP4, BMP6, or BMP10 agonists (such as, for example, the BMP4 agonists SB4, SJ000063181, SJ000291942, and/or SJ000370178).

In one aspect, disclosed herein are methods of increasing the efficacy of or reducing resistance to an immune checkpoint blockade in a subject of any preceding aspect, wherein the immune checkpoint inhibitor includes, but is not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep).

### V. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A and 1B show de novo DNA methylation is essential for establishing CD8 T cell exhaustion. FIG. 1A shows a representative FACS plots and bar graphs showing the production levels of IFNγ and IL-2 in CD8 T cells after ex vivo gp33 peptide-stimulation of splenocytes isolated from chronically LCMV-infected WT and Dnmt3a cKO mice. FIG. 1B shows a representative FACS plots and bar graph showing the expression levels of Tcf1 and Ki67 in gp33-specific CD8 T cells from chronically infected WT and cKO mice. N≥4 mice per group, 3 independent experiments. * P-value <0.05. *** P-value <0.001 (Mann-Whitney U test). Error bars indicate SEM.

FIGS. 2A and 2B show de novo DNA methylation enforces the silencing of effector and stemness programs in exhausted T cells. FIG. 2A shows snapshots of the WGBS datasets (GSE99450) showing de novo DMRs in naïve, effector, and chronically stimulated antigen-specific WT or Dnmt3a cKO CD8 T cells in the 1fng and Tbx21 loci, and 2B in the TcF7 locus. The ratio of blue to red indicates the % of unmethylated versus methylated reads, respectively.

FIGS. 3A, 3B, and 3C show TOX remodels the open chromatin landscape of CD8 T cells toward the exhaustion. FIG. 3A shows the numbers of OCRs that were gained or lost in Tox KO versus WT antigen-specific CD8 T cells on days 8 and 13 post-chronic LCMV infection. FIG. 3B shows representative epigenetic analysis and (3C) the average DNA methylation levels of the Tox DMR in naïve, memory, and exhausted CD8 T cells. n=3-4 samples per group. *** P-value<0.001 (Unpaired t test with Welch's correction), Error bars indicate SEM.

FIGS. 4A, 4B, and 4C show we novo DNA methylation shapes the open chromatin landscape of exhausted CD8 T cells. FIG. 4A shows dot plot showing the ATAC-seq peak signal intensity at the detected open chromatin peaks and the corresponding ratios of DNA methylation levels in exhausted WT CD8 T cells. FIG. 4B shows a bar graph indicating the numbers of OCRs that were gained (green) or lost (red) in naïve or antigen-specific Dnmt3a cKO versus WT CD8 T cells on days 8 and 35 post-chronic LCMV infection. FIG. 4C shows 3-D tri-surface plot tracking the Log₂ fold-change in chromatin accessibility of the detected OCRs (x-axis; adjusted P<0.05) at both day 8 (y-axis) and day 35 (z-axis) p.i in Dnmt3a cKO versus WT antigen-specific CD8 T cells. Color legend represents the Log₂ fold-changes at day 35 p.i (z-axis).

FIGS. 5A, 5B, and 5C show TGFβ signaling is the most significant pathway that orchestrates epigenetic and transcriptional changes in exhausted CD8 T cells from mice or humans. FIGS. 5A and 5B show comparative Ingenuity Pathway Analysis of the top upstream regulators of changes in DNA methylation (datasets 1 & 3 from GSE99450), chromatin accessibility (datasets 5 & 10 from GSE89308), or transcriptional programs (dataset 6 from GSE89307; dataset 7 from GSE123235; datasets 8 & 9 from BioProject: PRJNA497086; dataset 11 from GSE140430) in exhausted T cells during chronic LCMV infection or solid tumors in mice and humans. FIG. 5C shows a volcano plot of the Reactome Pathway Enrichment analysis for Dnmt3a-target genes that retained chromatin accessibility or transcriptional activity in Dnmt3a-cKO versus WT CD8 T cells during chronic LCMV infection.

FIGS. 6A, 6B, 6C, 6D, and 6E show negative regulators of Smad2/3-TGFβ signaling are progressively downregulated in CD8 T cells during the progression to full-exhaustion. Heatmaps showing RNA expression levels in: FIG. 6A shows partially- versus fully-exhausted CD8 T cells during chronic LCMV infection (GSE132110) or B16 melanoma tumors (GSE122713); FIG. 6B shows partially- versus fully-exhausted TILs from human kidney tumors (GSE140430); and FIGS. 6C, 6D, and 6E show RNA-levels in partially-exhausted, CD101− Tim-3+ Transitory exhausted, and CD101+ Tim-3+ fully-exhausted subsets during chronic LCMV infection (BioProject: PRJNA497086).

FIG. 7 shows regulators of TGFβ/BMP-signaling are epigenetically silenced in fully-exhausted CD8 T cells. Snapshots of the WGBS and ATAC-seq datasets showing the DNA methylation and open chromatin peaks in naïve (grey), and chronically stimulated, antigen-specific WT (red) or Dnmt3a cKO CD8 T cells from (green), or partially- versus fully-exhausted CD8 T cells (GSE132110). The ratio of blue to red indicates the % of unmethylated versus methylated reads, respectively.

FIGS. 8A, 8B, 8C, and 8D show therapeutic blockade of TGFβ1-signaling inhibits functional exhaustion of persistently stimulated CD8 T cells in vitro. Bar graphs showing the expression levels of 8A) IFNγ, 8B) TNFα, and 8D) T-bet in anti-tumor P14 CD8 T cells during repeated co-culture with gp33-expressing CT2A glioma cells. FIG. 8C shows a bar graph showing the percent of dead tumor cells during in-vitro co-culture. Treatment of co-cultured tumor and P14 cells was initiated on day 6 post-co-culture as indicated (n 4 wells per treatment, 3 independent experiments). ** P-value <0.01, *** <0.001, **** <0.0001 (ANOVA-Dunnett's test compared to Day 6), #P-value <0.01 (ANOVA, Dunnett's test compared to Vehicle group). Error bars indicate SEM.

FIGS. 9A, 9B, and 9C show therapeutic blockade of TGFβ1-signaling plus augmenting BMP-signals promotes survival of persistently stimulated CD8 T cells in vitro. FIG. 9A shows numbers of P14 cells during prolonged co-culture with CT2A-gp33 tumor cells under the same treatment conditions in FIG. 8. FIG. 9B shows longitudinal analysis of the expression levels of CD62L and 9C) PD-1 on persistently stimulated P14 cells. (n 4 wells per treatment, 3 independent experiments), *P<0.05, *** <0.001, **** <0.0001 (ANOVA-Dunnett's test compared to Vehicle group). Error bars indicate SEM

FIGS. 10A, 10B, and 10C show that decitabine treatment potentiates the response of exhausted anti-tumor CD8 T cells to ICB therapy. FIG. 10A shows sequential decitabine (DAC) and anti-PD-L1 treatments of TRAMP-C2 tumor-bearing mice starting at >30 days post-tumor implantation. FIG. 10B shows representative FACS plots and bar graphs of Ki67 levels among PD-1^{high} or tumor antigen-specific (Spas1+) CD8 TILs after mono anti-PD-L1 or sequential treatments. FIG. 10C shows tumor growth in mice receiving mono anti-PD-L1 (red) or sequential treatments (blue). * P-value <0.05 (Mann-Whitney U test). Error bars indicate SEM.

FIGS. 11A and 11B show blocking de novo DNA methylation skews the open chromatin landscape of persistently stimulated CD8 T cells toward the functional memory state. FIG. 11A shows multiple-line graph showing chromatin accessibility changes in effector (red) and (11B) stemness-associated (green) programs in naïve, effector, exhausted WT, chronically stimulated Dnmt3a-deficient, and memory CD8 T cells.

FIGS. 12A, 12B, 12C, and 12D show blocking de novo DNA methylation in CD8 T cells preserves the rejuvenation potential after stopping ICB treatment. FIG. 12A shows experimental setup of anti-PD-L1 treatment in chronically infected WT and Dnmt3a cKO mice. FIG. 12B shows longitudinal tracking of gp33-specific CD8 T cell quantity in the peripheral blood of treated mice after stopping anti-PD-L1 treatment. FIGS. 12C and 12D show representative FACS plots of Ki67 levels and fold change of proliferating cells among LCMV-specific CD8 T cells 2 days or 1 month after anti-PD-L1 treatment. n>4 mice/group, 3 independent exps. *P<0.05, **P<0.01, ***P<0.001 (Mann-Whitney U test). Error bars indicate SEM.

FIGS. 13A, 13B, 13C, 13D, 13E, 13F, 13G, 13H, 13I, 13J, 13K, 13L, 13M, 13N, 13O, and 13P show prolonged post-effector TGFβ1 signaling accelerates severe exhaustion of chronically stimulated human CD8+ T cells. FIG. 13A shows a schematic for ex vivo stimulation of human cord blood mononuclear cells (CBMCs) under acute (day 0-7) or chronic (day 0-28) TCR stimulation. “Weak” TCR=soluble anti-CD3 stimulation; “Strong” TCR=plate-bound anti-CD3/CD28 stimulation (day 0-7), then repeated plate-bound anti-CD3 stimulation of FACS-purified CD8+ T cells (day 7-28). FIG. 13B shows a longitudinal tracking of human CD8+ T cell expansion and (13C) frequency of IFNγ-producing CD8+ T cells under acute or chronic “weak” TCR stimulation. FIG. 13D shows a summary bar graph showing GZMB expression levels in CD8+ T cells on day 28. FIG. 13E shows a longitudinal tracking of frequency of IFNγ and CD107a co-producing CD8+ T cells for acute versus chronic “weak”, or (13F) “strong” TCR stimulation from day 0-28. FIG. 13G shows a summary bar graphs showing expression levels of CD39 and (13H) LAG3 in CD8+ T cells under “strong” TCR stimulation at day 28. FIG. 13I shows a multi-IPA analysis for upstream regulators of distinct epigenetic DNA methylation (red), chromatin accessibility (blue), or transcriptional (green) programs in exhausted versus memory or terminally exhausted versus partially exhausted subsets of CD8+ T cells during chronic infection or cancer. FIG. 13J shows a schematic for chronic TCR stimulation of human CBMCs in the presence of TGFβ1 from day 7-28. FIG. 13K shows summary bar graphs showing frequencies of IFNγ and CD107a co-producing and (13L) expression levels of T-bet in IFNγ+ CD8+ T cells after PMA/Ionomycin stimulation on day 7, 14, 21 and 28 of chronic “weak” TCR. FIG. 13M shows a summary bar graphs showing frequencies of IFNγ and CD107a co-producing or (13N) IFNγ and TNFα co-producing CD8+ T cells, and expression levels of (13O) Perforin and (13P) CD101 in CD8+ T cells after PMA/Ionomycin stimulation on day 28 of “strong” TCR. N≥4 per group from 2-3 independent experiments. Comparisons were made using the Mann-Whitney U test. * p-value <0.05, ** p<0.01, *** p<0.001 relative to Ch.TCR group or as indicated. Error bars indicate mean SEM.

FIGS. 14A, 14B, 14C, 14D, 14E, 14F, 14G, 14H, 14I, 14J, 14K, 14L, 14M, 14N, 14O, 14P, 14Q, and 14R show chronic TGFβ1 and TCR signals establish a stable dysfunctional program in CD8+ T cells. FIG. 14A shows a schematic for the resting phase of acutely or chronically stimulated human CD8+ T cells under homeostatic conditions. Dysf=Chronic TCR plus TGFβ1. FIG. 14B shows longitudinal tracking of frequencies of IFNγ-producing CD8+ T cells during resting for 14 days (day 28-42) for the conditions in FIG. 14A. FIG. 14C shows summary bar graphs showing frequencies of polyfunctional (IFNγ+ TNFα+ CD107a+) CD8+ T cells after PMA/Ionomycin stimulation on day 42. FIG. 14D shows summary bar graphs showing expression levels of IFNγ and (14E) T-bet in IFNγ+ CD8+ T cells, (14F) frequencies of TNFα-producing or (14G) CD107a+ CD8+ T cells, and (14H) expression levels of TCF1 or (i) IL-7R on CD8+ T cells after PMA/Ionomycin stimulation on day 42. FIG. 14J shows summary bar graph for frequencies of dead CD8+ T cells on day 42, and (14K) frequencies of divided CD8+ T cells with 23 proliferation cycles by CFSE staining. FIG. 14L shows a schematic for the resting phase from acute or chronic “strong” TCR plus TGFβ1 of human CD8+ T cells under homeostatic conditions. FIG. 14M shows summary bar graphs showing frequencies of IFNγ and CD107a co- producing CD8+ T cells, and expression levels of (14N) CD107a, (o) Perform, (p) CD11a, (q) CD101, and (r) CD103 in CD8+ T cells after PMA/Ionomycin stimulation on day 35. N≥4 per group from 2-3 independent experiments. Comparisons were made using the Mann-Whitney U test (panels m-r) or unpaired t test with Welch's correction (panels b-k). * p-value <0.05,** p<0.01, *** p<0.001, **** p<0.0001 relative to Dysf. condition (panels c-k), Ac.TCR (panels m-r), or as indicated. Error bars are the mean±SEM.

FIGS. 15A, 15B, 15C, 15D, 15E, 15F, 15G, 15H, 15I, 15J, 15K, 15L, and 15M show BMP4 agonist treatment reverses exhaustion features and enhances survival of human CD8+ T cells under chronic strong TCR-stimulation. FIG. 15A shows a schematic for ex vivo stimulation of human cord blood mononuclear cells (CBMCs) under chronic (day 0-21) “strong” TCR stimulation and treatment with TGFβ1 or BMP4 agonist from day 7-21. FIG. 15B shows summary bar graphs showing frequencies of total IFNγ-producing, (15c) IFNγ and CD107a co-producing CD8+ T cells, or (15d) expression levels of CD107a and (15e) CD39 in IFNγ+ CD8+ T cells after PMA/Ionomycin stimulation on day 21. FIG. 15F shows summary bar graphs showing expression levels of CD101, (15g) PD-1, (15h) LAG3, and (15i) CD103 in CD8+ T cells on day 21. FIG. 15J shows summary bar graphs showing the number of viable CD8+ T cells per 100 ul on day 21 for Ch.TCR vs. Ch.TCR treated with BMP4a, or (15k) Ch.TCR treated with TGFβ1 (Dysf.) vs. Dysf. treated with BMP4a. FIG. 15L shows bar graphs showing the expression levels of Ki67 proliferation marker in CD8+ T cells on day 21 for Ch.TCR vs. Ch.TCR treated with BMP4a, or (15m) Dysf. vs. Dysf. treated with BMP4a. N≥4 per group from 2-3 independent experiments. Comparisons were made using the Mann-Whitney U test. * p-value <0.05 as indicated. Error bars indicate mean±SEM.

FIGS. 16A, 16B, 16C, 16D, 16E, 16F, 16G, 16H, 16I, 16J, 16K, 16L, 16M, 16N, 16O, 16P, 16Q, and 16R show boosting BMP signaling while targeting TGFβ1 reprograms terminal T cell dysfunction. FIG. 16A shows a schematic for ex vivo chronic TCR stimulation of human CBMCs for 28 days, in the presence of TGFβ1 and “Reprogramming” treatment regimens (Reprog.I: “weak” chronic TCR+TGFβ1 stimulated cells+RepSox (TGFβR1i) on day 14-28; Reprog.II: same as Reprog.I+BMP4 agonist on day 14-28; and Reprog.III: same as Reprog.II plus vitamin C on day 21-28). FIG. 16B shows overlayed tSNE plot of phenotypic changes within human CD8+ T cells on day 28 for each condition. FIG. 16C shows representative tSNE plots showing protein expression levels of IFNγ, TNFα, CD107a, T-bet, and CD103 in PMA/Ionomycin-stimulated CD8+ T cells on day 28. FIG. 16D shows summary bar graphs showing frequencies of IFNγ-producing, (16e) IFNγ and CD107a co-producing, (16f) IFNγ and TNFα co-producing CD8+ T cells, and expression levels of (16g) IFNγ and (16h) T-bet in IFNγ+ CD8+ T cells after PMA/Ionomycin stimulation on day 28. FIG. 16I shows summary bar graphs showing expression levels of PD-1, (16j) CD101, and (16k) LAG3 in human CD8+ T cells on day 28. FIG. 16L shows summary bar graphs for “strong” TCR stimulated cells showing frequencies of IFNγ and CD107a co-producing CD8+ T cells after PMA/Ionomycin stimulation on day 28, and expression levels of (16m) Perform, (16n) CD101, (16o) CD103, (16p) PD-1, (16q) LAG3, and (16r) IL-7R in CD8+ T cells on day 28. Comparisons were made using the Mann-Whitney U test relative to Dysf. CD8+ T cells or as indicated. * p-value <0.05, ** p<0.01, *** p<0.001, **** p<0.0001. Error bars are the mean SEM.

FIGS. 17A, 17B, 17C, 17D, 17E, 17F, 17G, 17H, and 17I show rebalancing of TGFβ1 and 8MP signaling reverses transcriptional programming of T cell dysfunction. FIG. 17A shows a schematic for RNA-sequencing of human CD8+ T cells sorted on Day 0 (Naïve) and Day 28 following the experimental design as described in FIG. 18a. FIG. 17B shows principal component analysis (PCA) of RNA-seq data for each condition (2 samples each). FIG. 17C shows a scatter plot showing the top significantly enriched datasets (related to exhausted CD8 T cells) by GSEA of the downregulated genes in Dysf. versus Reprogrammed CD8+ T cell populations using the C7 Immune Signature Database (datasets #1-3, 5 are from GSE41867; #4 and 6 are from GSE9650). FIG. 17D shows a Venn diagram of overlapping differentially expressed genes (DEGs) downregulated in Dysf. CD8+ T cells compared to each Reprogrammed condition (Reprog.II, and III). FIG. 17E shows gene ontology enrichment analysis of biological processes linked to the commonly upregulated genes in Reprogrammed versus Dysf. CD8+ T cells. FIG. 17F shows a volcano plot showing differentially expressed genes for Reprog.I versus Reprog.II human CD8+ T cells. FIG. 17G shows a bar graph showing the ChIP-Atlas Enrichment Analysis for transcriptional regulators with enriched binding within 5 Kb+/− TSS of genes that are upregulated in reprogrammed human CD8+ T cells under BMP4 agonist treatment (Reprog.II versus Reprog.I), using embryonic stem cells and pluripotent stem cells databases. FIG. 17H shows over-representation analysis (ORA) of genes upregulated in Dysf. versus Ch.TCR or (17i) Reprogrammed versus Dysf. CD8+ T cells relative to the pan-cancer gene signature of human tumor- infiltrating lymphocytes (TILs).

FIGS. 18A, 18B, 18C, 18D, 18E, 18F, 18G, 18H, and 18I show epigenetic remodeling of dysfunctional human T cells by rebalancing TGFβ11 and 8MP signals. FIG. 18A shows whole-genome bisulfite sequencing of human CD8+ T cells sorted on Day 0 (Naïve), Day 7, or Day 28 (under acute or chronic “weak” TCR stimulation conditions) following the experimental design as described in FIG. 4a. FIG. 18I shows bar graphs showing number of differentially methylated regions (DMRs) in Day 7 CD8+ T cells which were demethylated (white) or methylated (black) compared to naïve cells. FIG. 18C shows bar graph showing numbers of methylated (top) or demethylated (bottom) DMRs in Dysf. CD8+ T cells relative to all other conditions on day 28. FIG. 18D shows a pie chart showing genomic distribution of DMRs in Dysf. CD8+ T cells relative to Reprog.I CD8+ T cells on day 28. FIG. 18E shows pathway enrichment analysis of the demethylated DMRs detected in gene bodies within Reprog.III CD8+ T cells versus Dysf. CD8+ T cells (NCI-Nature 1816 database). FIG. 18F shows targeted methylation analysis of de novo DMR in the TBX21 locus in sorted human CD8+ T cells. FIG. 18G shows methylation percentage plots showing DMRs within the PRF1 and (18h) ITGAE loci for human CD8+ T cells isolated on day 28. Vertical lines indicate CpG positions in the loci. The ratio of blue-to-red indicates the percentage of unmethylated vs. methylated reads, respectively. FIG. 18i shows a Venn diagram of the ChIP-Atlas Enrichment Analysis for transcriptional regulators with binding elements enriched in DMRs that were demethylated during day 7-to-day 28 transition within Dysf. or Chronic TCR stimulated CD8+ T cells. Comparisons in panel f were determined by Mann-Whitney U test. ** p-value <0.01, **** p<0.0001 from at least 2 independent experiments. Error bars are the mean f SEM.

FIGS. 19A, 19B, 19C, 19D, 19E, 19F, 19G, 19H, 19I, 19J, 19K, 19L, 19M, 19N, 19O, and 19P show reprogrammed CD8+ T cells exhibit superior anti-tumor cytotoxic functions. FIG. 19A shows a schematic for co-culture of human CD8+ T cells isolated at Day 7 or Day 28 with a human AML cell line and soluble anti-CD3 for 18 hours. FIG. 19B shows a summary bar graph showing numbers of dead AML cells per 100 viable CD8+ T cells following 18-hour co-culture at day 28, and (19c) fold change in expression levels of CD101 and (19d) LAG3 on viable CD8+ T cells after co-culture, relative to basal expression levels in PMA/Ionomycin stimulated CD8+ T cells on day 28. FIG. 19E shows representative FACS plots of PD-1 and LAG3 expression on viable CD8+ T cells following co-culture. FIG. 19F shows summary bar graph showing numbers of dead MDA-MB-231 cells per 100 viable CD8+ T cells following 18-hour co-culture at day 28. FIG. 19G shows summary bar graphs showing dead MDA-MB-231 cells or (19h) dead AML cells per 100 viable CD8+ T cells from strong TCR-stimulated cells following 18-hour co-culture on day 28. FIG. 19i shows summary of dead AML cells per 100 viable human adult CD8+ T cells under strong TCR following 18-hour co-culture with AML cells on day 28. FIG. 19J shows schematic for co-culture of P14 CD8+ T cells with CT2A-GP33 tumor cells under indicated treatment conditions. FIG. 19K shows summary bar graphs showing dead CT2A-GP33 cells per 100 viable P14 cells, (19l) frequencies of IFNγ and CD107a co-producing and (19m) CD39 and PD-1 co-expressing CD8+ T cells, and (19n) expression level of CD103 on P14 CD8+ T cells on day 16 after PMA/Ionomycin stimulation. FIG. 19O shows experimental setup for tracking B16-F10-LCMV-GP tumor growth during RepSox+/−BMP4 agonist treatment in C57BL/6 mice. FIG. 19P shows longitudinal tracking of tumor volume relative to day 10 or 11 through day 17 for each treatment group. N≥4 per group from 2-3 independent experiments. Comparisons were made using the Mann-Whitney U test (panels b, f-p) or unpaired t test with Welch's correction (panels c-d). * p-value <0.05, ** p<0.01, *** p<0.001 relative to Dysf. CD8+ T cells (b-i), Day 16 vehicle-treated cells (k-n), or as indicated. Error bars are the mean±SEM.

FIGS. 20A, 20B, 20C, 20D, 20E, 20F, 20G, 20H, 20I, 20J, and 20K show therapeutic TGFβR1 blockade plus BMP4 agonist synergizes CD8+ T cell responses to PD-L1 blockade during lifelong chronic LCMV infection. FIG. 20A shows experimental setup for sequential mock or RepSox+/−BMP4 agonist followed by mock or anti-PD-L1 treatment during lifelong chronic LCMV infection in C57BL/6 mice. FIG. 20B shows summary bar graphs showing numbers of P14 (Thy1.1+GP33− tetramer+) CD8+ T cells, (20c) polyclonal LCMV-specific (CD44high PD-1+) CD8+ T cells, (20d) cytolytic (CD44high Cx3cr1+PD-1+) and (20e) progenitor (PD-1+ Tcf1+ Tim-3− Cx3cr1−) CD8+ T cells in spleen following anti-PD-L1 treatment. FIG. 20F shows a summary dot plot of LCMV viral titers (PFU/ml) in serum from chronically LCMV-infected mice after sequential treatments ˜day 48-50 p.i. FIG. 20G shows experimental setup for co-adoptive transfer of ˜100-200K cells of congenically distinct Progenitor (Tim-3− Cx3cr1− PD-1+), Cytolytic (Cx3cr1+PD-1+ Tim3+/−), and Terminally Exhausted (Tim-3+PD-1+Cx3cr1−) LCMV-specific CD8+ T cell subsets into chronically LCMV-infected Rag1 KO-mice, followed by sequential vehicle or RepSox plus BMP4 agonist and anti-PD-L1 treatments. FIG. 20H shows summary plots showing fold change in the numbers of Progenitor and (20l) Terminally Exhausted LCMV-specific CD8+ T cells in the spleen, and (20j) numbers of Cytolytic LCMV-specific CD8+ T cells in the liver and (20k) lungs following anti-PD-L1 treatment. N=4-8 mice per group of two independent experiments. Comparisons were made using the Mann-Whitney U test. * p-value <0.05, ** p<0.01, *** p<0.001. Error bars are the mean±SEM.

FIGS. 21A, 21B, 21C, 21D, 21E, 21F, 21G, 21H, 21I, 21J, 21K, 21L, 21M, 21N, 21O, 21P, 21Q, 21R, 21S, 21T, 21U, and 21V show chronic weak TCR stimulation of human CD8+ T cells does not drive dysfunction. FIG. 21A shows gating strategy showing the phenotype of freshly isolated human CBMCs before ex vivo stimulation. FIG. 21B shows summary bar graphs showing expression levels of IFNγ, (21c) TNFα, (21d) CD107a, (21e) T-bet, and (21f) TCF1 for acutely versus chronically “weak” TCR stimulated IFNγ+ CD8+ T cells after in vitro PMA/Ionomycin stimulation on day 28. FIG. 21G shows longitudinal tracking of CD8+ T cell numbers per 100 ul (left y-axis, solid lines) and frequency of dead CD8 T cells (right y-axis, red dotted lines) under acute or chronic “strong” TCR stimulation from day 0-28. FIG. 21H shows summary bar graphs showing frequency of dead CD8+ T cells on day 28, or expression levels of (21i) CD39, (21j) LAG3, and (21k) CD103 on CD8 T cells under “strong” TCR and TGFβ1 after PMA/Ionomycin stimulation on day 28. FIG. 21L shows schematic of human adult naïve CD8+ T cell isolation followed by acute or chronic stimulation for 28 days. FIG. 21M shows summary bar graphs showing frequency of IFNγ and CD107a-co-producing, (21n) IFNγ and TNFα-co-producing adult CD8+ T cells, (21o) expression levels of CD107a on IFNγ+ CD8+ T cells, and (21p) expression levels of Perform, (21q) CD101, (21r) CD103, (21s) CD39, or (21t) PD-1 on adult CD8+ T cells after PMA/Ionomycin stimulation on day 28. FIG. 21U shows expression levels of CD103 and 21v) frequencies of IFNγ+ TNFα+ CD8+ T cells among adult polyclonal effector memory CD8+ T cells (TEM) after PMA/Ionomycin stimulation on day 28 at the indicated conditions. N≥4 per group from 2-3 independent experiments. Comparisons were made using the Mann-Whitney U test (panels b-k), or unpaired t-test with Welch's correction (panels m-v). * p-value <0.05, ** p<0.01, *** p<0.001. Error bars are the mean±SEM.

FIGS. 22A, 22B, 22C, 22D, 22E, 22F, 22G, and 22H chronic TGFβ1 and TCR signals establish a stable dysfunctional program in CDS+ T cells. FIG. 22A shows summary bar graphs showing expression levels of IFNγ, (22b) T-bet, (22c) TNFα, and (22d) CD107a in IFNγ + CD8+ T cells after 7 days (Day 35) of resting from chronic or acute stimulation as show in FIG. 14a. FIG. 22E shows summary bar graphs showing expression levels of TNFα, and (22f) CD107a in IFNγ+ CD8+ T cells after 14 days of rest (Day 42). FIG. 22G shows summary bar graph showing expression levels of GZMB, and (22h) CD11a in CD8+ T cells after 14 days of rest (Day 42). N≥3 per group from 2-3 independent experiments. Comparisons were made using the unpaired t test with Welch's correction. * p-value <0.05, ** p<0.01, *** p<0.001, **** p<0.0001 relative to Dysf. condition. Error bars are the mean±SEM.

FIGS. 23A, 23B, 23C, 23D, 23E, 23F, 23G, 23H, 23I, and 23J show augmenting BMP signaling while blocking TGFβ1 recovers effector function in dysfunctional CD8+ T cells. FIG. 23A shows IGV Snapshots showing: DNA methylation levels (top panel) at individual CpG sites within mouse naïve (black), antigen- specific exhausted WT (red) or Dnmt3a-deficient (green) CD8+ T cells on day 35 post-chronic LCMV infection. Blue-to-red ratio indicates % of unmethylated versus methylated reads (GSE99450); open chromatin peaks (middle panel) detected in naïve (black), progenitor (PD-1+ Tim-3−; green), or terminally exhausted (PD-1+ Tim-3+; red) CD8+ T cells from chronically infected mice (PRJNA546023); and levels of H3K27 acetylation marks (bottom panel) in the progenitor (green), cytolytic (blue; Cx3cr1+PD-1+), and terminally exhausted (Cx3cr1− PD-1+ Tim-3+) CD8 T cells during chronic LCMV infection (GSE149810) at the Smad1, Smad5, and Tgfbr3 loci. FIG. 23B shows a heatmap of RNA levels in exhausted T cell subsets during chronic LCMV infection (GSE84105). FIG. 23C shows tSNE plot visualization of human CD8+ T cells for the described conditions in FIG. 4a after PMA/ionomycin stimulation on day 28. FIG. 23D shows representative FACS plots showing the gating strategy for sorting dysfunctional human CD8+ T cells on day 14 after chronic TCR plus TGFβ1 stimulation, and day 28 expression of IFNγ and CD103 in PMA/ionomycin-stimulated CD8+ T cells after treatment from day 14-28. FIG. 23E shows summary bar graphs showing frequency of IFNγ and TNFα co-producing CD8+ T cells, and (23f) expression level of CD101 in sorted CD8+ T cells after PMA/ionomycin stimulation on day FIG. 23G shows summary bar graphs showing expression levels of TOX in acutely or chronically stimulated cord blood-derived CD8+ T cells. FIG. 23H shows bar graphs showing expression levels of TOX in adult blood-derived CD8+ T cells on day 21 of chronic “strong” or “weak” TCR stimulation. FIG. 23I shows summary bar graphs showing expression levels of IL-7R and (23j) CD103 in adult CD8+ T cells on day 21 of “weak” TCR stimulation. N≥3 per group from 2-3 independent experiments. Comparisons were made using the unpaired t test with Welch's correction (panels e-f) or Mann-Whitney U test (panels g-j) relative to Dysf. T cells or as indicated * p-value <0.05, ** p<0.01, *** p<0.001, **** p<0.0001. Error bars are the mean SEM.

FIGS. 24A, 24B, 24C, 24D, 24E, 24F, and 24G show transcriptional reprogramming of dysfunctional human CD8+ T cells. FIG. 24A shows volcano plot showing differentially expressed genes in acutely TCR stimulated versus naïve human CD8+ T cells, or (24c) in chronically TCR stimulated versus Dysfunctional human CD8+ T cells. FIG. 24B shows gene set enrichment analysis for Ac.TCR versus Naïve CD8+ T cells or (24d) Ac.TCR versus Dysf. CD8+ T cells compared against common upregulated genes in human CD8+ T cell subsets following yellow fever vaccination⁴⁴. FIG. 24E shows heatmap of differentially expressed genes (DEGs) between each condition based on relative Z-score and organized into clusters: C1 (yellow; up in Naïve and Dysf.), C2 (orange; up in Dysf. only), C3 (blue: up in Ch.TCR and Ac.TCR), C4 (green; up in Reprog.I-III), C5 (purple; up in Ch.TCR, Ac.TCR, and Reprog.I-III), and C6 (pink; up in Naïve, Ch.TCR, Ac.TCR, and Reprog.I-III). Example genes with significant fold change in expression (fold change >2, p<0.05) are listed per cluster. FIG. 24F shows a Venn diagram of overlapping genes upregulated in Dysf. versus Reprog.I-III CD8+ T cells. FIG. 24G shows gene set enrichment analysis of biological pathways linked to the upregulated genes in Dysf. versus Reprog.I-III CD8+ T cells using Hallmark gene signature database.

FIGS. 25A, 25B, 25C, 25D, 25E, 25F, 25G, 25H, 25I, 25J, 25K, 25L, 25M, and 25N show BMP4 agonist treatment promotes transcriptional recovery of dysfunctional CD8+ T cells. FIG. 25A shows a Venn diagram of overlapping genes that are downregulated in Dysf. versus Reprog.I-III CD8+ T cells on day 28. FIG. 25B shows pathway enrichment analysis of genes that are upregulated in Reprog.II versus Dysf. CD8+ T cells or overlap with the upregulated genes in Reprog.III versus Dysf. CD8+ T cells (NCI-Nature 2016 pathway database). FIG. 25C shows interaction plots from RNA-seq data showing average expression levels of KLF3, (25d) SIPR1, (25e) GZMK, (25f) FCGR3A, (25g) PRF1, (25h) CX3CR1, (25i) TGPBR3, (25j) CD28, (25k) CD109, (25l) MYO7A, (25m) ITGAE, and (25n) SMAD6 transcripts among chronically (Ch.TCR, Dysf., Reprog.I-III) or acutely (Ac.TCR) stimulated CD8+ T cells on day 28.

FIGS. 26A, 26B, and 26C show chronic TGFβ1 exposure induces similar transcriptional features of terminally exhausted CDS+ T cells in human cancers. FIG. 26A shows UMAP visualization of CD8+ T cell meta-clusters identified in single-cell RNA-seq analysis of human CD8+ T cell subsets including tumor-infiltrating lymphocytes (TILs) from 21 types of human cancer. FIGS. 26B and 26C show UMAP visualization showing RNA expression levels of some selected signature genes in human TIL and memory CD8+ T cell clusters that overlap with transcriptional features of in vitro-differentiated dysfunctional and reprogrammed human CD8+ T cells.

FIGS. 27A, 27B, 27C, 27D, 27E, 27F, 27G, 27H, and 27I show reprogrammed CD8+ T cells maintain a heritable functional memory-like program. FIG. 27A shows representative FACS plots showing CD8+ T cell divisions and expression levels of IL-7R in CFSE-labelled CD8+ T cells from each condition as described in FIG. 18a on day 35 after 7 day-rest under homeostatic conditions. FIG. 27B shows expression levels of IL-7R in divided CD8+ T cells on day 35. FIG. 27C shows representative FACS plots showing CD8+ T cell divisions and expression levels of CD103 in CFSE-labelled CD8+ T cells on day 35. FIG. 27D shows expression levels of CD103 in divided CD8+ T cells on day 35 (27e) Summary bar graphs for frequencies of divided CD8+ T cells (≥3 proliferation cycles), and (27f) expression levels of IL-7R and (27g) CD103 in CD8+ T cells on day 35. FIG. 27H shows summary bar graphs showing the expression levels of TNFα and (27i) Perform in IFNγ+ CD8+ T cells after PMA/Ionomycin stimulation on day 35. N≥4 per group from 2-3 independent experiments. Comparisons were made using Mann-Whitney U test (panel e-i) or unpaired t test with Welch's correction (panel b, d) relative to Dysf. condition or as indicated. * p-value <0.05, ** p<0.01, *** p<0.001, **** p<0.0001. Error bars are the mean±SEM.

FIGS. 28A, 28B, 28C, and 28D show BMP signals promote reprogramming of CD8+ T cells away from terminal dysfunction. FIG. 28A shows Venn diagrams showing transcription factor (TF)-binding motifs enriched in hypermethylated or (28b) hypomethylated genomic regions in in Dvsf. versus Reprog.I, IL or III human CD8+ T cells on day 28. FIG. 28C shows snapshots from scATAC-seq database showing chromatin accessibility changes at selected loci of effector and memory, or (28d) dysfunctional programs in human CD8+ T cell subsets from patients with cancer.

FIGS. 29A, 29B, 29C, 29D, 29E, 29F, 29G, 29H, 29I, 29J, and 29K show reprogramming of dysfunctional CD8+ T cells improves cytotoxicity against AML and immunotherapy-resistant metastatic breast adenocarcinoma. FIG. 29A shows representative FACS plots showing E-cadherin and PD-L1 expression on THP-1 (AML) and MDA-MB-229 (breast adenocarcinoma) tumor cells. FIG. 29B shows summary bar graphs showing frequencies of PD-1+ LAG3+, (29c) total LAG3+ CD8+ T cells, or (29d) expression levels of PD-1 and (29e) GZMB on CD8+ T cells following 18-hour co-culture with AML cells at day 28. FIG. 29F shows summary bar graph showing expression levels of CD103 on CD8+ T cells following co-culture with MDA-MB-229 cells at day 28. FIG. 29G shows summary bar graph showing frequency of LAG3 and CD101 co-expressing CD8+ T cells under “strong” TCR stimulation following 18-hour co-culture with AML cells at day 28. FIG. 29H shows summary of dead AML cells per 100 viable CD8+ T cells under “strong” TCR following 18-hour co-culture with AML cells at varying effector: target ratios on day 28. FIG. 29I shows summary bar graph showing dead AML cells per 100 viable CD8+ T cells from isolated adult naïve or effector memory (TEM) CD8+ T cells under “weak” TCR following 18-hour co-culture at day 28. FIG. 29J shows summary bar graph of dead AML cells per 100 viable CD8+ T cells after rest from “strong” TCR under homeostatic conditions from day 28 to 35, following 18-hour co-culture with AML cells on day 35. FIG. 29K shows expression levels of LAG3 in CD8+ T cells after PMA/lonomycin stimulation on day 35 following rest from “strong” TCR for 7 days. N≥4 per group from 2-3 independent experiments. Comparisons were made using Mann-Whitney U test relative to Dysf. condition or as indicated. * p-value <0.05, ** p<0.01. Error bars are the mean±SEM.

FIGS. 30A, 30B, 30C, 30D, 30E, 30F, and 30G show combined TGFβR1 blockade and BMP4 agonist treatment enhances rejuvenation of CD8+ T cells following ICB therapy. FIG. 30A shows summary bar graphs showing numbers of polyclonal LCMV-specific (CD44hi PD-1+), (30b) proliferating (Ki67+) CD44hi PD-1+, (30c) Cytolytic (CD44high Cx3cr1+PD-1+) and (30d) Terminally Exhausted (Tim3+PD-1+Cx3cr1−) CD8+ T cells in spleen on day 33 following primary treatment using RepSox, BMP4a, or combined treatment as described in FIG. 8a. FIG. 30E shows summary dot plot of LCMV viral titers (PFU/ml) in serum from chronically LCMV-infected mice on day 33 following primary treatment. FIG. 30F shows summary bar graphs showing numbers of proliferating (Ki67+ CD44high PD-1+) CD8+ T cells, and (30g) proliferating P14 (Ki67+ Thy1.1+GP33-tetramer+) CD8+ T cells in spleen following anti-PD-L1 treatment. N≥4 per group from 2-3 independent experiments. Comparisons were made using Mann-Whitney U test. * p-value <0.05. ** p<0.01. *** p<0.001. Error bars are the mean±SEM.

FIG. 31 shows rebalancing TGFβ1/BMP-signals in exhausted T cells unlocks responsiveness to ICB therapy.

FIG. 32 shows levels of BMP receptors in human CD8 T cells under chronic stimulation. Normalized RNA levels in human CD8 T cells during chronic stimulation (GSE217072-PMID: 36543960)

FIG. 33 shows treatment of chronically stimulated human CD8 T cells with BMP.

FIGS. 34A and 34B shows that BMP4 ligand treatment maintains survival and effector functions of chronically stimulated human CD8 T cells. FIG. 34A shows bar graph showing human CD8 T cell numbers after treatment with BMP ligands under chronic strong TCR stimulation. FIG. 34B shows line plot showing the frequencies of IFNγ-producing human CD8 T cells on days 14 and 21 during chronic stimulation. * P-value<0.05, ** <0.01 using one-way ANOVA (A) or two-way ANOVA test (B)

FIGS. 35A, 35B, and 35C show that BMP2 ligand treatment increases expression of terminal exhaustion-related molecules on human CD8 T cells. Bar graphs showing surface expression levels of (35A) CD39, (35B) CD101, and (35C) CD103 proteins on human CD8 T cells on day 21 after treatment with BMP ligands under chronic strong TCR stimulation. *** P-value<0.001, **** <0.0001 using one-way ANOVA test.

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327: 167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus and includes adaptation molecules as well as interference molecules. An interference molecule of a bacterial adaptive immunity complex includes endonucleases. A Cas endonuclease described herein comprises one or more nuclease domains. Contemplated herein are any Cas molecules, including Type 1, Type II, and Type II.

TABLE A
Cas Subtypes.

| Cas subtype | Signature protein |
|---|---|
| — | Cas3 |
| I-A | Cas8a, Cas5 |
| I-B | Cas8b |
| I-C | Cas8c |
| I-D | Cas10d |
| I-E | Cse1, Cse2 |
| I-F | Csy1, Csy2, Csy3 |
| I-G | GSU0054 |
| — | Cas10 |
| III-A | Csm2 |
| III-B | Cmr5 |
| III-C | Cas10 or Csx11 |
| III-D | Csx10 |
| III-E | |
| III-F | |
| — | Csf1 |
| IV-A | |
| IV-B | |
| IV-C | |
| — | Cas9 |
| II-A | Csn2 |
| II-B | Cas4 |
| II-C | |
| — | Cas12 |
| V-A | Cas12a (Cpf1) |
| V-B | Cas12b (C2c1) |
| V-C | Cas12c (C2c3) |
| V-D | Cas12d (CasY) |
| V-E | Cas12e (CasX) |
| V-F | Cas12f (Cas14, C2c10) |
| V-G | Cas12g |
| V-H | Cas12h |
| V-I | Cas12i |
| V-K^{[Note 2]} | Cas12k (C2c5) |
| V-U | C2c4, C2c8, C2c9 |
| — | Cas13 |
| VI-A | Cas13a (C2c2) |
| VI-B | Cas13b |
| VI-C | Cas13c |
| VI-D | Cas13d |
| VI-X | Cas13x.1 |
| VI-Y | |

As used herein, the term “Cas9 protein” refers to, but is not limited to, Cas9 proteins, Cas9-type proteins encoded by Cas9 orthologs, and synthetic proteins of Cas9. The term “Cas9 protein” as used herein refers to a wild type Cas9 protein from CRISPR-Cas9 type II B systems, Cas9 protein modifications, Cas9 protein variants, Cas9 orthologs and combinations of the same. The term “dCas9” as used herein refers to Cas9 protein variants that are Cas9 proteins deactivated by nuclease, also referred to as “catalytically inactive Cas9 protein”, or “enzymatically inactive Cas9”. Various Cas9s and their relationship with each other can be found in Gasiunas, et al. (Gasiunas G., Young, J. K., Karvelis, T. et al. A catalogue of biochemically diverse CRISPR-Cas9 orthologs. Nat Commun 11, 5512 2020, hereby incorporated by reference in its entirety for its discussion concerning Cas9 molecules).

A Cas protein is further defined as a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “target site”, “target sequence”, “target site sequence,” target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature.

A “protospacer adjacent motif” (PAM) herein refers to a short nucleotide sequence adjacent to a target sequence (protospacer) that is recognized (targeted) by a guide polynucleotide/Cas endonuclease system described herein or a non-target sequence that is complementary to the target sequence. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular TGFβR1 inhibitor or BMP4 agonist is disclosed and discussed and a number of modifications that can be made to a number of molecules including the TGFβR1 inhibitor or BMP4 agonist are discussed, specifically contemplated is each and every combination and permutation of TGFβR1 inhibitor or BMP4 agonist and the modifications that are possible unless specifically indicated to the contrary. Thus, if a class of molecules A, B, and C are disclosed as well as a class of molecules D, E. and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Cytotoxic CD8+ T cell function and the generation of antigen-specific memory cells are critical for adaptive immunity against viral infections and cancer. However, during chronic stimulation, both effector functions and the memory potential of CD8+ T cells are gradually lost (partial “exhaustion”), culminating in terminal dysfunction, which often results in failure to clear pathogens or tumors. By targeting inhibitory molecules such as PD-1 and CTLA-4, immune checkpoint blockade (ICB) can reinvigorate partially exhausted T cells, an approach that has shown therapeutic success for treating multiple types of cancer. Despite these advances, many patients remain nonresponsive to ICB or relapse after developing an initial clinical response. Poor treatment outcomes have been linked to an ICB-irreversible, terminally dysfunctional state within tumor-infiltrating CD8+ T cells. Therefore, extensive efforts have focused on defining the molecular determinants of T cell dysfunction that could be blocked or reversed to improve ICB efficacy.

In this regard, CD8+ T cell dysfunction occurs progressively during chronic stimulation, transiting through three major subsets: a) early dysfunctional or “progenitor” cells that retain a degree of proliferative potential and functional capacity; b) a highly cytolytic subset; and c) fully exhausted or terminally dysfunctional T cells that are characterized by loss of effector cytokine secretion (e.g., IFNγ, TNFα, IL-2), impaired homeostatic proliferation, and mitochondrial dysfunction. The progenitor and cytolytic subsets are indispensable for responding to ICB therapy, while terminally dysfunctional cells are refractory and represent the major barrier to the efficacy of T cell-directed immunotherapies.

A key component of the progression toward terminal dysfunction is epigenetic reprogramming, including changes in DNA methylation and histone modification landscapes, which regulate gene expression patterns, driving T cells toward either functional memory or dysfunctional fates. Indeed, epigenome analyses revealed that terminally dysfunctional CD8+ T cells acquire distinct open chromatin and DNA methylation landscapes relative to those within effector and memory subsets. Importantly, de novo DNA methylation, mediated by the Dnmt3a enzyme, plays a fundamental role in driving terminal dysfunction in chronically stimulated CD8+ T cells, further limiting their response to ICB therapy.

While ICB treatment transiently expands and restores effector functions to partially exhausted T cells at the transcriptional level, limited changes occur in chromatin accessibility at genes encoding key effector molecules and inhibitory receptors. As a result, rejuvenated CD8+ T cells eventually progress towards terminal dysfunction, potentially explaining the lack of durable T cell responses in many patients. Likewise, after the resolution of chronic infections, virus-specific CD8+ T cells exhibit a limited functional recovery. The acquisition of stable epigenetic programs that enforce terminal dysfunction remain as T cell-intrinsic “scars”, blocking the recovery of effector/memory potential. Paradoxically, compared with their virus- or tumor-specific counterparts, self-reactive T cells maintain effector functions and sternness features despite prolonged exposure to auto-antigens. Such disparate outcomes of chronic antigen exposure suggest that microenvironmental cues are important in modulating T cell fate decision. Hence, identification of key signals that regulate epigenetic programming in dysfunctional CD8+ T cells would reveal new targets to boost T cell immunotherapies.

To determine which microenvironmental signals impart epigenetic programs for terminal dysfunction upon human CD8+ T cells, and whether these programs can be reversed, we developed a novel in vitro model system. We discovered that post-effector stage, chronic TGFβ1 exposure, coupled with persistent T cell receptor (TCR) stimulation, drives terminal CD8+ T cell dysfunction, as well as epigenetic changes associated with impaired effector function and memory potential. Simultaneous impairment of TGFβ1 and potentiation of BMP signaling restored effector function and memory programs via re-wiring transcriptional circuits in terminally dysfunctional CD8+ T cells. Importantly, this rebalancing of TGFβ1/BMP signals enhanced the cytotoxic capacity of human dysfunctional CD8+ T cells when challenged with tumors, enhanced control of established tumors in mice, and boosted dysfunctional CD8+ T cell responses to ICB therapy and viral control during lifelong chronic lymphocytic choriomeningitis (LCMV) infection. Our findings indicate that relative levels of TGFβ3 family members in a tumor microenvironment are critical determinants of functional capacity for chronically stimulated CD8+ T cells, revealing a potential new strategy to epigenetically reprogram terminally dysfunctional T cells during ICB therapy.

In one aspect, disclosed herein are cancer or infectious disease treatment regimens comprising 1) one or more checkpoint inhibitors, 2) one or more transforming growth factor-β receptor 1 (TGFβR1) inhibitors, and 3) one or more bone morphogenic protein 4 (BMP4), BMP6, or BMP10 protein, and/or a BMP4, BMP6, or BMP10 agonists.

“Immune checkpoint inhibitors” are well known in the art. As used herein, “immune checkpoint inhibitors” refers to any small molecule or antibody that blocks checkpoint receptors interacting with their ligand or a ligand from binding to its receptor, or interferes with signaling of a checkpoint receptor. Examples of checkpoint inhibitors include, but are not limited to antibodies that block PD-1 (such as, for example, Nivolumab (BMS-936558 or MDXI 106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS1T8, MGD013, and Immutep).

TGFbR1 inhibitors can include genetic modification of TGFbR1 (such as, for example targeted knockout of the gene) or the use of antibodies or small molecules that bind to TGFbR1 and prevent signaling. Examples of such small molecules, include, but are not limited to RepSox, (also known as E-616452) (2-(3-(6-Methylpyridine-2-yl)-1H-pyrazol-4-yl)-1,5-naphthyridine) as shown in the formula:

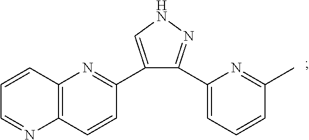 [SMILES: Cc1cccc(-c2n[nH]cc2-c2ccc3ncccc3n2)n1]

SB525334 (6-[2-tert-Butyl-5-(6-methyl-pyridin-2-yl)-1H-imidazol-4-yl]-quinoxaline) as shown in the formula:

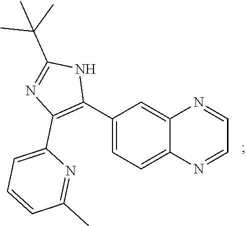 [SMILES: Cc1cccc(-c2nc(C(C)(C)C)[nH]c2-c2ccc3nccnc3c2)n1]

GW788388 (4-(4-(3-(pyridin-2-yl)-1H-pyrazol-4-yl)pyridin-2-yl)-N-(tetrahydro-2H-pyran-4-yl)benzamide) as shown in the formula:

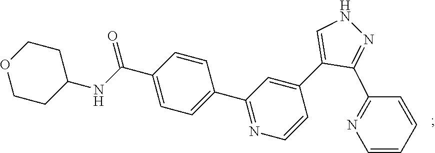 [SMILES: O=C(NC1CCOCC1)c1ccc(-c2cc(-c3c[nH]nc3-c3ccccn3)ccn2)cc1]

Vactosertib (TEW-7197) (2-fluoro-N-[[5-(6-methylpyridin-2-yl)-4-([1,2,4]triazolo[1,5-a]pyridin-6-yl)-1H-imidazol-2-yl]methyl]aniline) as shown in the formula:

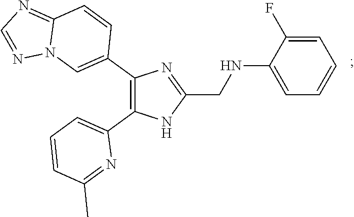 [SMILES: Cc1cccc(-c2[nH]c(CNc3ccccc3F)nc2-c2ccc3ncnn3c2)n1]

SD-208 (2-(5-Chloro-2-fluorophenyl)-N-4-pyridinyl-4-pteridinamine) as shown in the formula:

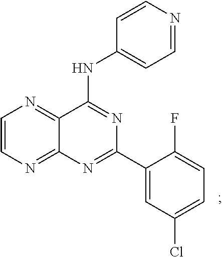 [SMILES: Fc1ccc(Cl)cc1-c1nc(Nc2ccncc2)c2nccnc2n1]

Galunisertib (LY2157299) (4-[2-(6-methylpyridin-2-yl)-5,6-dihydro-4H-pyrrolo[1,2-b]pyrazol-3-yl]quinoline-6-carboxamide) as shown in the formula:

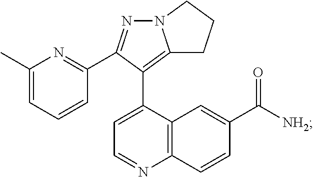 [SMILES: Cc1cccc(-c2nn3c(c2-c2ccnc4ccc(C(N)=O)cc24)CCC3)n1]

and
LY3200882 (4-[[4-[[1-cyclopropyl-3-(tetrahydro-2H-pyran-4-yl)-1H-pyrazol-4-yl]oxy]-2-pyridinyl]amino]-α,α-dimethyl-2-pyridinemethanol) as shown in the formula:

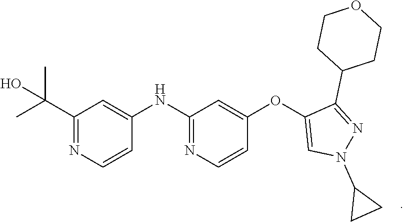 [SMILES: CC(C)(O)c1cc(Nc2cc(Oc3cn(C4CC4)nc3C3CCOCC3)ccn2)ccn1]

90. Alternatively, targeted deletion or disruption of the TGFβR1 gene can be used to inhibit TGFβR1. For example, vector encoding a CRISPR/Cas9 endonuclease integration system comprising a guide RNA (gRNA) that targets TGFβR1 gene can be used to disrupt all or a portion of the TGFβR1 gene.

Bone morphogenic protein 4 is a protein belonging to the TGFβ superfamily of proteins. As shown herein potentiation of BMP signaling restored effector function and memory programs in exhausted T cells. As shown herein, the treatment regimens comprising BMP4 or a BMP4 agonist can help restore T cell functionality in a previously exhausted T cell. Examples of BMP agonists include, but are not limited to SB4 (2-[[(4-Bromophenyl)methyl]thio]benzoxazole), as shown in the formula:

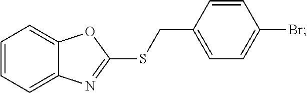 [SMILES: Brc1ccc(CSc2nc3ccccc3o2)cc1]

SJ000291942 (2-(4-Ethylphenoxy)-N-(4-fluoro-3-nitrophenyl)acetamide) as shown in the formula:

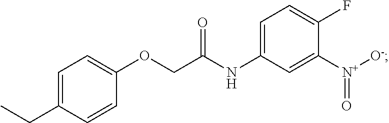

SJ000063181 as shown in the formula:

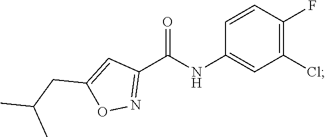 [SMILES: CC(C)Cc1cc(C(=O)Nc2ccc(F)c(Cl)c2)no1]

and
SJ00037178 as shown in the formula

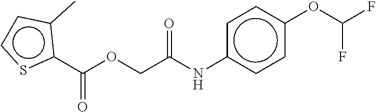

In one aspect, the regimen further comprises an antioxidant, including, but not limited to vitamin C.

The components of the treatment regimen (i.e., the immune checkpoint inhibitor, the TGFβR1 inhibitor, and the BMP4 or BMP4 agonist) can be administered as a single composition or as multiple compositions. Where administered separately, the components can be administered concurrently or at any combination of different times. For example, the TGFβR1 inhibitor and the BMP4 or BMP4 agonist can be administered as a single composition or concurrently and prior to or after administration of the checkpoint inhibitor. Alternatively, the TGFβR1 inhibitor and the checkpoint inhibitor can be administered as a single composition or concurrently and prior to or after administration of the BMP4 or BMP4 agonist. In another aspect, the BMP4 or BMP4 agonist and the checkpoint inhibitor can be administered as a single composition or concurrently and prior to or after administration of the TGFβR1 inhibitor. In one aspect, the TGFβR1 inhibitor and the BMP4 or BMP4 agonist can be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 40, 45, 50, 55, 58, 59, 60, 61, 75, or 90 days 4, 5, 6, 7, 8, 9, 10, 11, or 12 months prior to or following administration of the checkpoint inhibitor.

### 1. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne. T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes ß-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR- cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 2. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with TGFβR1 such that TGFβR1 is inhibited from signaling. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques. e.g., as described in U.S. Pat. No. 5,804,440 to Burton et al, and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, scFv, and the like, including hybrid fragments. Thus, fragments of the antibodies that retain the ability to bind their specific antigens are provided. For example, fragments of antibodies which maintain TGFβR1 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature. 321:522-525 (1986). Riechmann et al., Nature, 332:323-327 (1988). Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti TGFβR1 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 3. Antioxidants

In one aspect, the methods described herein can further comprise the administration of an antioxidant, including, but not limited to Vitamin C. Generally, antioxidants are compounds that get react with, and typically get consumed by, oxygen. Since antioxidants typically react with oxygen, antioxidants also typically react with the free radical generators, and free radicals. (“The Antioxidants—The Nutrients that Guard Your Body” by Richard A. Passwater, Ph. D., 1985, Keats Publishing Inc., which is herein incorporated by reference at least for material related to antioxidants). The compositions can contain any antioxidants, and a non-limiting list would included but not be limited to, non-flavonoid antioxidants and nutrients that can directly scavenge free radicals including multi-carotenes, beta-carotenes, alpha-carotenes, gamma-carotenes, lycopene, lutein and zeanthins, selenium. Vitamin E, including alpha-, beta- and gamma- (tocopherol, particularly .alpha.-tocopherol, etc., vitamin E succinate, and trolox (a soluble Vitamin E analog) Vitamin C (ascoribic acid) and Niacin (Vitamin B3, nicotinic acid and nicotinamide), Vitamin A, 13-cis retinoic acid, N-acetyl-L-cysteine (NAC), sodium ascorbate, pyrrolidin-edithio-carbamate, and coenzyme Q10; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates O₂H₂O₂; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavenoids and flavenoid like molecules like folic acid and folate. A review of antioxidant enzymes and mimetics thereof and antioxidant nutrients can be found in Kumar et al, Pharmac. Ther. Vol 39: 301, 1988 and Machlin L. J. and Bendich, F.A.S.E.B. Journal Vol. 1:441-445, 1987 which are incorporated herein by reference for material related to antioxidants.

Flavonoids, also known as “phenylchromones.” are naturally occurring, water-soluble compounds which have antioxidant characteristics. Flavonoids are widely distributed in vascular plants and are found in numerous vegetables, fruits and beverages such as tea and wine (particularly red wine). Flavonoids are conjugated aromatic compounds. The most widely occurring flavonoids are flavones and flavonols (for example, myricetin, (3,5,7,3′,4′,5′,-hexahydroxyflavone), quercetin (3,5,7,3′,4′-pentahydroxyflavone), kaempferol (3,5,7,4′-tetrahydroxyflavone), and flavones apigenin (5,7,4′-trihydroxyflavone) and luteolin (5,7,3′,4′-tetrahydroxyflavone) and glycosides thereof and quercetin).

### 4. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe. K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press. New York (1977) pp. 365-389. A typical daily dosage of the antibody used alone might range from about 1 μg/kg to up to 100 mg/kg of body weight or more per day, depending on the factors mentioned above.

### C. METHODS OF USING THE COMPOSITIONS

Cytotoxic CD8 T lymphocytes are an important defense against tumors or virus-infected cells; progressively lose their killing function and become exhausted during cancer or chronic virus infections. Exhaustion remains a major challenge to T cell immunotherapy. In some cases, this barrier can be surmounted with immune checkpoint blockade (ICB), which rejuvenates partially-exhausted T cells by blocking signals from inhibitory receptors (e.g., CTLA-4, PD-1/PD-L1). Despite the success of ICB in treating some previously refractory cancers, many patients remain nonresponsive. As such, it remains a crucial goal to identify and target molecular barriers for rejuvenation of fully-exhausted T cells that remain refractory to ICB therapy. Herein we show how full exhaustion is acquired in T cells, and how to reprogram fully-exhausted T cells, rescuing their responsiveness to ICB therapy.

In one aspect, disclosed herein are methods of rescuing the functional phenotype of exhausted T cells in a subject in need thereof comprising administering to the subject a 1) transforming growth factor-β receptor 1 (TGFβR1) inhibitor (such as, for example, RepSox. SB525334, GW788388, Vactosertib, SD-208, Galunisertib, and/or LY3200882 or a vector encoding a CRISPR/Cas9 endonuclease integration system comprising a guide RNA (gRNA) that targets TGFβR1 gene) and 2) a bone morphogenic protein 4 (BMP4), BMP6, BMP10 protein, or a BMP4, BMP6, or BMP10 agonist (such as, for example, the BMP4 agonists SB4, SJ000063181, SJ000291942, and/or SJ000370178). In one aspect, the method further comprises the administration of an antioxidant, including, but not limited to vitamin C.

In one aspect, disclosed herein are methods of rescuing the functional phenotype of exhausted T cells in a subject in need thereof comprising administering to the subject a vector (such as, for example, an adeno-associated virus (AAV) vector including, but not limited to AAV1, AAV2, AAV3, AAV4, AAV5, AAV6, AAV7, AAV8, or AAV9) encoding clustered regularly interspaced short palindromic repeat (CRISPR)/CRISPR-associated 9 (Cas9) endonuclease integration system wherein the Cas9 endonuclease complexed with a guide RNA (gRNA) that targets TGFβR1 gene; and wherein expression of the CRISPR/Cas9 endonuclease integration systems excises all or a functional fragment of the TGFβR1. In one aspect, the expression of the Cas9 endonuclease is operatively linked to a T cell specific promoter, inducible promoter, or constitutive promoter).

Also disclosed herein are methods of increasing the efficacy of or reducing resistance to an immune checkpoint blockade in a subject receiving treatment with an immune checkpoint inhibitor comprising administering to the subject 1) one or more transforming growth factor-β receptor 1 (TGFβR1) inhibitors (such as, for example, RepSox, SB525334, GW788388, Vactosertib, SD-208. Galunisertib. and/or LY3200882 or a vector encoding a CRISPR/Cas9 endonuclease integration system comprising a guide RNA (gRNA) that targets TGFβR1 gene) and 2) one or more bone morphogenic protein 4 (BMP4), BMP6, or BMP10 protein, and/or a BMP4, BMP6, or BMP10 agonists (such as, for example, the BMP4 agonists SB4, SJ000063181, SJ000291942, and/or SJ000370178).

In one aspect, disclosed herein are methods of increasing the efficacy of or reducing resistance to an immune checkpoint blockade in a subject, wherein the immune checkpoint inhibitor includes, but is not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, T cell immunoreceptor with Ig and ITIM domains (TIGIT)(such as, for example BMS-986207. OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, B1754111, Sym022, FS118, MGD013, and Immutep).

To rescue the efficacy of an immune checkpoint blockade or or reducing resistance to an immune checkpoint blockade, it is understood and herein contemplated that the TGFβR1 inhibitor and the BMP4 or BMP4 agonist can be administered as a single composition or as multiple compositions. This administration (either with the TGFβR1 inhibitor and the BMP4 or BMP4 agonist as a single formulation or separately) can again be formulated separately or in combination with the checkpoint inhibitor. Where administered separately, the components can be administered concurrently or at any combination of different times. For example, the TGFβR1 inhibitor and the BMP4 or BMP4 agonist can be administered as a single composition or concurrently and prior to or after administration of the checkpoint inhibitor. Alternatively, the TGFβR1 inhibitor and the checkpoint inhibitor can be administered as a single composition or concurrently and prior to or after administration of the BMP4 or BMP4 agonist. In another aspect, the BMP4 or BMP4 agonist and the checkpoint inhibitor can be administered as a single composition or concurrently and prior to or after administration of the TGFβR1 inhibitor. In one aspect, the TGFβR1 inhibitor and the BMP4 or BMP4 agonist can be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 40, 45, 50, 55, 58, 59, 60, 61, 75, or 90 days 4, 5, 6, 7, 8, 9, 10, 11, or 12 months prior to or following administration of the checkpoint inhibitor. Thus, in some aspects, the TGFβR1 inhibitor and the BMP4 or BMP4 agonist are administered before immune checkpoint blockade begins or after IBC has been used and shown to have lost its efficacy.

Administration each component can occur a single time or multiple times for any duration as determined necessary by the practicing physician. For example, the the TGFβR1 inhibitor and the BMP4 or BMP4 agonist can be administered 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times with administrations occurring once every 1, 2, 3, 4, 5, 6, 7, 8, 9, 10, 11, 12, 13, 14, 15, 16, 17, 18, 19, 20, 21, 22, 23, 24, 30, 36, 42, 48 hours, 3, 4, 5, 6, 7, 8, 9, 10, 11, 12, 13, 14, 15, 16, 17, 18, 19, 20.21, 22, 23, 24, 25, 26, 27, 28, 29, 30 days, 2, 3, 4, 5, 6, 7, 8, 9, 10, 11, or 12 months.

### 1. Methods of Treating Infectious Disease

It is understood and herein contemplated that the disclosed compositions can be used to treat any disease caused by an infectious agent or mitigate the symptoms thereof. Accordingly, in one aspect, disclosed herein are methods of treating, inhibiting, reducing, decreasing, ameliorating, and/or preventing an infectious disease (such as, for example an acute or chronic viral infection, bacterial infection, parasitic infection, or fungal infection) in a subject, the method comprising administering to the subject any of the treatment regimens disclosed herein. For example, disclosed herein are methods of treating, inhibiting, reducing, decreasing, ameliorating, and/or preventing a cancer and/or metastasis in a subject or methods of treating, inhibiting, reducing, decreasing, ameliorating, and/or preventing an infectious disease (such as, for example a viral infection, bacterial infection, parasitic infection, or fungal infection) in a subject, the method comprising administering to the subject 1) one or more checkpoint inhibitors (including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C). PD-L2 (such as, for example, rHIgM12B7). CTLA-4 (such as, for example, Ipilimumab (MDX-010), Tremelimumab (CP-675,206)), IDO, B7-H3 (such as, for example, MGA271, MGD009, omburtamab), B7-H4, T cell immunoreceptor with Ig and ITIM domains (TIGIT)(such as, for example BMS-986207, OMP-313M32, MK-7684, AB-154, ASP-8374, MTIG7192A, or PVSRIPO). CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep), 2) one or more transforming growth factor-β receptor 1 (TGFβR1) inhibitors (such as, for example, RepSox, SB525334, GW788388, Vactosertib, SD-208, Galunisertib, and/or LY3200882 or a vector encoding a CRISPR/Cas9 endonuclease integration system comprising a guide RNA (gRNA) that targets TGFβR1 gene; and 3) one or more bone morphogenic protein 4 (BMP4), BMP6, or BMP10 protein, and/or a BMP4, BMP6, or BMP10 agonists (such as, for example, the BMP4 agonists SB4, SJ000063181, SJ000291942, and/or SJ000370178). In one aspect, the method further comprises administering to the subject an antioxidant, including, but not limited to vitamin C.

For example, disclosed herein methods of treating, inhibiting, decreasing, reducing, ameliorating, and/or preventing an infectious disease wherein the microbial infection is a viral infection, wherein the viral infection comprises an infection of Herpes Simplex virus-1, Herpes Simplex virus-2. Varicella-Zoster virus. Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus Coronavirus (including, but not limited to spike or envelope proteins from avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus. Yellow fever virus, Zika virus, Ebola virus, Marburg virus. Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus. Simian Immunodeficiency virus, Human Immunodeficiency virus type-1, or Human Immunodeficiency virus type-2.

Also disclosed herein methods of treating, inhibiting, decreasing, reducing, ameliorating, and/or preventing an infectious disease wherein the microbial infection is a bacterial infection, wherein the bacterial infection comprises an infection of Mycobaterium tuberculosis, Mycobaterium bovis, Mycobaterium bovis strain BCG, BCG substrains, Mycobacterium avium, Mycobacterium intracellular, Mycobacterium africanum, Mycobaterium kansasii, Mycobaterium marinum, Mycobaterium ulcerans, Mycobaterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium dificile, other Clostridium species, Yersinia enterolitica, and other Yersinia species, and Mycoplasma species.

Also disclosed herein methods of treating, inhibiting, decreasing, reducing, ameliorating, and/or preventing an infectious disease wherein the microbial infection is a fungal infection, wherein the fungal infection comprises an infection of Candida albicans, Cryptococcus neoformans, Histoplasma capsulatum, Aspergillus fumigatus, Coccidiodes immitis. Paracoccidioides brasiliensis, Blastomyces dermitidis, Pneumocystis carinii, Penicillium marneffi, or Alternaria alternata.

Also disclosed herein methods of treating, inhibiting, decreasing, reducing, ameliorating, and/or preventing an infectious disease wherein the microbe causing the disease is a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

The components of the treatment regimen (i.e., the immune checkpoint inhibitor, the TGFβR1 inhibitor, and the BMP4 or BMP4 agonist) can be administered as a single composition or as multiple compositions. Where administered separately, the components can be administered concurrently or at any combination of different times. For example, the TGFβR1 inhibitor and the BMP4 or BMP4 agonist can be administered as a single composition or concurrently and prior to or after administration of the checkpoint inhibitor. Alternatively, the TGFβR1 inhibitor and the checkpoint inhibitor can be administered as a single composition or concurrently and prior to or after administration of the BMP4 or BMP4 agonist. In another aspect, the BMP4 or BMP4 agonist and the checkpoint inhibitor can be administered as a single composition or concurrently and prior to or after administration of the TGFβR1 inhibitor. In one aspect, the TGFβR1 inhibitor and the BMP4 or BMP4 agonist can be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 40, 45, 50, 55, 58, 59, 60, 61, 75, or 90 days 4, 5, 6, 7, 8, 9, 10, 11, or 12 months prior to or following administration of the checkpoint inhibitor.

Administration each component can occur a single time or multiple times for any duration as determined necessary by the practicing physician. For example, the the TGFβR1 inhibitor and the BMP4 or BMP4 agonist can be administered 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times with administrations occurring once every 1, 2, 3, 4, 5, 6, 7, 8, 9, 10, 11, 12, 13, 14, 15, 16, 17, 18, 19, 20, 21, 22, 23, 24, 30, 36, 42, 48 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, or 12 months.

### 2. Methods of Treating Cancer

The disclosed compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

In one aspect, disclosed herein are methods of treating, inhibiting, reducing, decreasing, ameliorating, and/or preventing a cancer and/or metastasis in a subject, the method comprising administering to the subject any of the treatment regimens disclosed herein. For example, disclosed herein are methods of treating, inhibiting, reducing, decreasing, ameliorating, and/or preventing a cancer and/or metastasis in a subject or methods of treating, inhibiting, reducing, decreasing, ameliorating, and/or preventing an infectious disease (such as, for example a viral infection, bacterial infection, parasitic infection, or fungal infection) in a subject, the method comprising administering to the subject 1) one or more checkpoint inhibitors (including, but are not limited to antibodies that block PD-1 (such as, for example, Nivolumab (BMS-936558 or MDX1106), pembrolizumab, CT-011, MK-3475), PD-L1 (such as, for example, alezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, BI754111, Sym022, FS118, MGD013, and Immutep), 2) one or more transforming growth factor-β receptor 1 (TGFβR1) inhibitors (such as, for example, RepSox, SB525334, GW788388, Vactosertib, SD-208, Galunisertib, and/or LY3200882 vector encoding a CRISPR/Cas9 endonuclease integration system comprising a guide RNA (gRNA) that targets TGFβR1 gene) and 3) one or more bone morphogenic protein 4 (BMP4), BMP6, or BMP10 protein, and/or a BMP4, BMP6, or BMP10 agonists (such as, for example, the BMP4 agonists SB4, SJ000063181, SJ000291942, and/or SJ000370178). In one aspect, the method further comprises administering to the subject an antioxidant, including, but not limited to vitamin C.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib). Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

The components of the treatment regimen (i.e., the immune checkpoint inhibitor, the TGFβR1 inhibitor, and the BMP4 or BMP4 agonist) can be administered as a single composition or as multiple compositions. Where administered separately, the components can be administered concurrently or at any combination of different times. For example, the TGFβR1 inhibitor and the BMP4 or BMP4 agonist can be administered as a single composition or concurrently and prior to or after administration of the checkpoint inhibitor. Alternatively, the TGFβR1 inhibitor and the checkpoint inhibitor can be administered as a single composition or concurrently and prior to or after administration of the BMP4 or BMP4 agonist. In another aspect, the BMP4 or BMP4 agonist and the checkpoint inhibitor can be administered as a single composition or concurrently and prior to or after administration of the TGFβR1 inhibitor. In one aspect, the TGFβR1 inhibitor and the BMP4 or BMP4 agonist can be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 40, 45, 50, 55, 58, 59, 60, 61, 75, or 90 days 4, 5, 6, 7, 8, 9, 10, 11, or 12 months prior to or following administration of the checkpoint inhibitor.

Administration each component can occur a single time or multiple times for any duration as determined necessary by the practicing physician. For example, the the TGFβR1 inhibitor and the BMP4 or BMP4 agonist can be administered 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times with administrations occurring once every 1, 2, 3, 4, 5, 6, 7, 8, 9, 10, 11, 12, 13, 14, 15, 16, 17, 18, 19, 20, 21, 22, 23, 24, 30, 36, 42, 48 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, or 12 months.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

We were the first to show that de novo DNA methylation programs, mediated by the Dnmt3a enzyme, are causally linked to T cell silencing, and enforce full exhaustion during chronic virus infection or cancer. More importantly, we discovered that T cell-intrinsic epigenetic programs restrain the efficacy of ICB therapy, perhaps explaining ICB failure in cancer patients whose anti-tumor T cells are fully-exhausted. However, master regulators of DNA methylation programs that govern T cell function versus exhaustion remain largely unknown. We propose a unique approach to reverse the fully-exhausted state by targeting T cell-intrinsic epigenetic programs. Based on our data, we hypothesize that TGFβ1 signaling regulates de novo DNA methylation in persistently stimulated CD8 T cells, skewing them toward a fully-exhausted state, which promotes their resistance to ICB therapy.

a) Investigate the role of TGFβ1 signals in regulating de novo epigenetic programs and in curbing the rejuvenation potential of exhausted T cells.

Our data revealed TGFβ1 signaling as the most significant pathway that orchestrates DNA methylation, chromatin accessibility, and transcriptional changes in exhausted CD8 T cells from mice or humans. To test our hypothesis that TGFβ1 signaling promotes exhaustion-associated epigenetic programming, whereas augmentation of BMP-signaling sustains the pool of the ICB-responsive, partially-exhausted T cells, we can employ cutting-edge approaches, such as CRISPR-Cas9 gene-editing or retroviral (RV) transduction in TCR-transgenic P14 CD8 T cells. We can use a novel in-vitro model and complementary pre-clinical models of T cell exhaustion, including glioma and chronic LCMV infection to: 1a) determine whether SMAD2/3-TGFβ1 signaling drives CD8 T cell epigenomes toward the fully-exhausted state; 1b) test whether negative regulators of TGFβ signaling prevent the establishment of exhaustion-specific epigenetic signatures; 1c) determine whether BMP-signals counteract TGFβ-mediated epigenetic programming in exhausted T cells; 1d) test targeting TGFβ1-signaling and/or augmenting BMP-signaling to enhance responses to ICB therapy by preserving the pool of partially-exhausted T cells.

### b) Research

In the last 2 decades, advancements in our basic understanding of T cell biology led to breakthrough discoveries for treating cancer and chronic virus infections. Specifically, we learned to exploit the selective killing power of CD8 T cells through either rejuvenating exhausted cells with ICB therapy, or by re-directing their specificities with chimeric antigen receptors (CAR) to attack cancer cells expressing tumor antigens. Yet, terminal T cell-exhaustion remains a major obstacle to these immunotherapies. During persistent TCR (T cell receptor) stimulation, cytotoxic T cells progressively lose their effector functions, transitioning through a partially-exhausted state (also defined as “stem-like” or “progenitor”), which remains ICB-responsive, before they become fully exhausted and refractory to current checkpoint blockade protocols. This progression to full exhaustion can explain why many patients fail to mount durable responses to ICB therapy. In addition, the therapeutic efficacy and in-vivo persistence of CAR T cells is limited, in part, due to their progression toward exhaustion. A recent study has also reported that PD-1 blockade treatment did not enhance the efficacy of CAR T cells for treatment of neuroblastoma, implying that these cells acquire a fully-exhausted, ICB-nonresponsive state. Thus, a better understanding of the molecular mechanisms underlying terminal T cell exhaustion is crucial to enhance the efficacy of T cell immunotherapies.

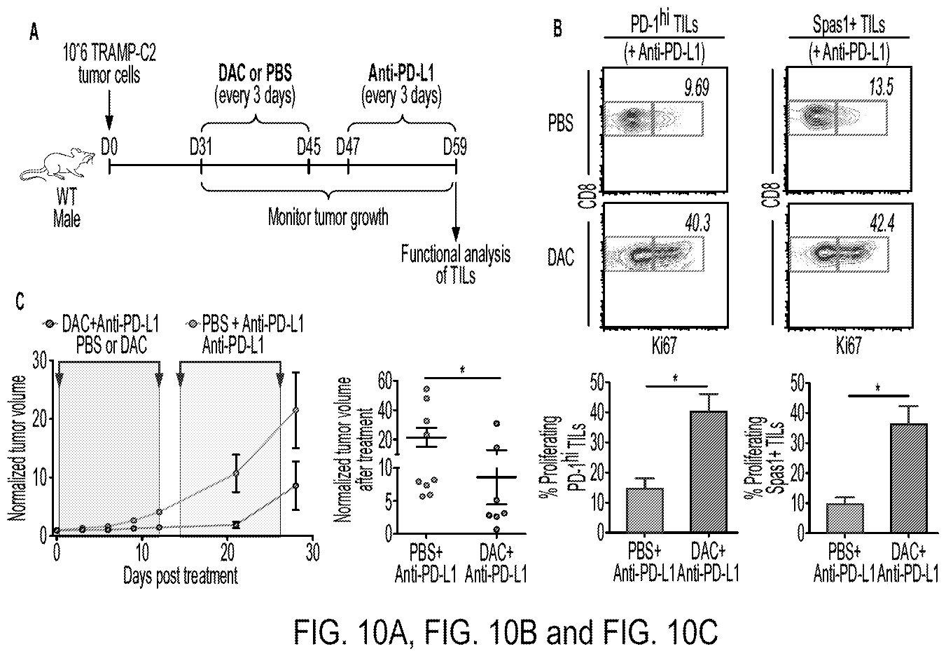

Epigenetic programming is a fundamental mechanism that regulates gene expression and cell fate commitment. De novo DNA methyltransferase 3a (Dnmt3a) enforces terminal T cell exhaustion through the epigenetic silencing of key genes regulating T cell functions. Importantly, we discovered a novel approach to enhance the efficacy of ICB therapy through targeted inhibition of T cell-intrinsic de novo DNA methylation. We found that genetic deletion of Dnmt3a in activated CD8 T cells or therapeutic erasure of DNA methylation programs by decitabine treatment synergize with ICB therapy to rescue the response of exhausted T cells. (FIG. 10). Yet, decitabine is a non-selective, highly cytotoxic drug that has adverse effects on other immune cells. In addition, Dnmt3a mutations are common in hematological malignancies, many of which are linked to pre-leukemic dedifferentiation. Thus, one must be cautious about targeting all Dnmt3a-mediated epigenetic programs, since this can be advantageous for tumor progression. Instead, a major goal of our studies is to identify factors that inhibit the specific epigenetic programs that drive progression of CD8 T cells to full exhaustion, and to target these factors for enhancing the efficacy of T cell immunotherapies.

CD8 T cells undergo distinct epigenetic changes (DNA methylation and chromatin accessibility), coupled to the development of exhaustion. While these epigenetic maps provide a mechanistic basis for the maintenance of gene expression programs in exhausted T cells, the following questions represent major gaps in our current understanding of T cell exhaustion during chronic infections or cancer: (1) How are these epigenetic changes acquired and maintained in exhausted T cells? (2) What are the upstream signals that regulate the specificity of de novo DNA methylation programs in exhausted versus functional T cells?(3) Can we reverse epigenetic programming in exhausted T cells to the functional state while avoiding transformation? Bridging these gaps allows us to identify and target factors that apply “epigenetic brakes” to CD8 T cell function. The HMG-box transcription factor TOX mediates wide-spread chromatin accessibility changes in effector CD8 T cells, which are critical for the generation and maintenance of a partially-exhausted state during chronic infections or cancer. Importantly, genetic deletion of TOX initially enhanced effector function in CD8 T cells, but eventually induced a massive quantitative loss of exhausted T cells. These findings indicate that targeting TOX may not be an effective therapeutic approach, given its counteractive functions in exhaustion and survival. Instead, our scientific premise and data (described below) reveal novel upstream regulators of exhaustion-associated epigenetic programs and indicate innovative approaches to rescue both function and sternness of fully-exhausted CD8 T cells.

Full exhaustion is a major challenge to T cell immunotherapies. Therefore, it is increasingly important to identify cell-intrinsic barriers for rejuvenation of fully-exhausted CD8 T cells during chronic virus infections or cancer. We propose a unique approach to reverse the fully-exhausted state of T cells by targeting epigenetic programs that are T cell-intrinsic or are imprinted by the microenvironmental signals. The findings that provide the scientific premise of our proposal are outlined below.

### c) De Novo DNA Methylation is Essential for Establishing CD8 T Cell Exhaustion.

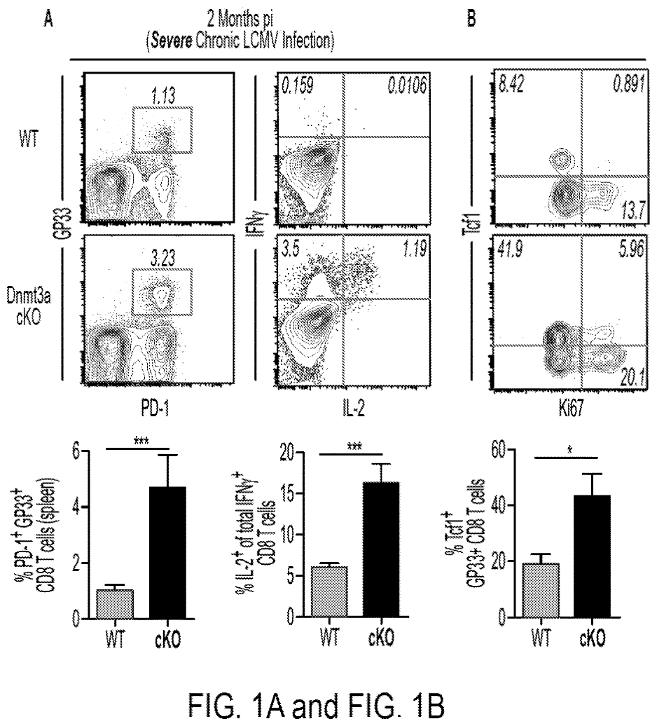

We recently discovered that de novo DNA methylation-mediated by the Dnmt3a enzyme-plays a critical role in establishing T cell exhaustion. Conditional deletion of Dnmt3a (Dnmt3a cKO) in activated CD8 T cells blocks their progression toward the full exhaustion state. Furthermore. Dnmt3a-deficient antigen-specific CD8 T cells are exhaustion-resistant during lifelong chronic lymphocytic choriomeningitis virus (LCMV) infection—an animal model of severe T cell exhaustion. At the mechanistic level, we found that antigen-specific cKO CD8 T cells were maintained at higher numbers in lymphoid and non-lymphoid tissues, and retained their capacity to recall effector cytokines (e.g., IFNγ, IL-2) relative to the wild type (WT) CD8 T cells, despite prolonged TCR stimulation and high PD-1 expression (FIG. 1A). In addition, Dnmt3a-deficient CD8 T cells continued to express higher levels of stemness-associated transcription factors, such as Tcf1 and Lef1 (FIG. 1B). These data established de novo DNA methylation programming as a novel, cell-intrinsic mechanism that is causally linked to T cell exhaustion.

### d) De Novo DNA Methylation Enforces the Silencing of Effector and Stemness Programs in Exhausted T Cells.

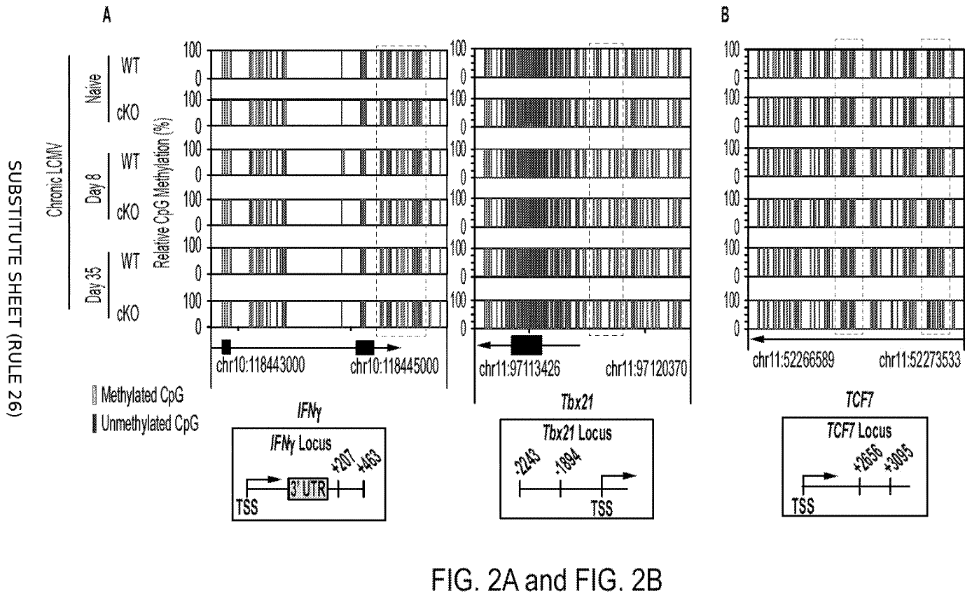

Recent studies have shown that exhaustion-associated transcriptional programs can be maintained in the absence of antigen, indicating an underlying cell-intrinsic mechanism. To better understand the heritable nature of exhaustion-specific transcriptional programs, we profiled whole-genome DNA methylation changes in antigen-specific WT and Dnmt3a CD8 cKO T cells during acute and chronic virus infections on day 35 post-infection (p.i). We found that: (i) WT CD8 T cells acquired ˜980 and ˜1200 newly methylated regions during the naïve-to-effector and effector-to-exhausted transitions, respectively, and (ii) many of these differentially methylated regions (DMRs) were acquired in genes that regulate CD8 T cell's function and self-renewal, such as IFNg, Tbx21 (encoding T-bet), and Tcf7 (encoding Tcf1) (FIG. 2A, 2B). Taken together, these findings indicate that de novo DNA methylation serves as a T cell-intrinsic mechanism, which enforces the silencing of effector function and sternness programs in exhausted T cells.

### e) TOX Remodels the Open Chromatin Landscape of CD8 T Cells Toward Exhaustion.

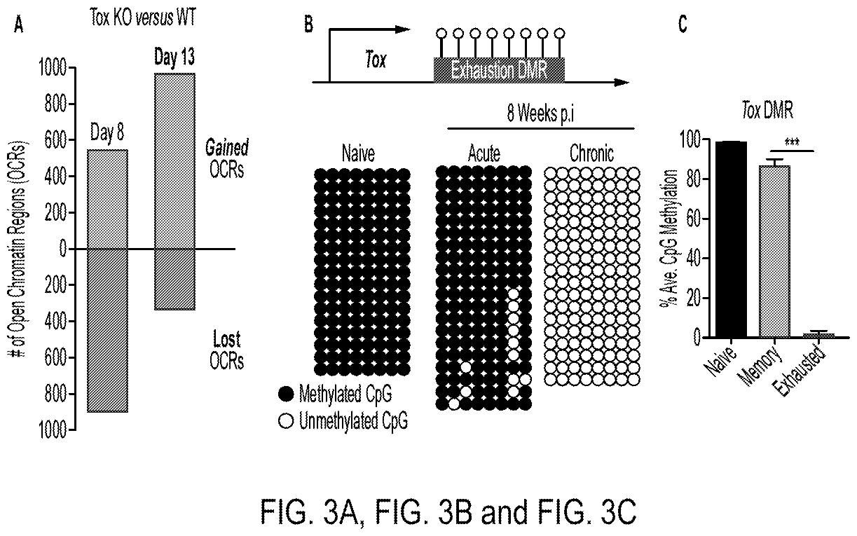

The transcription factor TOX reprograms the open chromatin landscape of effector CD8 T cells toward the generation of partially-exhausted T cells (FIG. 3A). We also discovered a unique DNA demethylation program in the TOX locus, that enforces the sustained upregulation of TOX in exhausted CD8 T cells (FIGS. 3B and 3C). However, targeting TOX revealed opposing outcomes (enhanced effector function, but impaired survival of exhausted T cells), in essence, negating it as a therapeutic target.

### f) De Novo DNA Methylation Reshapes the Open Chromatin Landscape of Exhausted CD8 T Cells.

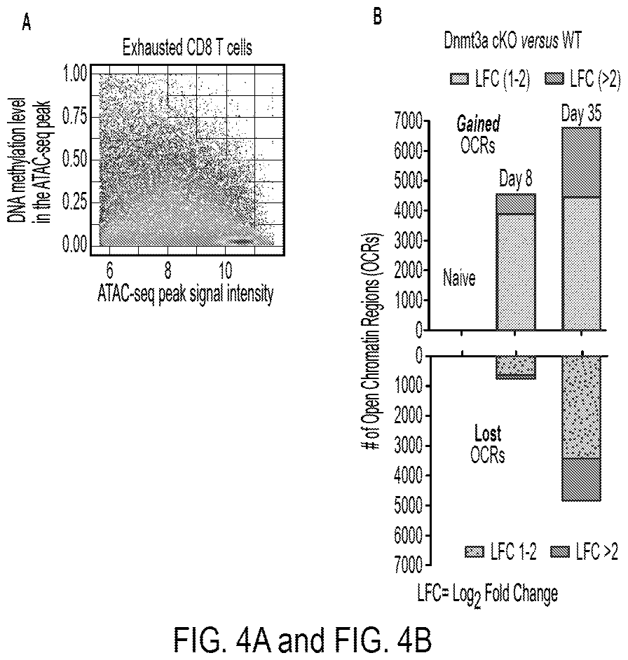

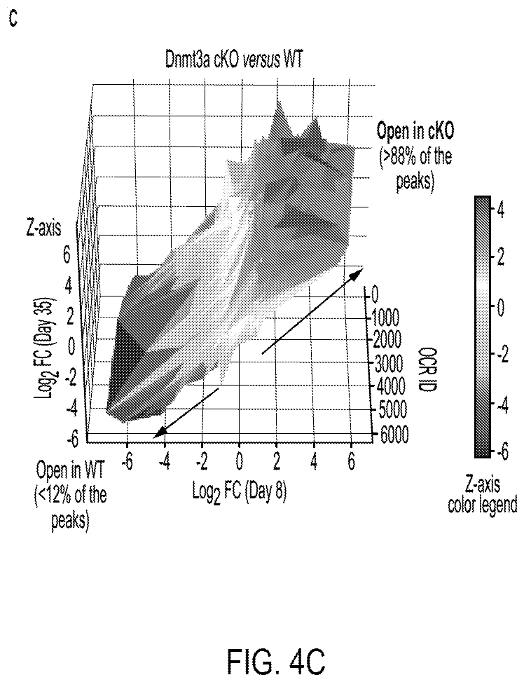

We and others have recently reported that exhausted CD8 T cells acquire distinct chromatin accessibility and DNA methylation changes during chronic virus infection or cancer. Yet, it is not clear how this open chromatin landscape is maintained during exhaustion, or the impact of specific chromatin accessibility changes on the maintenance of exhausted T cell biology. We sought to address these questions by determining if de novo DNA methylation regulates chromatin accessibility changes coupled to T cell exhaustion. We profiled the open chromatin landscape in antigen-specific WT and Dnmt3a cKO CD8 T cells during chronic LCMV infection. We also tracked differences in chromatin accessibility at the open chromatin regions (OCRs) (x-axis) detected on both days 8 (v-axis) and 35 (z-axis) p.i. We found that: (1) DNA methylation and chromatin accessibility changes are inversely linked in exhausted CD8 T cells (FIG. 4A), with >60% of exhaustion-associated chromatin accessibility changes regulated by de novo DNA methylation; (2) Dnmt3a-deficient CD8 T cells retain chromatin accessibility at thousands of OCRs at both effector (day 8) and exhaustion (day 35) timepoints relative to WT CD8 T cells (FIG. 4B). Intriguingly, cKO CD8 T cells also have reduced chromatin accessibility in some regions, particularly during the exhaustion phase (FIG. 4B). (3) The majority of exhaustion-associated, Dnmt3a-regulated accessibility changes are acquired after the effector stage of T cell differentiation (FIG. 4C). We also analyzed transcription factor-binding motifs enriched within the Dnmt3a-mediated DMRs and OCRs in exhausted T cells, and found that de novo DNA methylation plays a major role in cis- and trans-regulation of the chromatin remodeling at exhaustion-specific transcriptional circuits. Collectively, our findings indicate that de novo DNA methylation, in part, can regulate and shape the unique open chromatin landscape found in exhausted CD8 T cells.

DNA methylation programming is a T cell-intrinsic mechanism that establishes T cell exhaustion. Our work showed that targeting T cell-intrinsic DNA methylation synergizes the response to ICB therapy. However, the key players and therapeutic targets that initiate or maintain de novo DNA methylation programs in exhausted T cells are completely unknown. TGFβ1 signaling drives de novo DNA methylation in CD8 T cells, skewing them toward a fully-exhausted state. This hypothesis is based on three observations: (1) Our integrated analysis revealed TGFβ1 signaling as the most significant pathway that regulates changes in DNA methylation, chromatin accessibility, and transcriptional programs in exhausted CD8 T cells from mice and humans. (2) Loci that encode cellular inhibitors of SMAD2/3-dependent TGFβ signaling acquire DNA methylation, coupled with reduced chromatin accessibility and transcription in fully-exhausted CD8 T cells. (3) Therapeutic blockade of TGFβ1 signaling prevents T cell exhaustion and reprograms the expression of CD62L.

### g) Investigate the Role of TGFβ Signals in Regulating De Novo Epigenetic Programs and in Curbing the Rejuvenation Potential of Exhausted T Cells.

Rationale: While our data highlight the significant role of Dnmt3a enzyme in mediating exhaustion-associated programs in CD8 T cells, the upstream regulators that orchestrate these epigenetic changes are largely unknown. Persistent TCR stimulation is thought to be a key driver of T cell exhaustion during chronic virus infection or in tumor settings². Yet, such as beta cell-specific CD8 T cells in type I diabetic patients, preserve effector function and stemness features, despite their prolonged exposure to auto-antigens. Such dichotomous outcomes of persistent antigen exposure indicate an important role of microenvironmental signals in modulating antigen-specific CD8 T cell function. Therefore, we hypothesize that the nature and/or duration of microenvironmental cues orchestrate epigenetic changes downstream of persistent TCR stimulation in exhausted T cells.

Earlier studies have shown that TGFβ1 signaling directly suppresses the cytotoxic function and proliferation of CD8 T cells during cancer or chronic infections. However, the underlying molecular mechanisms that induce and/or stabilize this suppressive function remain poorly understood. Most studies have focused on the impact of TGFβ signals during the priming phase of naïve CD8 T cells. Importantly, the effect of TGFβ signaling on the epigenetic programming of effector versus exhausted CD8 T cells remains largely unknown. Our data revealed TGFβ1 signaling as the most significant pathway that orchestrates epigenetic and transcriptional changes within exhausted CD8 T cells. Therefore, we propose to determine whether TGFβ1 signaling enforces epigenetic programming within persistently stimulated CD8 T cells, driving them toward the fully-exhausted state and, in complementary studies, define the role of BMP-signaling as a mechanism to maintain a partially-exhausted phenotype.

h) TGFβ1 Signaling is the Most Significant Pathway that Orchestrates Epigenetic and Transcriptional Changes in Exhausted CD8 T Cells from Mice or Humans.

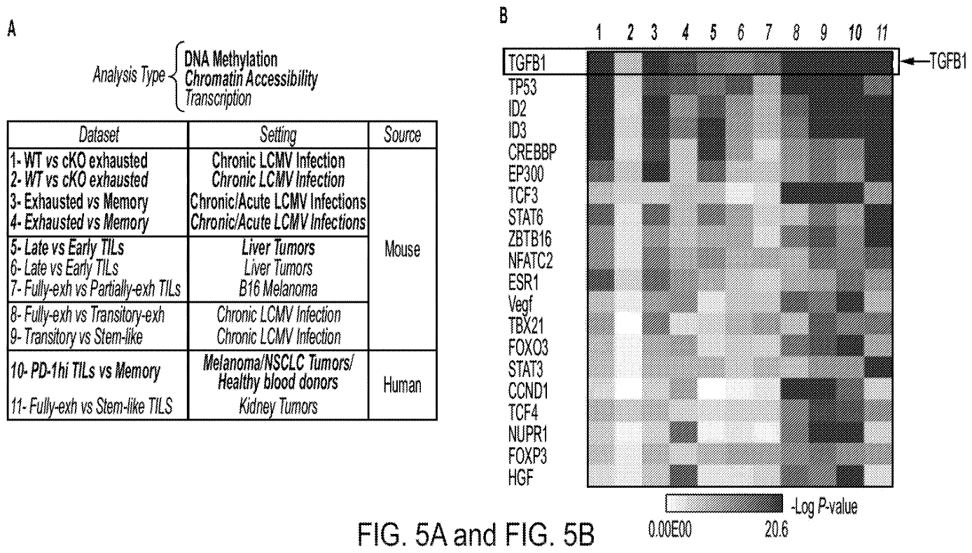

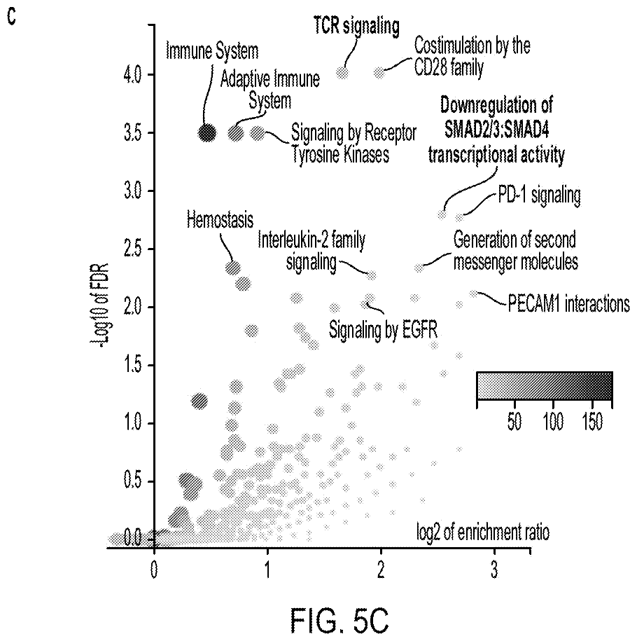

To identify and prioritize the conserved upstream regulators of exhaustion-associated epigenetic programs during chronic virus infections and cancer, we performed integrative analyses of DNA methylation, chromatin accessibility, and transcriptional changes in exhausted versus functional or partially-exhausted CD8 T cells. We complied 11 datasets from mouse and human CD8 T cell subsets. Multi-IPA analysis of epigenetic and transcriptional changes used the listed comparisons in FIG. 5A. Our integrative analysis revealed TGFβ1 as the most significant upstream regulator of epigenetic and transcriptional changes in fully-exhausted T cells from both mice and humans (FIG. 5B).

TGFβ1 is a highly conserved cytokine with pleiotropic biological functions and downstream signaling pathways that are orchestrated through context-dependent interactions of SMADs. To gain insights into the TGFβ signaling pathway that drives full-exhaustion in CD8 T cells, we performed Reactome Pathway Enrichment analysis for Dnmt3a-target genes that gained chromatin accessibility or were upregulated in chronically stimulated Dnmt3a-deficient versus exhausted WT CD8 T cells. As shown in FIG. 5C, Smad2/3-dependent TGFβ signaling is significantly downregulated in the exhaustion-resistant, Dnmt3a-deficient CD8 T cells. Together, our data indicate a conserved role for SMAD2/3-dependent TGFβ signaling in driving the T cell-epigenome and transcriptome toward a fully-exhausted state during chronic virus infections or cancer.

### i) Negative Regulators of Smad2/3-TGFβ Signaling are Progressively Downregulated in CD8 T Cells During the Progression Toward Full Exhaustion.

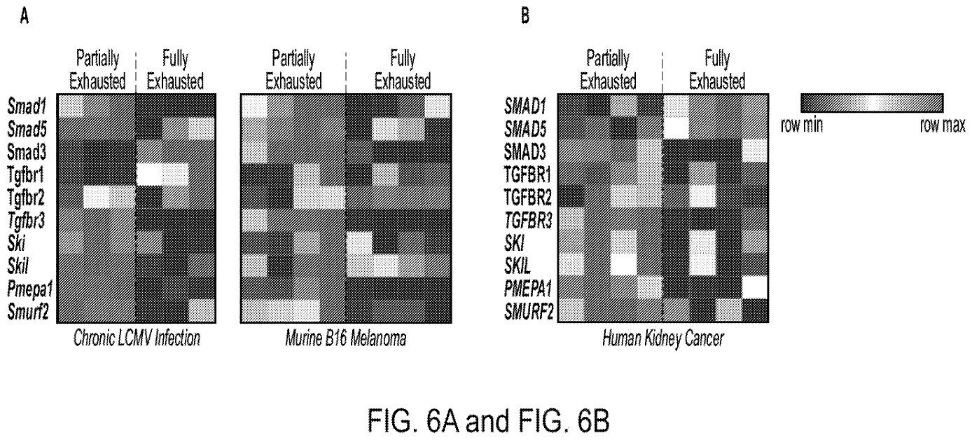

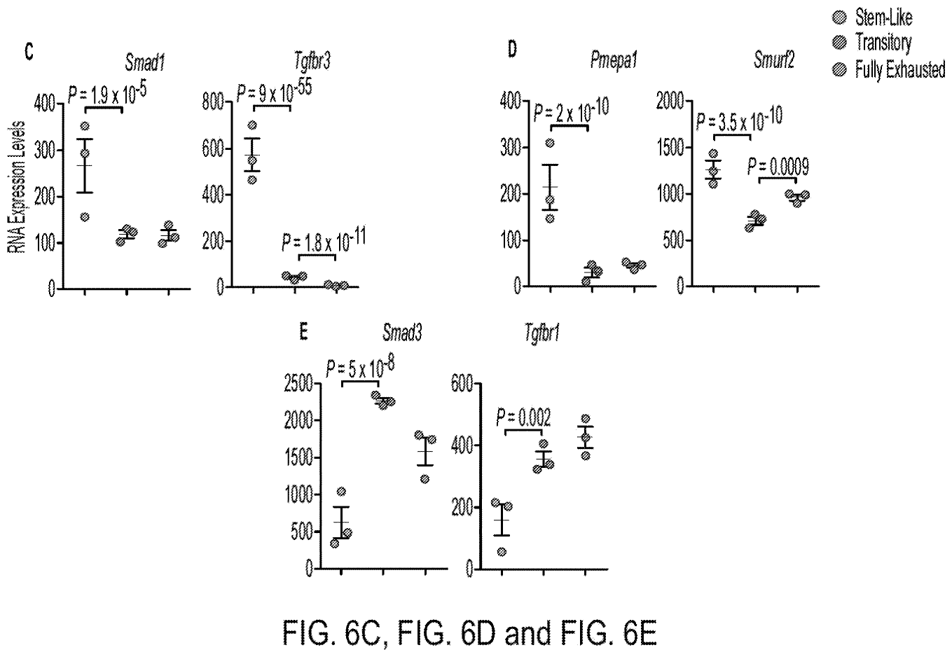

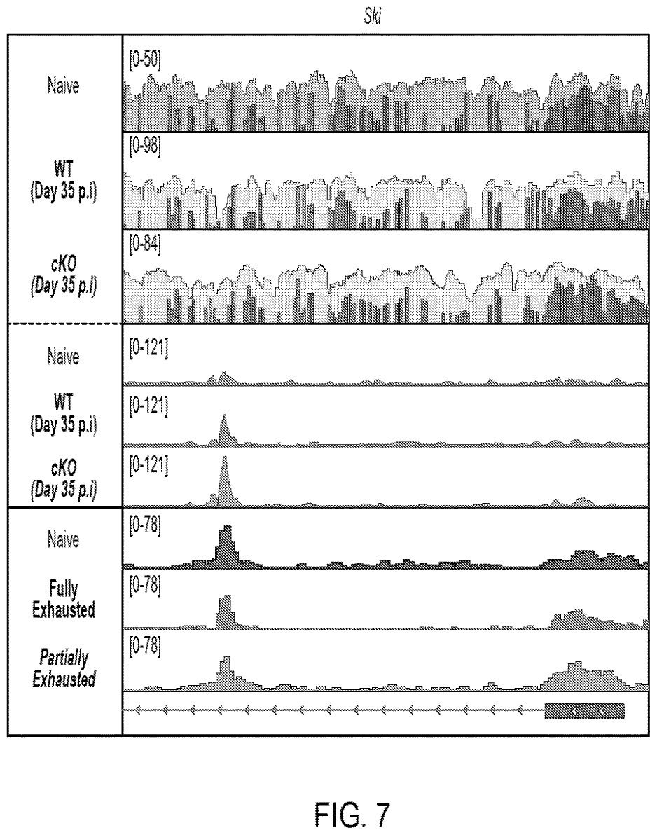

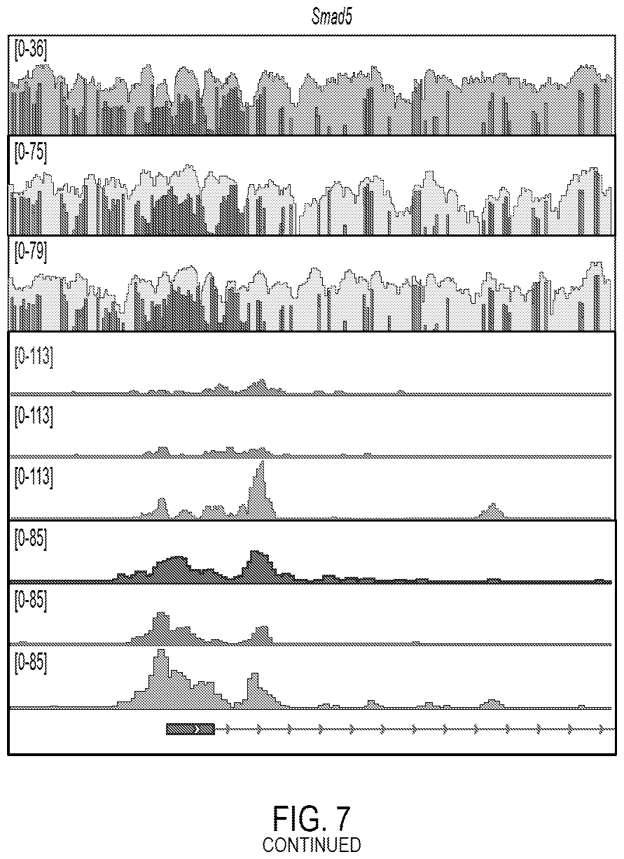

To better understand whether TGFβ signaling is differentially regulated in exhausted T cells, we investigated the transcriptional and epigenetic changes in its key transducers and rheostats. We found that partially-exhausted CD8 T cells retain higher expression levels of Smad1/5, Tgfbr3, in addition to negative regulators of SMAD2/3-signaling, such as SKI, SKIL (SKI-like protein), PMEPA1, and/or SMURF2, during chronic infections or cancer (FIG. 6A, 6B). Conversely, fully-exhausted, virus-specific CD8 T cells express higher levels of Smad3 (FIG. 6A). A recent study reported a “Transitory” effector-like subset of CD8 T cells (CD101^{-ve}) within the fully-exhausted T cell population (PD-1+ Tim-3+) during chronic virus infection. The “Transitory” cells retain intermediate levels of effector function and stemness, and quickly give rise to the fully-exhausted population (CD101+). Yet, we found that the “Transitory” subset expresses higher RNA levels of Smad3 and low RNA levels of Smad1, Tgfbr3, Pmepa1, and Smurf2, a pattern similar to the fully-exhausted cells (FIGS. 6C, 6D, and 6E). We next examined DNA methylation and chromatin accessibility datasets, and found that some of the TGFβ1 attenuators acquire de novo DNA methylation programs, coupled with reduced chromatin accessibility in fully-exhausted WT CD8 T cells (FIG. 7). Collectively, our data indicate that partially-exhausted T cells favor Smad1/5-dependent BMP-signaling. This preferential signaling can be explained, in part, by the higher expression of TGFβ1-negative regulators that help preserve the effector function and stemness programs during persistent TCR stimulation. It also implies an opposite role of Smad1/5- versus Smad2/3-signaling pathways in regulating the progression toward the full-exhaustion state.

### j) Therapeutic Blockade of TGFβ1-Signaling Inhibits Functional Exhaustion and Promotes Survival of Persistently Stimulated CD8 T Cells In Vitro.

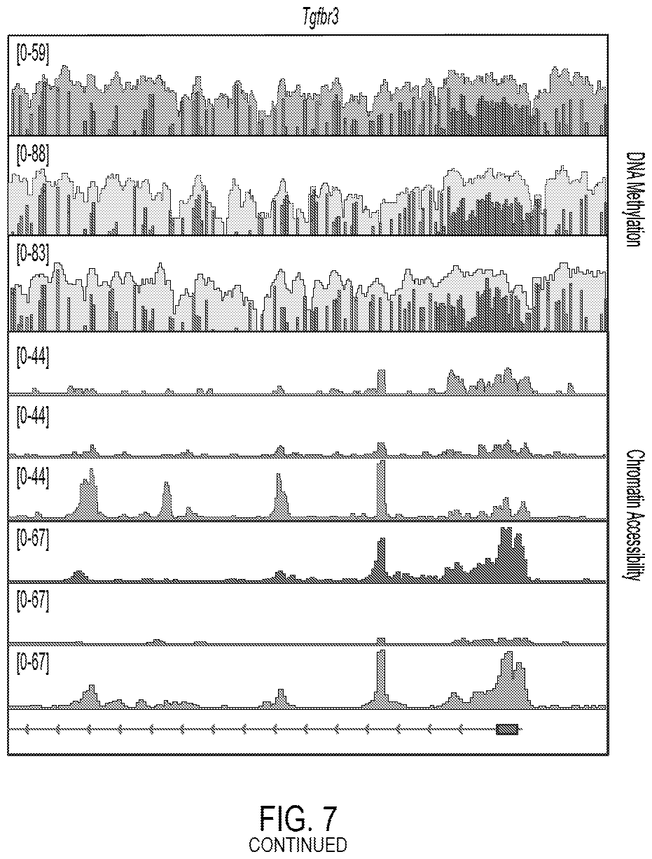

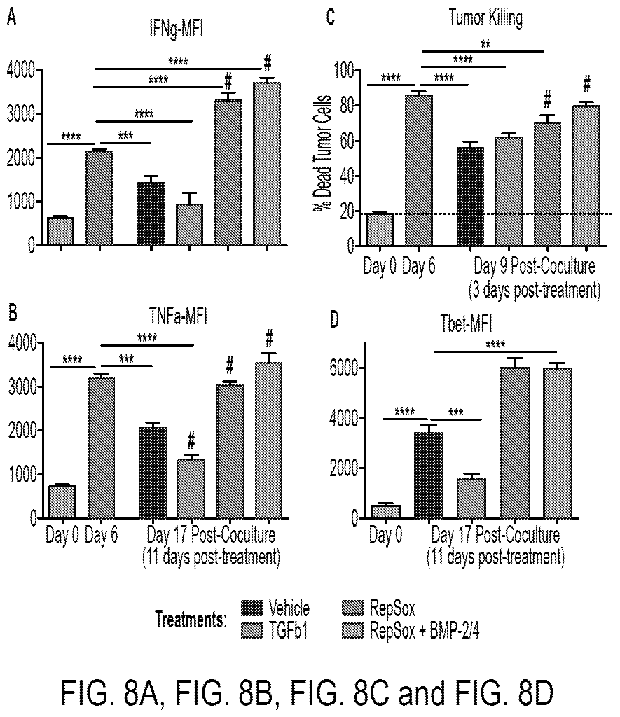

To study their epigenetic (re)programming, we developed a novel in-vitro model of CD8 T cell exhaustion. First, we optimized conditions for in-vitro co-culture of gp33-expressing murine brain tumor cells (CT2A-gp33) and TCR transgenic gp33-specific CD8 T cells (P14 cells). We were able to enforce differentiation of anti-tumor CD8 T cells under their persistent TCR stimulation by repeated exposure to the tumor antigen and tumor-derived factors, such as TGFβ1. Initially, we found that naïve P14 CD8 T cells can differentiate into highly cytotoxic, effector T cells within 6-8 days in vitro (blue bar in FIGS. 8A, 8B, and 8C). However, with prolonged exposure to the tumor antigen, the effector CD8 T cells progressively lose cytokine production and tumor killing activity (FIGS. 8A, 8B, and 8C). These data underscore the utility of our in-vitro model as a first-line platform to study T cell dysfunction or exhaustion under persistent antigen exposure, prior to moving in vivo.

To assess the impact of TGFβ1 signaling on the progression of CD8 T cells toward dysfunction, we treated co-cultures with TGFβ1 or RepSox (also known as E-616452) (2-(3-(6-Methylpyridine-2-yl)-1H-pyrazol-4-yl)-1,5-naphthyridine) as shown in the formula:

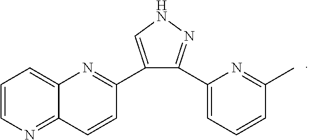 [SMILES: Cc1cccc(-c2n[nH]cc2-c2ccc3ncccc3n2)n1]

RepSox is a potent selective inhibitor of the TGFβ type 1 receptor (ALK5)—for 11 days after effector differentiation was complete (day 6). We found that persistent TGFβ1 treatment accelerated the impairment to production of IFNγ and TNFα (FIGS. 8A, and 8B) and repressed expression of the T-bet transcription factor—a key regulator of memory T cell function—and Granzyme B (FIG. 8D). Conversely, RepSox treatment not only preserved tumor killing activity, but also boosted the effector function of persistently stimulated P14 cells, indicated by higher expression of IFNγ, T-bet and Gzmb effector molecules relative to Day 6-effector CD8 T cells (FIG. 8).

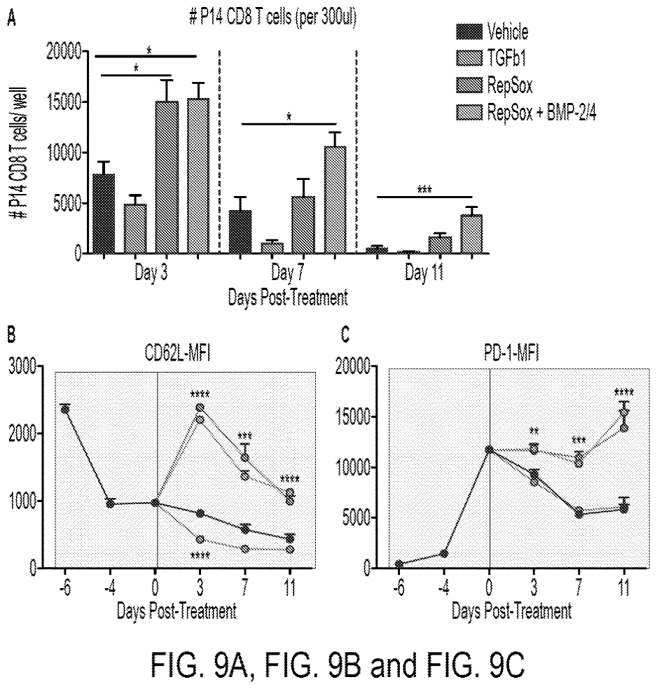

We also examined the impact of BMP-2/4 signaling on RepSox-treated P14 CD8 T cells. Although the addition of BMP-2/4 cytokines to RepSox treatment did not induce further improvement in effector functions, combined treatment rescued significantly higher numbers of persistently stimulated anti-tumor CD8 T cells (FIG. 9A). Moreover, RepSox treatment of P14 CD8 T cells restored CD62L (L-selectin) expression, a key lymphoid tissue-homing molecule that is highly expressed on naïve or central-memory CD8 T cells, and maintained high levels of PD-1 (FIGS. 9B and 9C). These data indicate that TGFβ1 blockade can epigenetically reprogram the expression of CD62L, and possibly other stemness/lymphoid tissue homing molecules, which are silenced during effector CD8 T cell differentiation. Taken together, our findings indicate that TGFβ1-signaling not only accelerates the development of T cell dysfunction, but also maintains silencing of lymphoid homing markers on CD8 T cells. Importantly, our findings support the feasibility of using this novel in-vitro model as a tractable tool that can guide our in-vivo work by providing first-line mechanistic studies.

### k) Determine Whether SMAD2/3-TGFβ1 Signaling Drives CD8 T Cell Epigenomes Toward the Fully-Exhausted State.

Our data indicate that SMAD2/3-TGFβ1 signaling plays a key role in regulating the epigenetic and transcriptional changes in fully-exhausted CD8 T cells. To rigorously test and define the kinetics of this pathway for signal-induced exhaustion, we can genetically ablate the positive regulators of TGFβ1 signaling, Tgfbr1 and Smad3, in P14 CD8 T cells using a CRISPR-Cas9 protocol optimized in our lab for these primary cells (editing efficiency ˜70-80%). These edited P14 cells can be used for our in-vitro and in-vivo experiments as described below. To determine the impact of TGFβ1 signaling on exhaustion of pre- versus post-effector CD8 T cells, we can engineer P14 cells after 1 day (early activation phase) or 6 days (effector phase) of co-culture with CT2A-gp33 tumor cells. Genetically edited P14 cells can be maintained with persistent exposure to the tumor antigen in the presence or absence of exogenous TGFβ1. After total 16-20 days of co-culture with tumor cells, we can isolate edited or control P14 cells and assess their effector functions, quantifying cytokine production (e.g., IFNγ, TNFα, IL-2) after ex vivo stimulation with the gp33 peptide, and the levels of T-bet, Granzyme B. and Ki-67 by multi-color flow cytometry. As a complementary metric for exhaustion, we can measure expression of stemness-related molecules Tcf1 and CD62L in P14 cells by flow cytometry. The work has revealed DMRs and OCRs signatures that are distinct among functional, partially-exhausted, and fully-exhausted CD8 T cell subsets. Accordingly, we can examine levels of DNA methylation and chromatin accessibility in key genes that regulate effector (e.g., IFNg, Tbx21, Myc) or sternness (Tcf7, Lef1, CD62L) programs in edited or control P14 cells isolated from the above experiments. These in-vitro experiments can inform our mechanistic in-vivo studies related to the role and timing of SMAD2/3-TGFβ1 signaling in driving exhaustion-associated epigenetic programs.

To determine the impact of TGFβ1 signaling on epigenetic programming and functionality of T cells during chronic virus infection or cancer, we can adoptively transfer ˜5-10K control (Thy1.1+/1.2+) or genetically edited (Thy1.1+) P14 cells into chronically LCMV-infected C57BL/6 mice (day −1), or into CT2A-gp33 tumor-bearing Rag1 KO mice (>day 8 post-subcutaneous implantation of tumor cells). We can longitudinally track the function and phenotype of adoptively transferred P14 cells during the effector and chronic stages of LCMV infection. In the latter experiments, we can monitor tumor growth and assess the phenotype and function of P14 cells in tumors, draining lymph nodes (dLNs), and spleens at indicated time points (1-4 weeks) after the adoptive transfer.

To profile global epigenetic changes downstream SMAD2/3-TGFβ1 signaling in CD8 T cells, we can employ state-of-the-art technologies, including MeSMLR-seq (Methyltransferase treatment followed by Single-Molecule Long-Read sequencing). This approach contains two main steps: (1) GpC methyltransferase treatment of the nuclei, that are isolated from P14 cells, to convert cytosine to 5mC at GpC sites in naked linker DNA and open chromatin; and (2) Oxford Nanopore Technologies (ONT) sequencing to detect the 5mC profile that is subsequently used to identify nucleosome occupancy and chromatin accessibility, in addition to the natural DNA methylation at the CpG sites. This novel approach provides unique advantages over other epigenetic sequencing platforms: (i) The methylated cytosines are directly detected with individual base resolution at the single-DNA molecule level, (ii) Native DNA molecules can be directly sequenced without PCR amplifications and unintended sequencing bias, and (iii) the ONT allows sequencing of ultra-long reads (up to 2 Mb). Thus, this novel approach allows us to measure changes in DNA methylation, chromatin accessibility, and nucleosome positioning simultaneously, on FACS-purified P14 cells. Applying this approach can reveal the spectrum of TGFβ1-mediated changes to the exhausted T cell epigenomes at large genomic scale.

The data support a key role for TGFβ1 signaling in regulating the epigenetic progression toward terminal T cell exhaustion. Therefore. Tgfbr1- or Smad3-edited P14 cells can have improved effector function (e.g., IFNγ, IL-2, TNFα, Gzmb, T-bet) and/or higher expression of sternness markers (e.g., Tcf1, CD62L) under settings of persistent antigen exposure.

We have optimized the library preparation and sequencing of relatively small numbers of CD8 T cells using an ONT sequencer in our laboratory. Epigenetic profiling of genetically edited P14 cells at different timepoints can reveal the dynamics and hierarchy of TGFβ-mediated global changes to the T cell epigenome as they progress to the fully-exhausted state. As an alternative approach, we can perform both WGBS and ATAC-sequencing on P14 cells, to profile the changes in DNA methylation and open chromatin landscapes, respectively. Although WGBS and ATAC-seq do not reveal the epigenetic heterogeneity at the single-DNA molecule level as in MeSMLR-seq, they can accurately profile DNA methylation and chromatin accessibility changes downstream of TGFβ1 signaling in exhausted T cells.

Our data indicate a conserved role for TGFβ1 signaling in the regulation of T cell exhaustion. Yet, it remains possible that the pathway has different effects during chronic infections and cancer, which is precisely why we can use models for both. Another possibility is that P14 cells can be exposed to variable levels of TGFβ1 and other extrinsic cues within the tumor microenvironment (TME), depending on levels of tumor burdens in different animals. As an alternative approach, we can co-adoptively transfer both control (Thy1.1+/1.2+) and genetically edited (Thy1.1+) P14 CD8 T cells into the same CT2A-gp33 tumor-bearing Rag1 KO mice, to control for the effect of differences in the levels of TGFβ1 and other TME signals. We have not precluded the possibility that TGFβ1 signaling regulates exhaustion-associated epigenetic programs via SMAD2/3-independent pathways. As an alternative, unbiased approach, we can perform CRISPR-screening for all TGFβ1 regulators in P14 cells, including MEK1/2, ERK1/2, MKK4.

### 1) Test Whether Negative Regulators of TGFβ Signaling Prevent the Establishment of Exhaustion-Specific Epigenetic Signatures.

The data demonstrate that, relative to partially-exhausted cells, fully-exhausted CD8 T cells have reduced expression of cellular factors that attenuate TGFβ1 signaling, including SKI, SKIL, and PMEPA1. To determine whether these factors also prevent imprinting of exhaustion-associated epigenetic programs, we can induce ectopic expression of Ski or Skil within effector P14 cells using a tet-responsive retroviral (RV) transduction system. We can follow an optimized transduction protocol that improved the cell recovery and frequency of RV-transduced P14 cells. We can then analyze phenotypic, functional, and epigenetic changes in persistently stimulated P14 cells in our in-vitro model. We can track the function and survival of adoptively transferred OE P14 cells during chronic LCMV infection or CT2A-gp33 tumors, and profile the epigenetic changes at the exhaustion-defining epigenetic programs.

The enforced expression of Ski or Skil in CD8 T cells can protect them from terminal exhaustion. At the epigenetic level, the engineered P14 cells can retain DNA hypomethylation and chromatin accessibility in stemness- and/or effector-associated epigenetic programs during persistent TCR stimulation.

### m) Determine Whether BMP-Signals Counteract TGFβ-Mediated Epigenetic Programming in Exhausted T Cells.

Our data indicate a skewed activation of SMAD1/5-dependent BMP-signaling within partially-exhausted CD8 T cells. To determine whether SMAD1/5-signaling shapes the epigenetic changes to preserve a partially-exhausted state, we can overexpress Smad1 or Smad5 in P14 CD8 T cells using the inducible RV transduction system described above. We can test the effect of the Smad1/5 OE after early activation (day 3) versus effector differentiation of P14 cells in our in-vitro co-culture model. Then, we can track the phenotype and function of OE or control P14 cells during in-vitro prolonged exposure to the tumor antigen in the presence or absence of BMP-2/4. We also can measure DNA methylation levels at the demethylated DMRs that we identified in partially-exhausted CD8 T cells.

To investigate whether augmenting Smad1/5-dependent BMP-signaling in CD8 T cells blocks epigenetic progression toward the full-exhaustion state, we can adoptively transfer transduced or control P14 cells into chronically LCMV-infected or CT2A-gp33 tumor-bearing mice. We can induce Smad1/5 OE in P14 cells 1 day (early OE) or 1-2 weeks (late OE) after the adoptive transfer. We can longitudinally track phenotype and function of transduced P14 cells during chronic infection, or monitor tumor growth in tumor experiments. We can assess the phenotype, function, and epigenetic changes of OE P14 cells, at the chronic stage of the immune response (>35 days post-chronic LCMV infection, >4 weeks after tumor implantation).

Although the data show that the addition of exogenous BMP-2/4 cytokines may not enhance the effector function of CD8 T cells in vitro, it demonstrates that BMP-2/4 can prolong the survival of persistently stimulated P14 cells. Thus, Smad1/5 OE can improve the fitness and survival of P14 CD8 T cells during chronic LCMV infection or cancer. Also, the OE P14 cells can maintain an epigenetic signature and phenotype (Tcf1+ Gzmb^{low} Tim-3− CD39^{low}) similar to that of partially-exhausted T cells.

n) Test Targeting TGFβ1-Signaling and/or Augmenting BMP-Signaling to Enhance Responses to ICB Therapy by Preserving the Pool of Partially-Exhausted T Cells.

The data demonstrate that RepSox treatment—a small molecule inhibitor of Tgfbr1-prevents functional impairment of persistently stimulated P14 cells and reprograms their expression of silenced lymphoid tissue-homing molecule. Therefore, we can test the therapeutic ability of RepSox for maintaining the partially-exhausted state and/or reversing full exhaustion of P14 cells during chronic virus infection or cancer. We can adoptively transfer WT P14 cells into chronically LCMV-infected or CT2A-gp33 tumor-bearing mice. Then, we can treat these mice with RepSox starting 1-2 weeks (early treatment) or 3-4 weeks (late treatment) post-adoptive transfer followed by PD-L1 blockade for 2 weeks. In a complementary set of experiments, we can test the effect of early treatment using BMP-signaling agonists (e.g., BMP-2/4) as monotherapy or combined with RepSox. We can measure phenotypic, functional, and epigenetic changes in P14 cells during each sequential treatment. We also can measure tumor growth and viral titers in the treated mice. We also can test the response to PD-L1 blockade therapy in vivo of genetically edited P14 cells or those expressing cellular factors that prevent full exhaustion. In these experiments, we can treat chronically infected or tumor-bearing, chimeric mice with anti-PD-L1 (>35 d.p.i or 4 wks after tumor-implantation) for 2 weeks. Then, we can analyze proliferation, function, and epigenetic changes of the rejuvenated P14 cells.

Genetic or therapeutic targeting of TGFβ1-signaling in CD8 T cells can synergize with ICB treatment to enhance their proliferation response. Mechanistically, blocking TGFβ1-signaling in persistently stimulated CD8 T cells can prevent their progression toward the full-exhaustion state by maintaining an epigenetic signature of partially-exhausted cells.

### o) Blocking De Novo DNA Methylation Skews the Open Chromatin Landscape of Persistently Stimulated CD8 T Cells Toward the Functional Memory State.

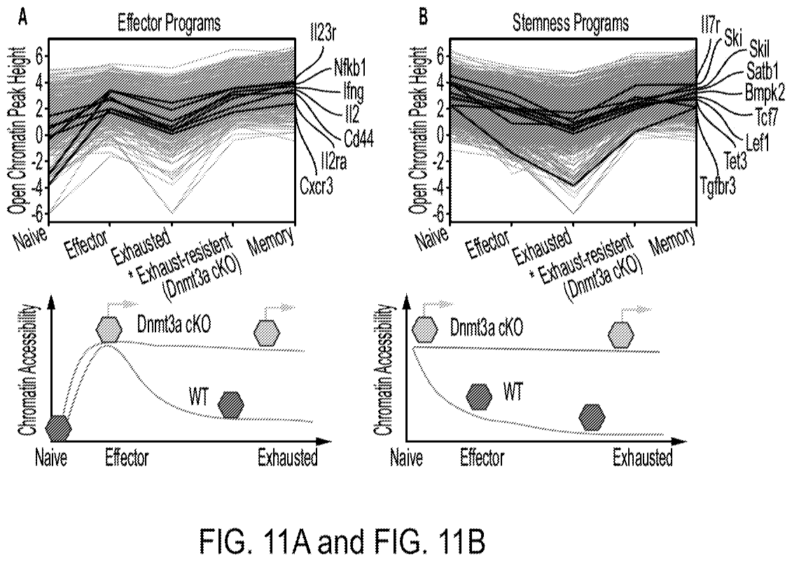

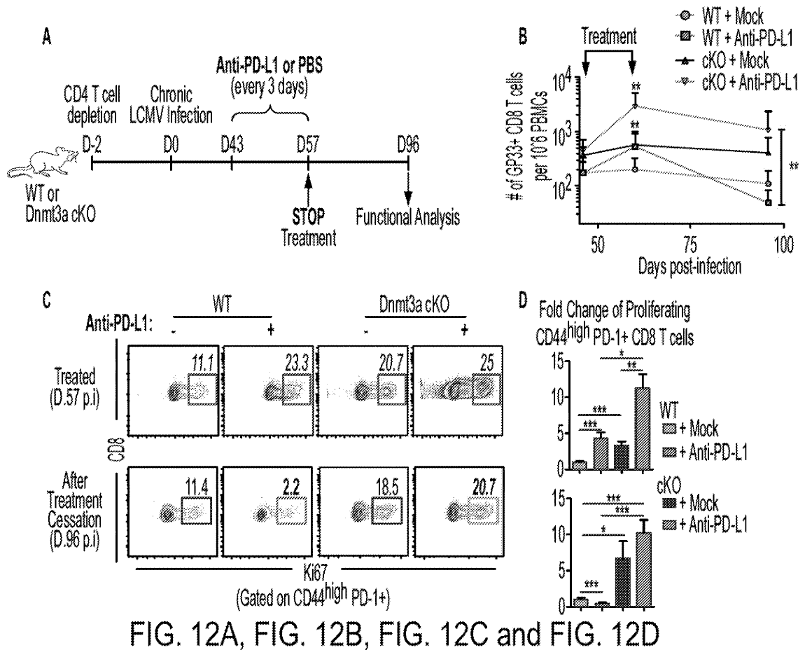

The work demonstrates that de novo DNA methylation regulates the open chromatin landscape in exhausted CD8 T cells. Specifically, we found that CD8 T cells lacking de novo DNA methylation (Dnmt3a cKO) retain high chromatin accessibility at effector function- and stemness-associated genes relative to WT exhausted T cells during chronic LCMV infection (FIG. 11). Many of these genes also are functionally important and similarly accessible in memory CD8 T cells, such as IFNγ, IL2, TCF7, IL7R (FIG. 11). These data indicate that targeting de novo DNA demethylation can re-model exhaustion-specific chromatin accessibility toward the functional memory signature which provides a substantial therapeutic benefit for ICB.

p) Blocking De Novo DNA Methylation in CD8 T Cells Preserves their Potential for Rejuvenation Potential after Cessation of ICB.

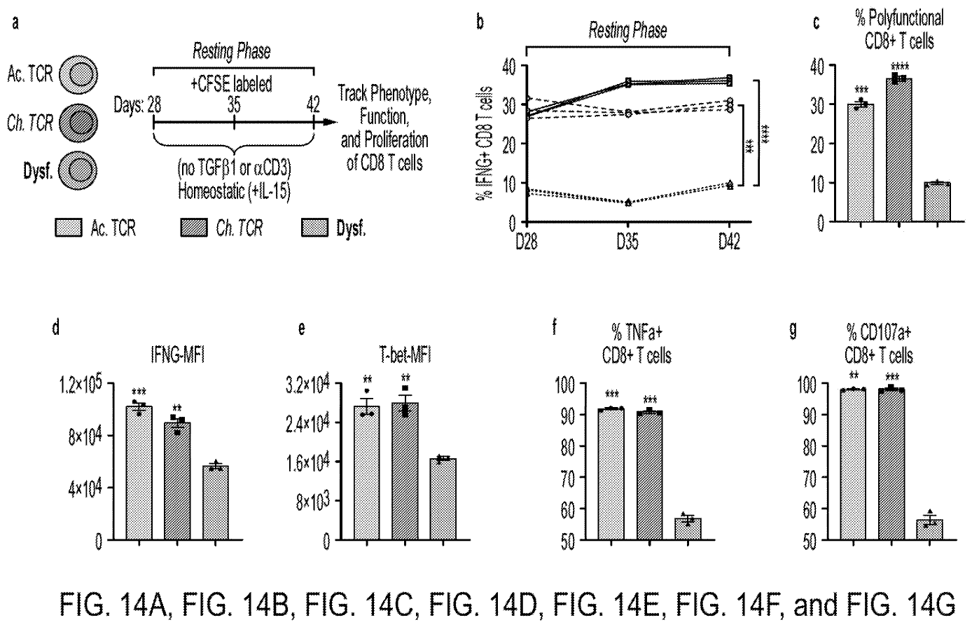

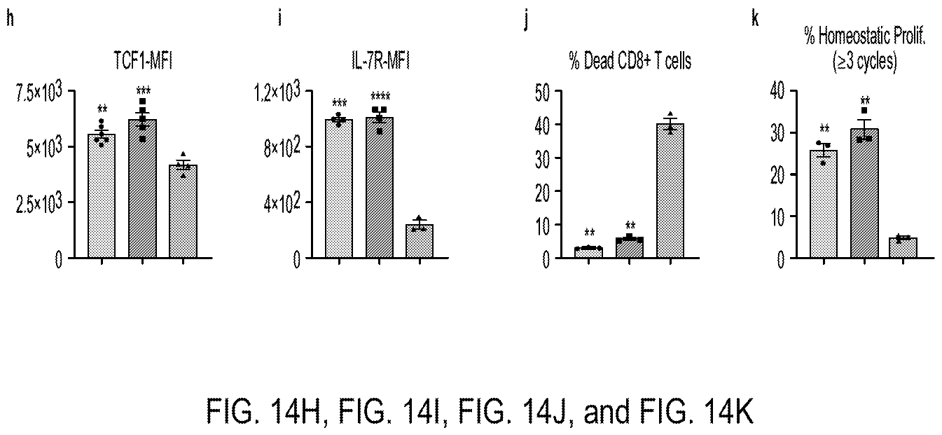

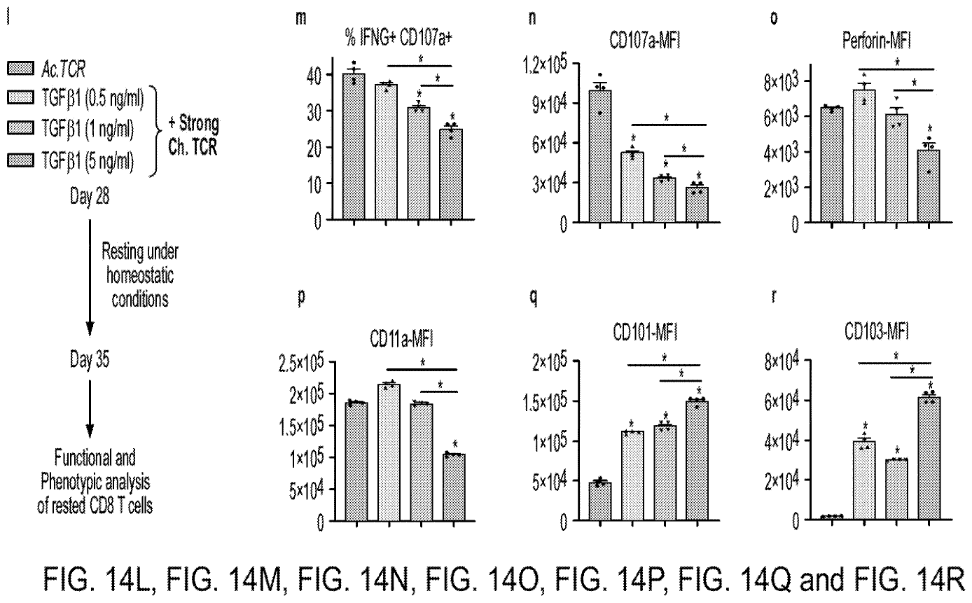

Many patients fail to develop durable protection against tumor relapses after ICB treatment is discontinued. Therefore, it is crucial to develop complementary strategies for generating durable rejuvenation of exhausted CD8 T cells. The work demonstrates that blocking de novo DNA methylation in exhausted CD8 T cells substantially prolongs their rejuvenation potential after discontinuation of ICB treatment. In contrast to WT CD8 T cells, Dnmt3a-deficient cells were maintained at significantly higher numbers during chronic LCMV infection, and exhibited a remarkable proliferative potential for >1 month after cessation of ICB treatment (FIG. 14). These exciting, findings indicate that reactivating epigenetic programs within exhausted CD8 T cells can provide an effective strategy to preserve the proliferative response and memory potential after ICB.

q) Determine Whether Epigenetically Reprogrammed CD8 T Cells Develop Durable Rejuvenation after Stopping ICB Treatment.

The work herein demonstrates that targeting de novo DNA methylation improves the response to ICB, and promotes durable rejuvenation after stopping ICB treatment. We can test the ICB responsiveness of the epigenetically reprogrammed P14 cells using chronic LCMV infection or CT2A-gp33 tumor models. After inducing effective epigenetic reprogramming in the adoptively transferred P14 cells, we can treat chimeric mice with anti-PD-L1 for 2 weeks. We can track changes in phenotype, function, and proliferation response of P14 cells immediately or >1 month after cessation of anti-PD-L1 treatment. We also can test the memory function of the rejuvenated P14 cells by transferring the CFSE-labeled, control or reprogrammed P14 cells after stopping ICB into LCMV-immune mice. Then, we can assess their self-renewal capacity by longitudinal tracking of the CFSE dilution of the transferred P14 cells. We also can test their recall effector function by challenging the LCMV-immune mice with a high dose of the chronic LCMV strain to determine their effector and proliferation response.

The epigenetic reprogramming of exhausted P14 cells can synergize their proliferation response and promote long-lived memory function after ICB treatment.

### Example 2: Rebalancing TGFb1/BMP Signals Revise Effector and Memory Programs in Terminally Dysfunctional CD8+ T Cells

### a) RESULTS

### (1) Post-Effector TGFb1 Signaling Accelerates Severe Dysfunction in Chronically Stimulated Human CD8+ T Cells

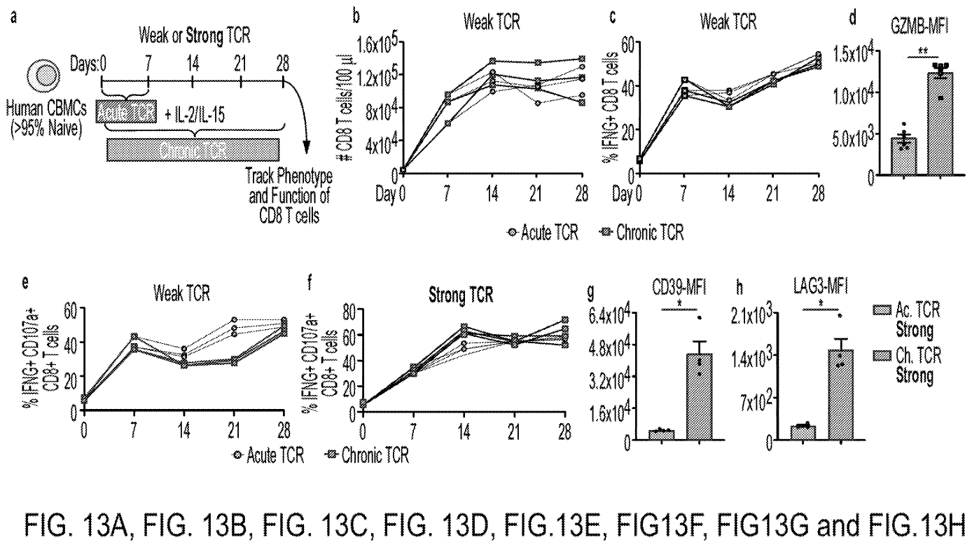

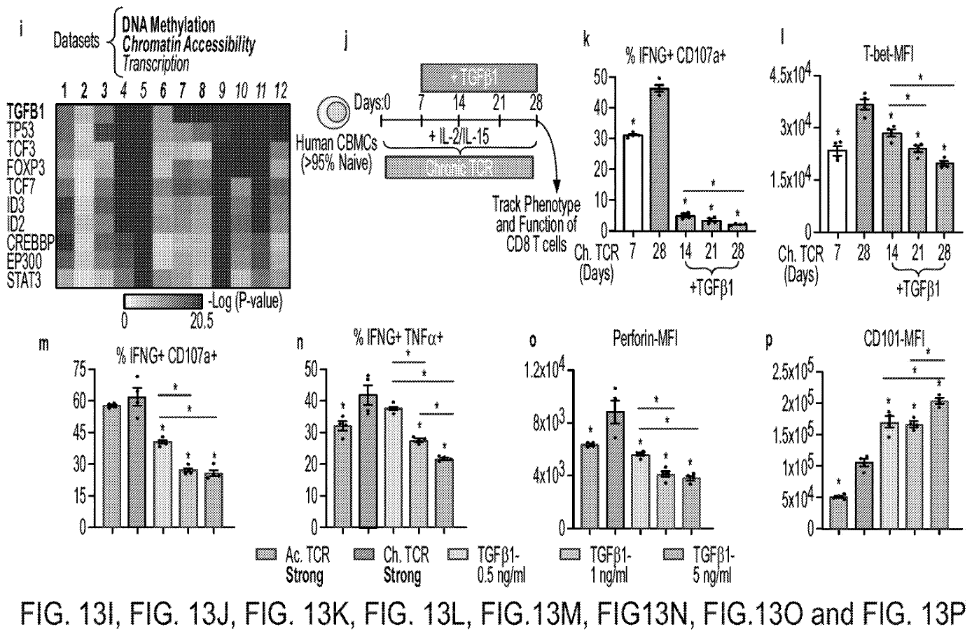

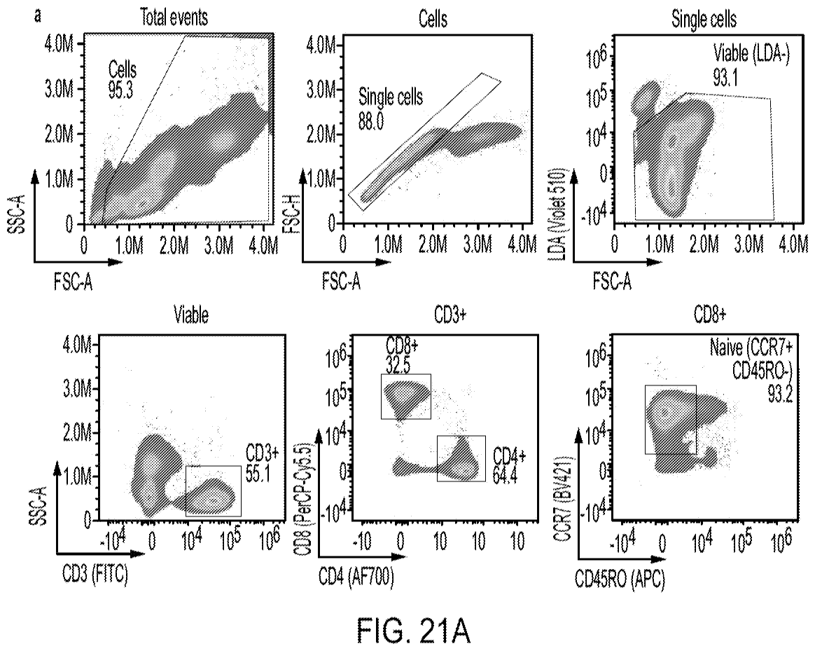

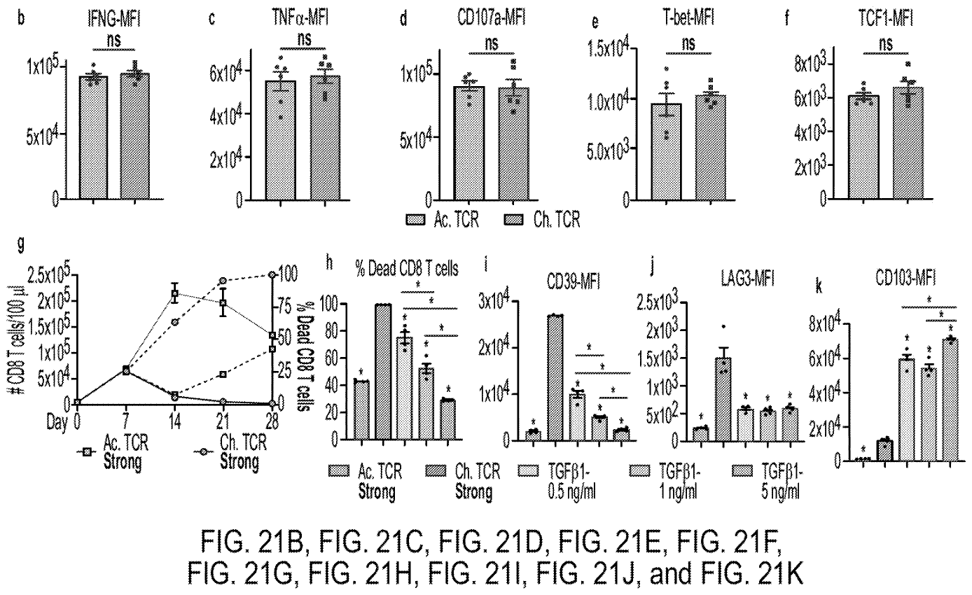

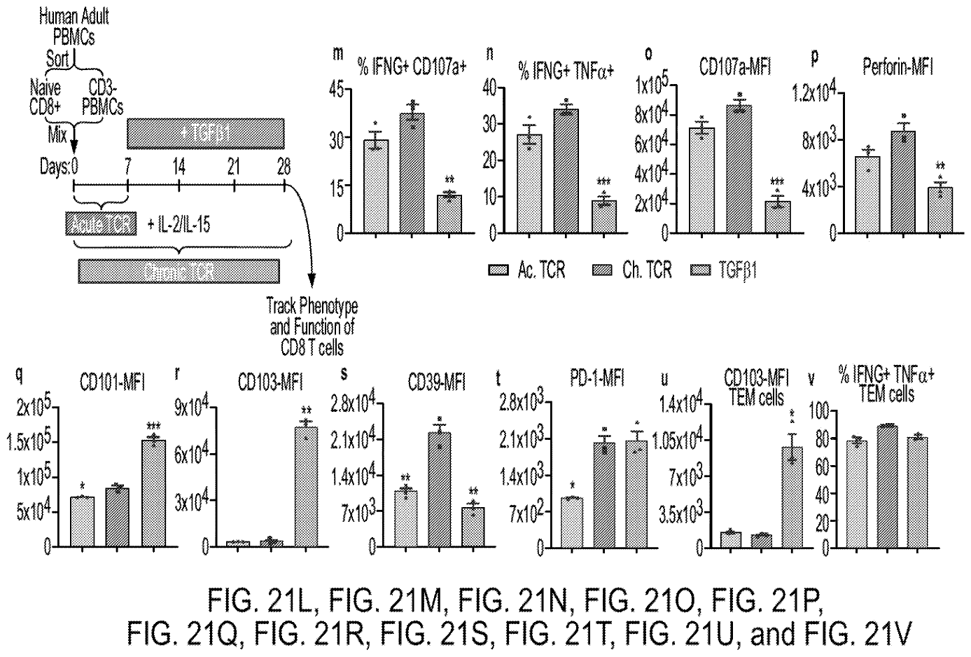

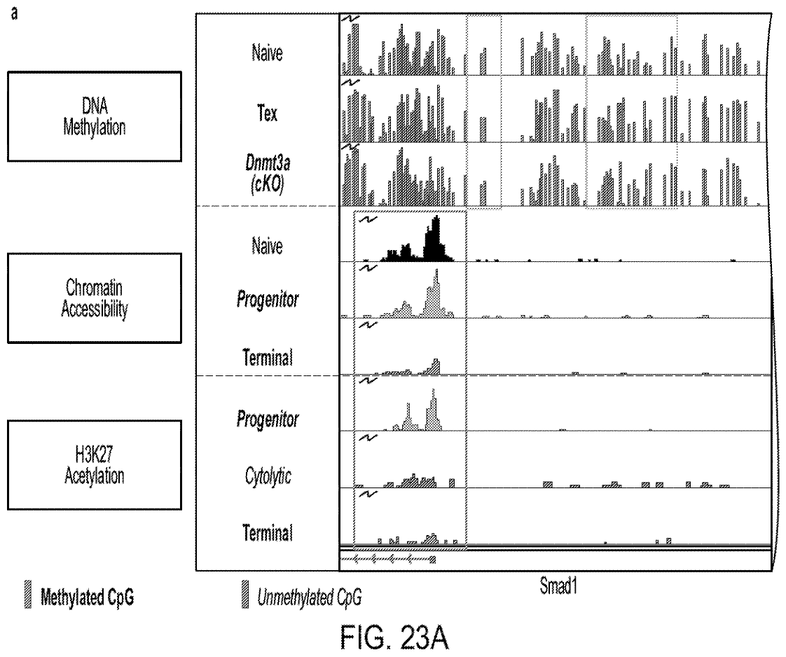

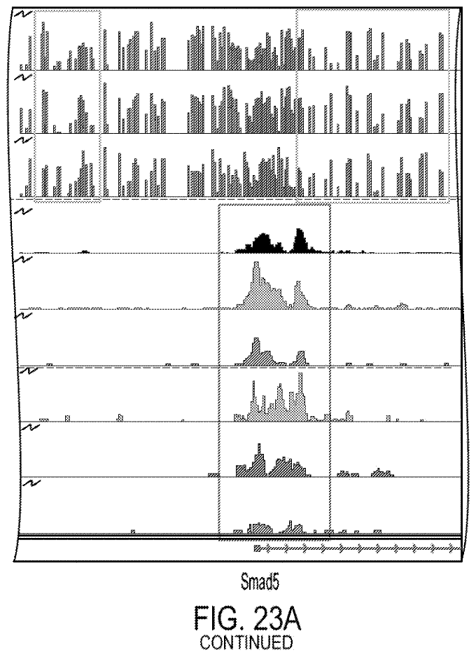

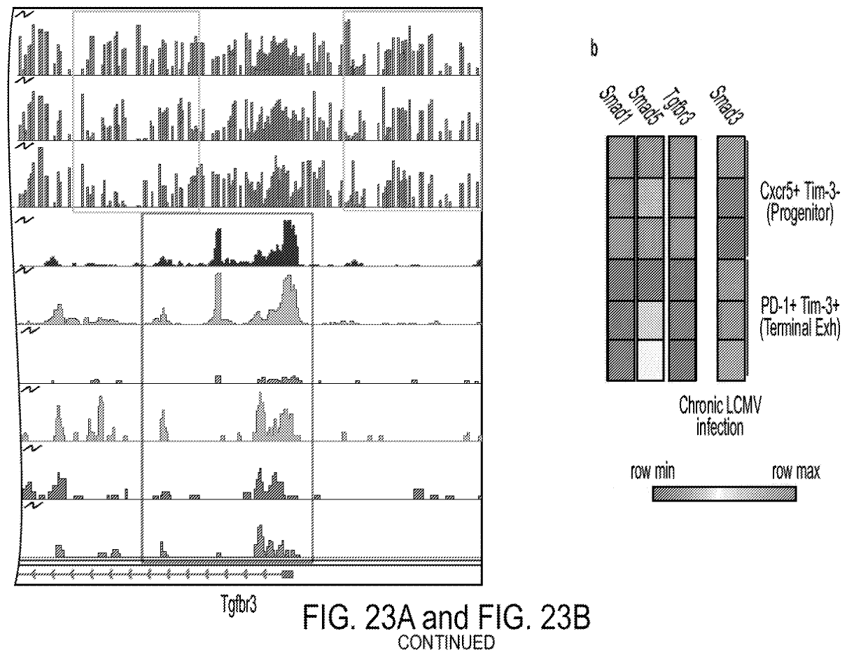

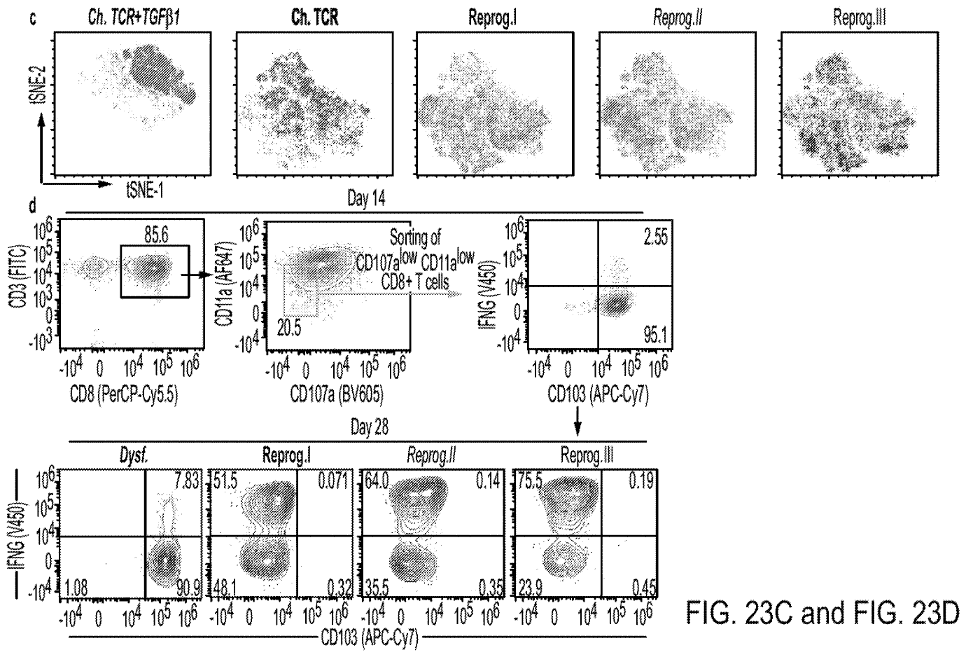

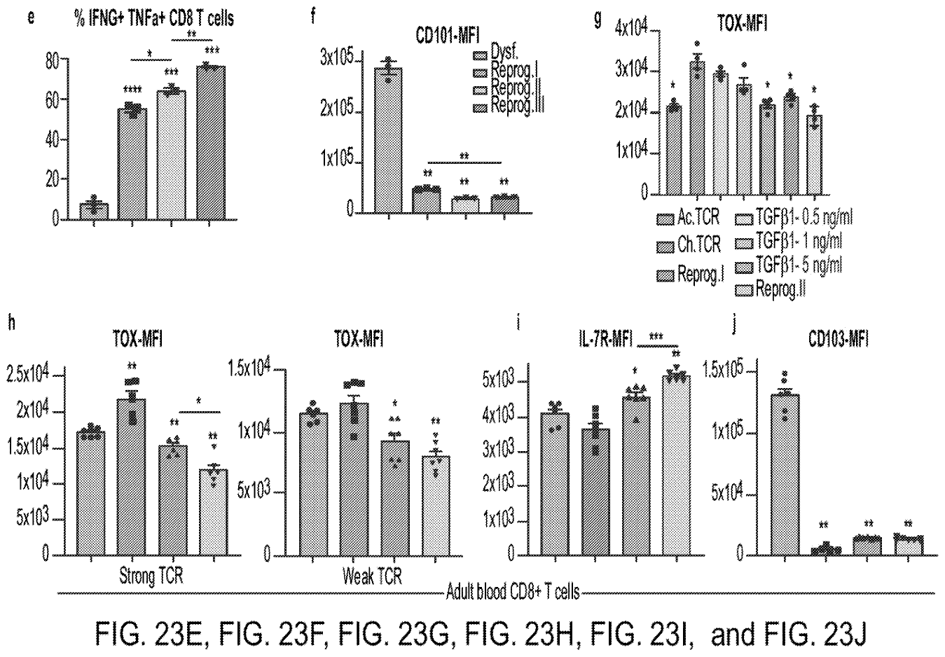

To identify key upstream regulators of human CD8+ T cell dysfunction under controlled conditions, we innovated an in vitro model using long-term cultures of mononuclear cells isolated from neonatal cord blood (CBMCs), in which >95% of T cells are in the naïve state (FIG. 13a, FIG. 21a), or purified naïve adult CD8+ T cells mixed with autologous CD3-PBMCs from healthy blood donors (FIG. 23l). To test whether chronic TCR stimulation can drive a dysfunctional program, we stimulated CBMCs using soluble anti-CD3 plus natural co-stimulation (provided by CD80/CD86 on Fc-expressing CB monocytes) with IL-15 and IL-2 for 1 week (acute stimulation) or >4 weeks (chronic stimulation) (FIG. 13a). We longitudinally assessed the functional capacity of acutely or chronically stimulated CD8+ T cells via their expression of effector molecules (IFNγ, TNFα, GZMB) and the degranulation marker CD107a following PMA/ionomycin stimulation. As expected, acute TCR stimulation induced significant expansion of human CD8+ T cells (˜30-40-fold) that acquired an effector phenotype with elevated cytokine expression (FIG. 13b-e). When subsequently rested in IL-2/-15 for an additional 7 to 28 days, the effector cells maintained polyfunctionality and expressed high levels of memory-related molecules, including the transcription factors T-bet and TCF1 (FIG. 21b-f). Surprisingly, CD8+ T cells chronically stimulated through their TCRs maintained polyfunctionality and high levels of effector and memory molecules (FIG. 13b-d, 21g-j. FIG. 21b-g). To determine the impact of TCR strength on the induction of T cell dysfunction, we modified our human in vitro model to provide prolonged strong TCR stimulation of human CD8+ T cells using plate-bound anti-CD3/CD28 (first week) followed by repeated plate-bound anti-CD3 stimulation (day 7-28) (FIG. 13a). Relative to weak TCR stimulation, persistent strong TCR stimulation induced faster terminal differentiation and activation-induced cell death, coupled with higher expression levels of inhibitory receptors and “exhausted-like” phenotype molecules (FIG. 13f-h, FIG. 21g-j). Yet, similar to the prolonged weak TCR stimulation setting, chronically stimulated CD8+ T cells maintained their polyfunctionality (FIG. 21f, 1m-o). These data indicate that chronic TCR stimulation of human CD8+ T cells is insufficient to develop T cell dysfunction.

Continuous antigenic exposure is thought to be the main driver of T cell dysfunction in tumors or during chronic infections. However, CD8+ T cells chronically stimulated in vivo by self-antigen remain highly functional in autoimmune diseases, indicating an important contribution of concomitant microenvironmental cues in establishing T cell dysfunction. Initially, we sought to identify conserved upstream regulators of dysfunction-associated molecular programs using multi-comparisons Ingenuity Pathway Analysis (IPA) of changes in DNA methylation, chromatin accessibility, and gene expression patterns in mouse and human CD8+ T cell subsets. We compiled 20 datasets for the listed comparisons, including dysfunctional versus functional (memory or dysfunction-resistant Dnmt3a-deficient), terminally dysfunctional versus progenitor or cytolytic CD8+ T cell subsets, or late dysfunctional versus early dysfunctional tumor-specific CD8+ T cells. Our integrative analysis revealed TGFβ1 as the most significant upstream regulator of epigenetic and transcriptional changes in dysfunctional mouse and human CD8+ T cells (FIG. 13i).

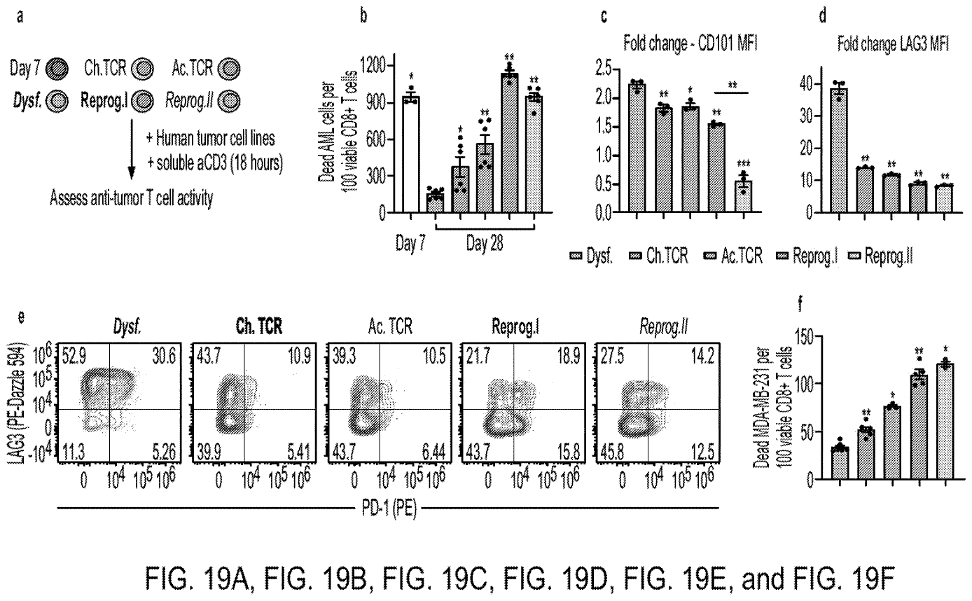

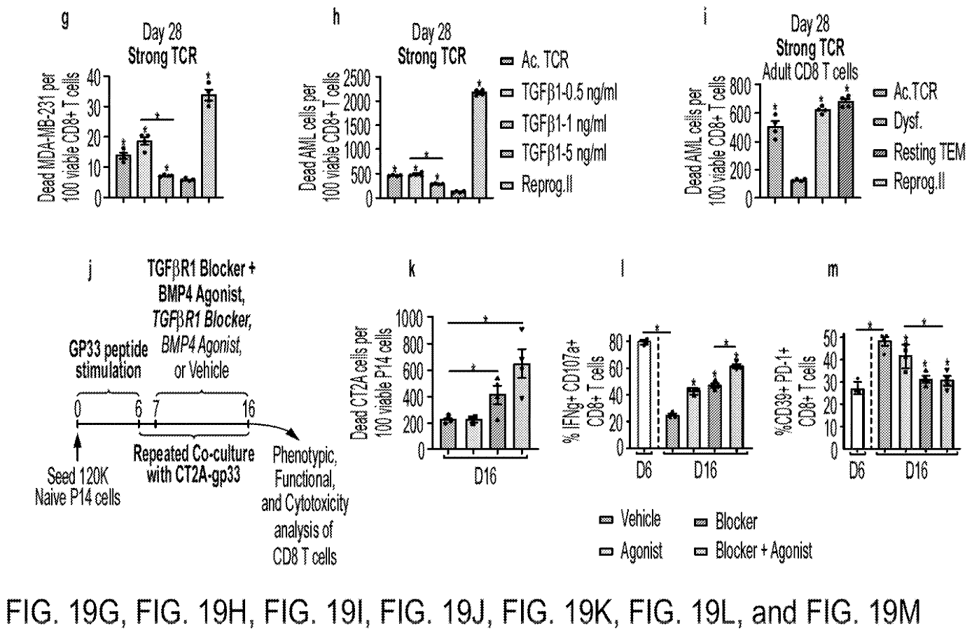

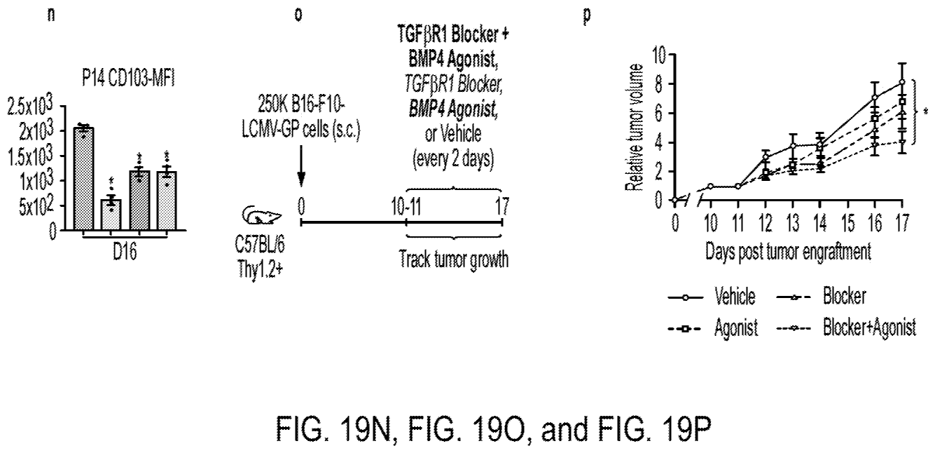

TGFβ1 signaling has been shown to directly suppress cytotoxic function and proliferation during effector CD8+ T cell differentiation and modulate CD4+ T cell differentiation. However, the impact of TGFβ1 signaling on the molecular programs that drive effector T cell progression towards dysfunction remains largely unknown. As shown in FIG. 13j, prolonged TGFβ1 signals, started after effector differentiation, significantly reduced effector functions in chronically stimulated T cells, with these effects becoming more pronounced after 2-3 weeks of exposure. Progressive dysfunction was indicated by decreased frequency of IFNγ-secreting or polyfunctional (IFNγ+ TNFα+) CD8+ T cells (FIG. 13k-n). Moreover, the remaining IFNγ-secreting CD8+ T cells under chronic TGFβ1 exposure exhibited a limited functional capacity indicated by significantly lower expression levels of T-bet and CD107a proteins (FIG. 13l). Importantly, chronic TGFβ1 signals supported the survival of chronically stimulated CD8+ T cells when they experienced strong TCR stimulation. This enhanced survival is likely mediated by attenuating TCR overstimulation (reflected by reduced expression levels of CD39-a marker of high-affinity TCR stimulation in mouse and human CD8+ T cells); thereby suppressing activation-induced cell death (FIG. 21h-j). Notwithstanding, the surviving CD8+ T cells eventually progressed to a dysfunctional state (FIG. 19m-p, FIG. 21k).

Furthermore, we tested the impact of chronic TCR and TGFβ1 signaling using adult naïve CD8+ T cells isolated from healthy blood donors (FIG. 21l). Similar to results obtained with CBMCs, chronically TCR stimulated, adult naïve CD8+ T cells maintained their polyfunctionality. However, post-effector exposure to chronic TGFβ1 signals induced functional impairment and increased expression of dysfunction-associated markers in chronically stimulated adult CD8+ T cells (FIG. 19m-t). We also observed a lower proliferative capacity within the chronically stimulated adult, relative to neonatal, CD8+ T cells. Importantly, chronic TGFβ1 plus TCR stimulation of adult FACS-purified, polyclonal effector memory (CD45RO+ CCR7−) CD8+ T cells did not impact their remarkable polyfunctionality, despite significant upregulation of CD103 (a downstream target of TGFβ1 signaling) (FIG. 21u,v). These data are consistent with the reported resistance of differentiated, primary memory T cells to functional exhaustion following a new chronic infection in mice (though such resistance is reduced with repeated infections). Taken together, we conclude that chronic TCR stimulation (strong or weak), despite being necessary, is insufficient to drive human T cell dysfunction. Instead, chronic exposure to TGFβ1, beginning at the post-effector stage, promotes a transition to profound dysfunctionality in chronically stimulated human CD8+ T cells.

### (2) Prolonged TGFB1 Signaling Establishes a Stable Dysfunctional Program in Chronically Stimulated CD8+ T Cells

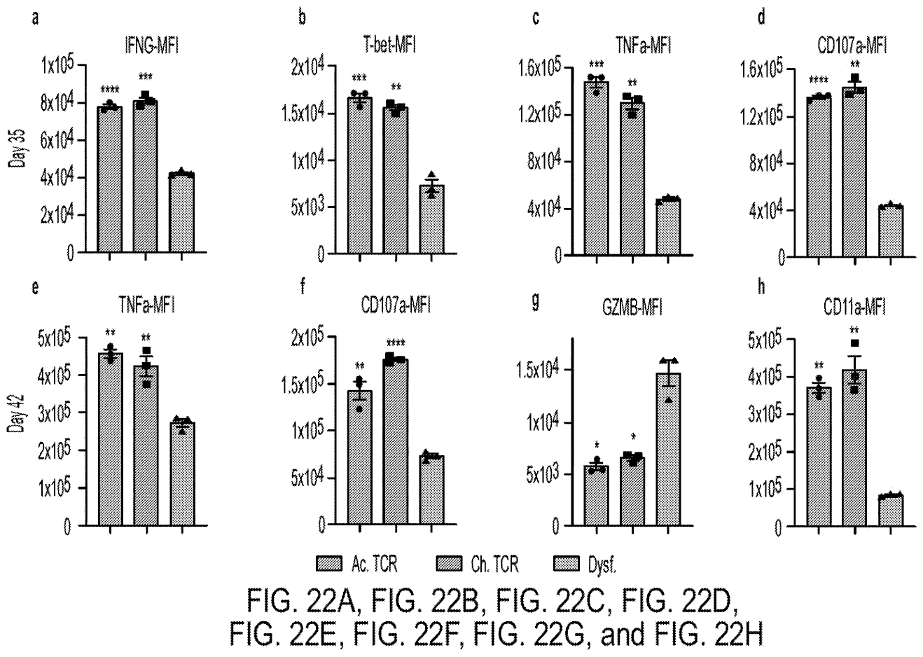

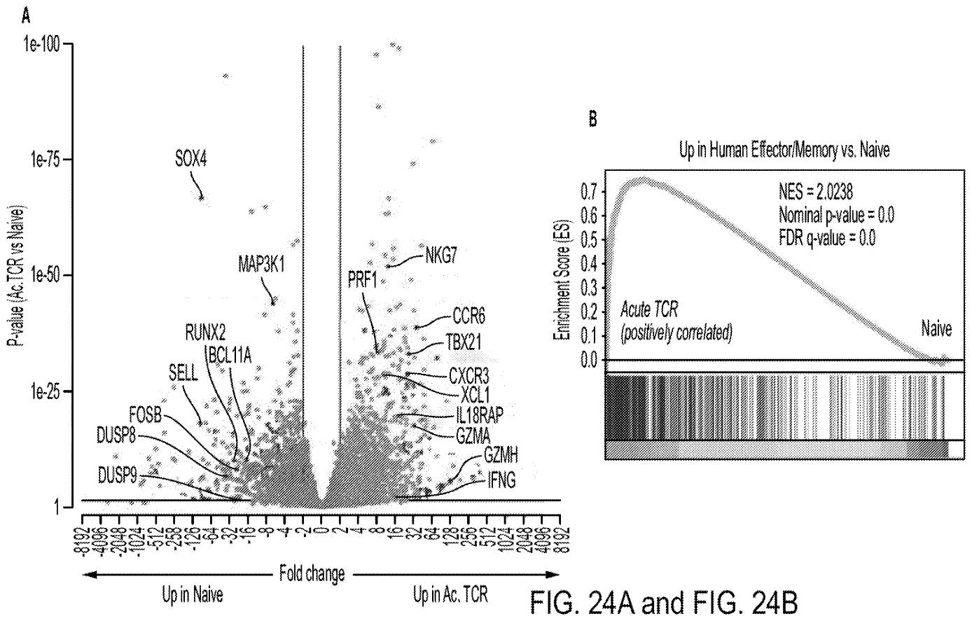

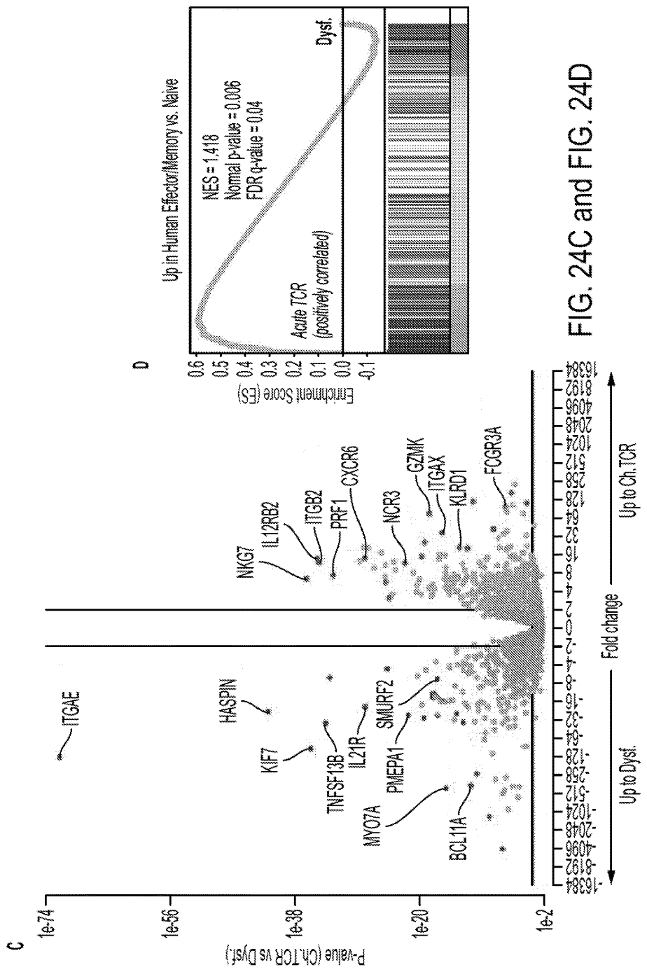

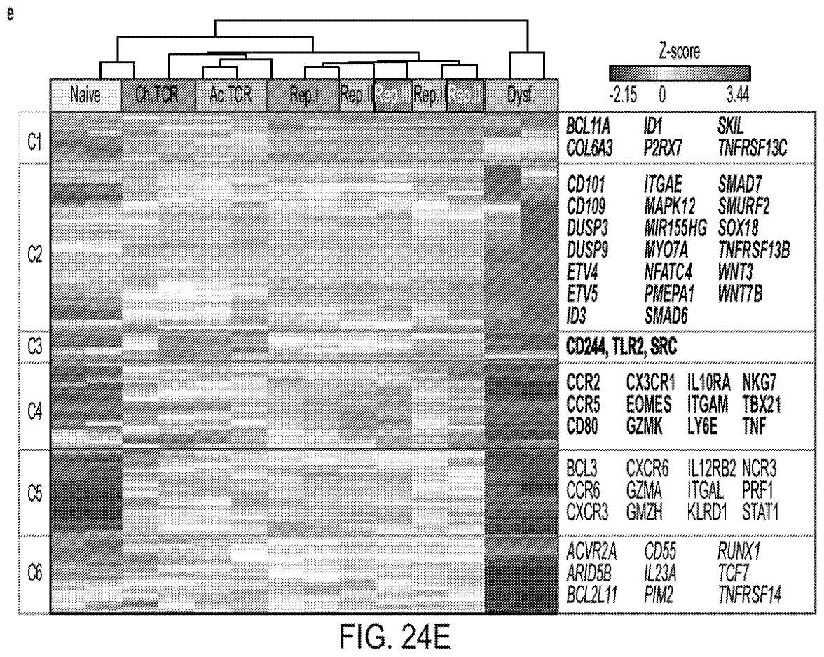

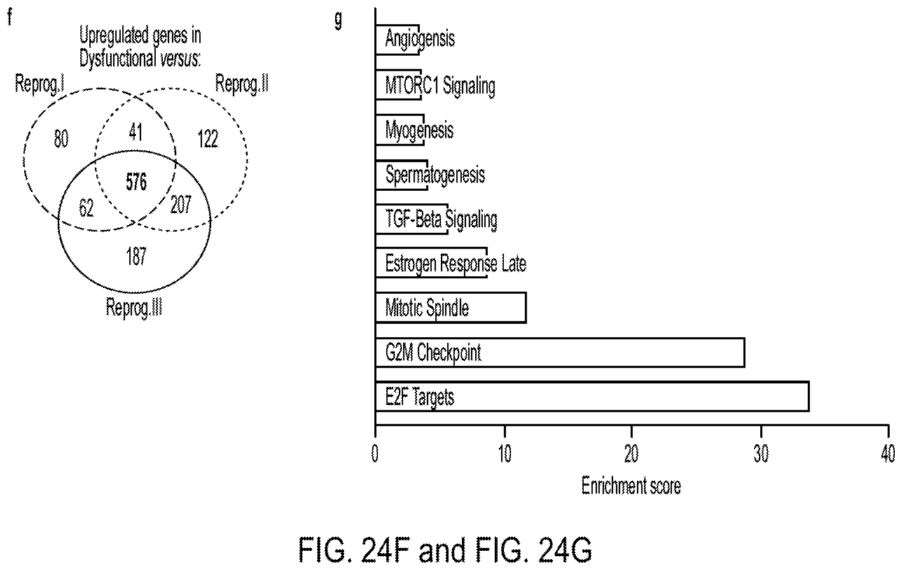

In contrast to memory or early dysfunctional (i.e., progenitor) CD8+ T cells, terminally dysfunctional CD8+ T cells lose their ability to restore effector functions, undergo homeostatic proliferation, and survive after TCR stimulation is withdrawn¹. To determine the fate commitment of dysfunctional CD8+ T cells in our culture system (Chronic TCR plus TGFβ1: hereafter referred to as “Dysf.”), we terminated chronic stimulation and rested the cells under homeostatic conditions for an additional 7-14 days, beginning on day 28 (FIG. 14a). Human CD8+ T cells that were propagated under acute or chronic TCR stimulation settings, retained polyfunctionality with memory-like features during the resting phase. In contrast, neither effector cytokine production nor expression of memory markers, such as T-bet, IFNγ, TNFα, CD107a, IL-7R, and TCF1, was restored in recovered Dysf. CD8+ T cells (FIG. 14b-i, FIG. 24). Rested Dysf. CD8+ T cells also maintained significantly higher levels of GZMB (FIG. 22g) and declined cell numbers, features often associated with terminal dysfunction.

To determine whether the observed decline in dysfunctional T cell quantity arises from impaired homeostasis or reduced survival, we measured self-renewal capacity and cell death of resting CD8+ T cells by CFSE labeling of acutely or chronically stimulated T cells on day 28 (FIG. 14a). The Dysf. CD8+ T cells displayed impaired proliferative capacity coupled with enhanced cell death, properties that were not reversed by resting over 2 weeks (FIG. 14j-k). Similarly, Dysf. human CD8+ T cells rested from strong TCR stimulation continued to show impaired polyfunctionality and higher expression of CD101 and CD103 (FIG. 14l-r), demonstrating a stable dysfunctional state after the removal of chronic TCR and TGFβ1 signaling. Such inflexible commitment to terminal dysfunction in Dysf. CD8+ T cells is consistent with the limited functional recovery of exhausted virus-specific CD8+ T cells after clearance of chronic viral infections in humans and mice. In contrast, CAR T cells were recently reported to attain reversible dysfunction during in vitro chronic antigenic stimulation. Collectively, these findings indicate that post-effector, chronic TGFβ1 exposure enforces cell-intrinsic programs for stable, terminal dysfunction in CD8+ T cells experiencing persistent TCR stimulation.

### (3) BMP4 Agonist Treatment Reverses Exhaustion Features of Human CD8+ T Cells Under Chronic Strong TCR-Stimulation

TGFβ1 is a highly conserved cytokine inducing multiple biological functions and pathways that are orchestrated through interactions of SMADs or non-SMAD proteins. While SMAD2/3-dependent events represent the main canonical pathway downstream TGFβ 1 cytokine, other non-canonical pathways can also be activated in a context-dependent manner. These pleiotropic effects prompted us to investigate whether TGFβ signaling is differentially regulated among dysfunctional CD8+ T cell subsets. Importantly, we found that stem-like progenitor CD8+ T cells retain higher expression levels of Smad1/5 and Tgfbr3, coupled with more accessible chromatin and permissive epigenetic programs at these loci (DNA hypomethylation and high H3K27acetylation marks) during chronic virus infection (FIG. 23a,b). Conversely, terminally dysfunctional CD8+ T cells acquire unique DNA methylation changes, coupled with reduced chromatin accessibility, loss of permissive H3K27 acetylation marks, and reduced expression of these key signaling molecules, while maintaining high expression levels of Smad2/3 (FIG. 23a,b). These data suggest that Smad1/5-dependent signaling is preferred in the progenitors of dysfunctional T cells, preserving partial effector function and stemness during chronic stimulation.

The SMAD1/5-dependent pathway is selectively activated by a group of ligands, including bone morphogenetic protein (BMP2/4) cytokines, which also are members of the TGFβ super-family. To determine whether activating the SMAD1/5-dependent pathway attenuates a progression of chronically stimulated T cells to dysfunction, we augmented SMAD1/5-signaling using SB4 (2-[[(4-Bromophenyl)methyl]thio]benzoxazole), as shown in the formula:

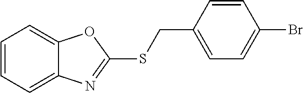 [SMILES: Brc1ccc(CSc2nc3ccccc3o2)cc1]

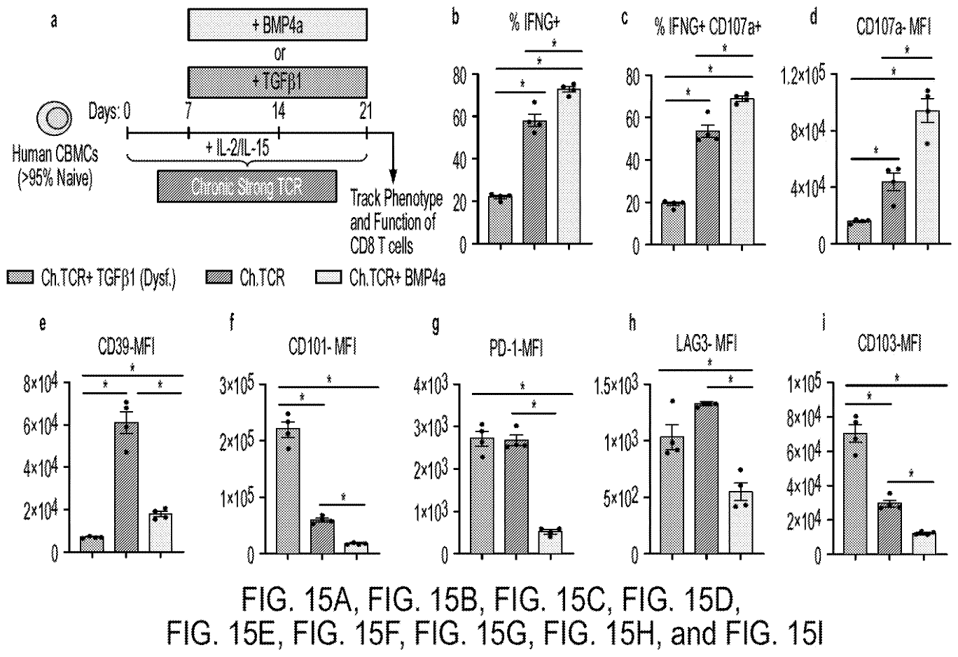

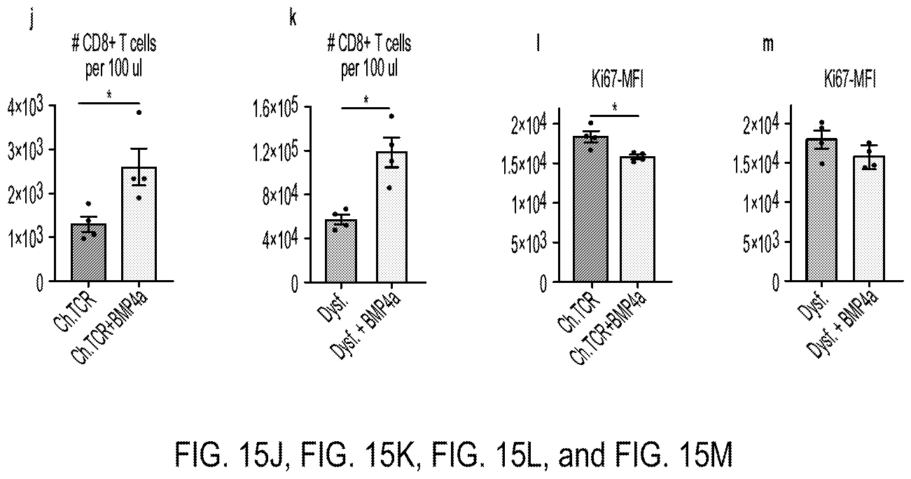

SB4 is a selective small molecule BMP4 agonist (BMP4a) that stabilizes SMAD1/5 phosphorylation—in human CD8+ T cells under chronic strong TCR stimulation (FIG. 15a). Indeed, we found that BMP4 agonist treatment significantly increased polyfunctionality (FIG. 15b-d), while reducing the expression of exhaustion-associated molecules, such as PD-1, LAG3. CD39, CD103, and CD101 (FIG. 15e-i). Importantly. BMP4a treatment rescued significantly higher numbers of persistently stimulated CD8+ T cells without increasing the proliferation activity (FIG. 15j-m). Taken together, these findings reveal a novel role for BMP4 signals in reversing exhaustion features and enhancing the survival of chronically stimulated CD8+ T cells.

### (4) Rebalancing of TGFβ1 and BMP Signaling Reverses Terminal T Cell Dysfunction

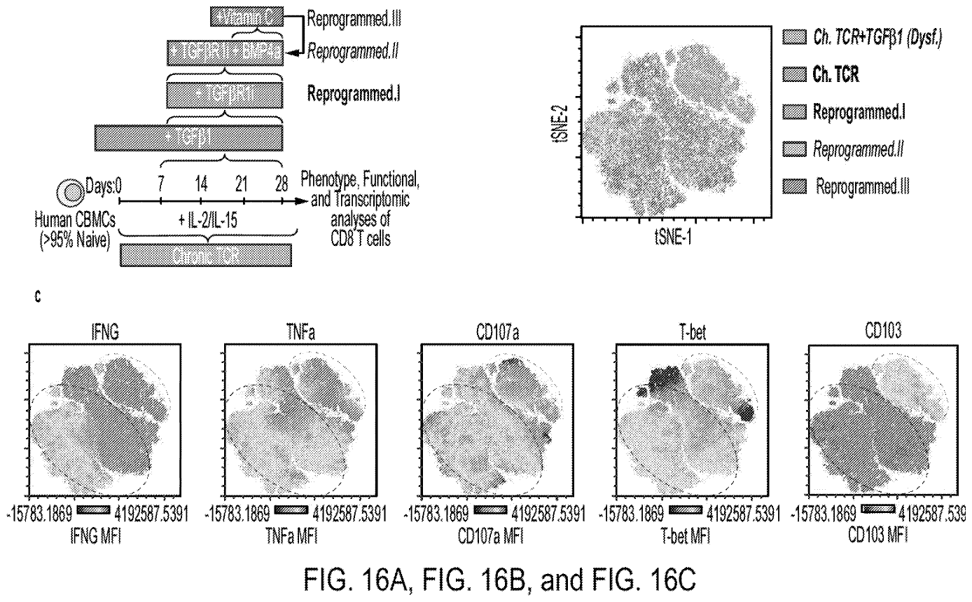

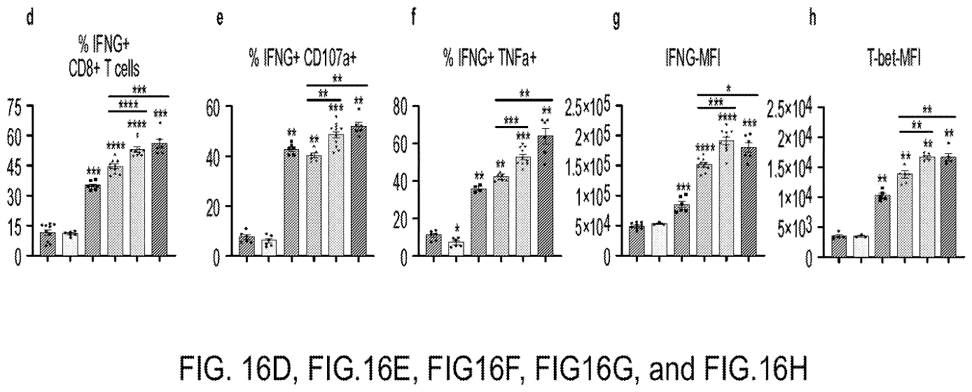

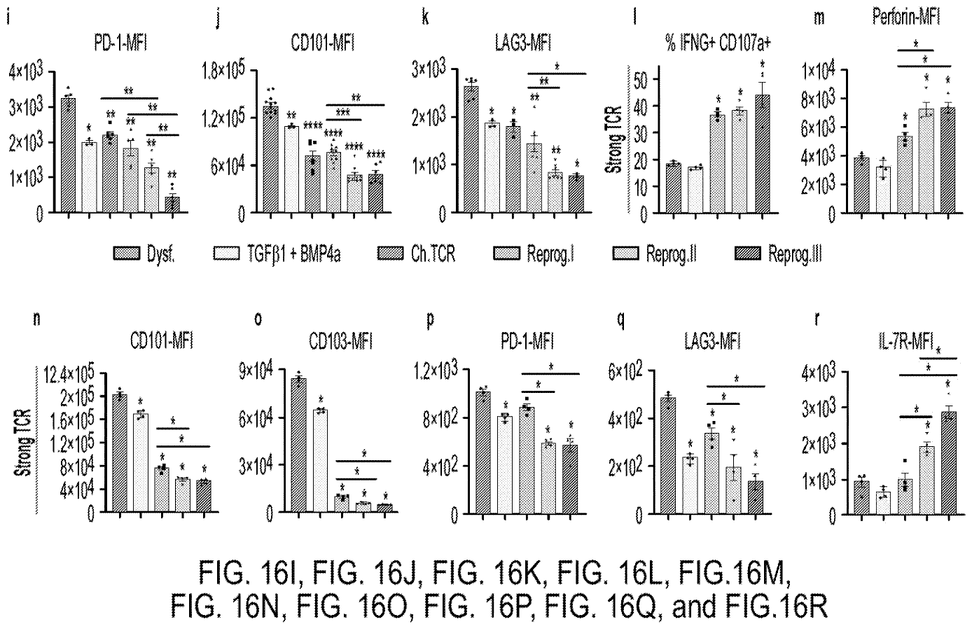

Targeting of TGFβ1 release or receptor signaling has shown therapeutic promise in certain preclinical models of chronic viral infections or tumors. In these systems, enhanced T cell responses were mediated by a modulation of immunosuppressive regulatory CD4+ T cells or myeloid cells, and in some cases by promoting T cell priming or infiltration. Our data support a critical role for TGFβ1 signaling in CD8+ T cell fate commitment to terminal dysfunction. To determine whether targeting TGFβ1 in T cells can modulate their commitment to dysfunction, we blocked TGFβ31 signaling using RepSox—a potent selective inhibitor of the TGFβ type 1 receptor (ALK5) starting in the third or fourth week of chronic stimulation (FIG. 16a). We found that RepSox treatment restored effector molecules (e.g., IFNγ, TNFα, CD107a, T-bet) in Dysf. CD8+ T cells (FIG. 16b-h). In addition, surface markers associated with terminal dysfunction, including CD101 and inhibitory receptors PD-1 and LAG3, were downregulated after RepSox treatment (FIG. 16i-k), confirming a central role for TGFβ1 signaling in driving terminal exhaustion. We then asked whether boosting BMP4 signals alone can reverse TGFβ1-driven exhaustion. We found that BMP4a monotherapy (in the absence of TGFβ R1 blocker) significantly reduced the expression levels of dysfunction-related molecules, such as PD-1, LAG3, CD101, and CD103 (FIG. 16i-k, 16n-p). However, it was not sufficient to restore the effector function of Dysf. CD8+ T cells (FIG. 16d-h. To further enhance the reversal of terminal dysfunction in chronically stimulated CD8+ T cells, we rebalanced TGFβ and BMP4 signals by combining TGFβ R1 blockade with BMP4 agonist treatment. Importantly, BMP4a therapy significantly boosted the recovery of polyfunctionality and cytotoxic and memory programs (e.g., IFNγ. TNFα, CD107a, T-bet, Perforin, IL-7R), while further reducing the expression levels of terminal dysfunction molecules in RepSox-treated Dysf. CD8+ T cells (FIG. 16d-r). To further enhance the recovery potential of Dysf. CD8+ T cells, we added physiological levels of vitamin C (day 21-28), which acts as an important co-factor for active. DNA. Treatment of Dysf. CD8+ T cells with vitamin C late in the culturing regimen augmented their polyfunctional recovery and survival (FIG. 16), suggesting an epigenetic link to this process. Our results suggest a significant role for SMAD1/5-dependent B3MP signals in restoring the functionality and memory programs within chronically stimulated CD8+ T cells.

To determine whether BMP-dependent recovery of Dysf. CD8+ T cells is due to reprogramming events, rather than selective enrichment of a less dysfunctional subset, we isolated the most dysfunctional subset (CD107a^{low} CD11a^{low}) from chronically stimulated human CD8+ T cells on day 14 (TCR plus TGFβ1) and confirmed their limited capacity to produce IFNγ upon in vitro stimulation (FIG. 23d). After treatment with RepSox (2-weeks), the most dysfunctional CD8+ T cells significantly recovered effector cytokine production that was further enhanced by concomitant BMP4 agonist and late vitamin C treatment (FIG. 23d,e). Furthermore, Dysf. CD8+ T cells maintained high expression levels of CD103 and CD101, while treated cells downregulated these terminal dysfunction-related markers (FIG. 23d,f). Similarly, these reprogramming mechanisms restored polyfunctionality, promoted the expression of effector and memory-related molecules (Perform, IL-7R), and reduced CD101 and CD103 expression in chronically stimulated CD8+ T cells under strong TCR and TGFβ1 signals (FIG. 16m-r, FIG. 23h-j). These findings demonstrate that modulating TGFβ1/BMP signals can actively reprogram terminally dysfunctional CD8+ T cells, restoring their functionality.

(5) Transcriptional Reprogramming of Dysfunctional CD8+ T Cells into a Memory-Like State

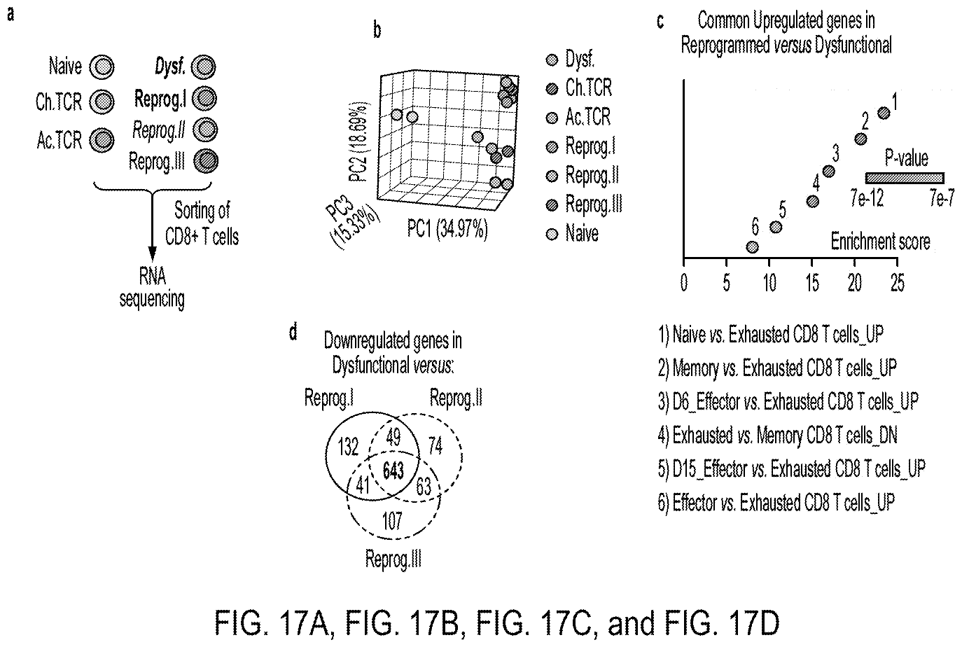

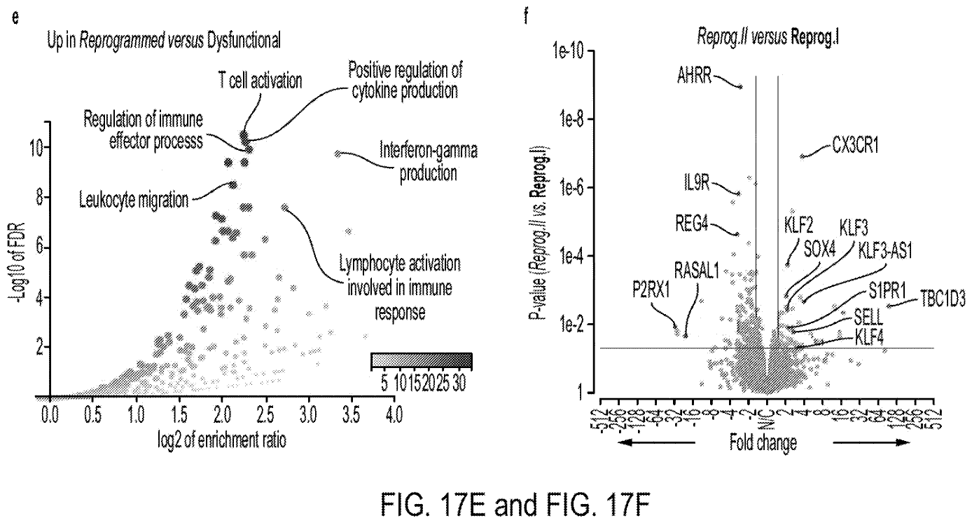

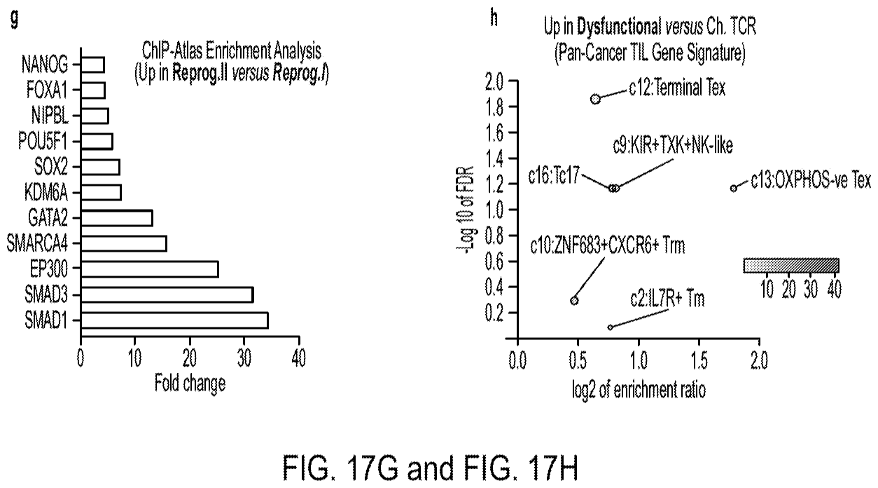

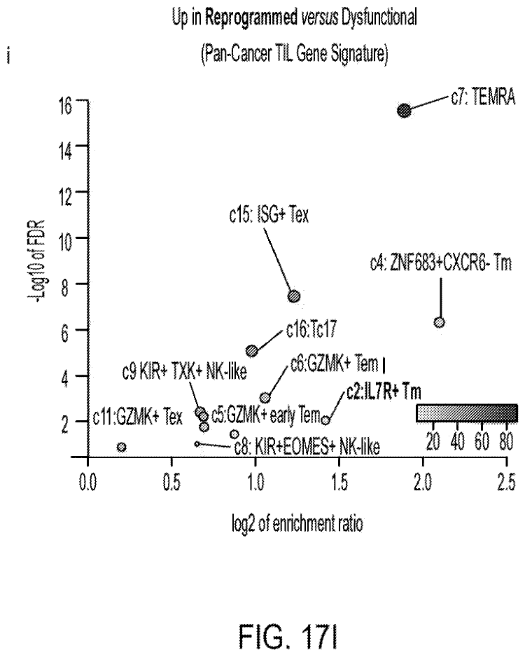

To define the molecular underpinnings of terminal dysfunction and TGFβ1/BMP-mediated functional switches, we performed RNA-sequencing analyses on human CD8+ T cells under a variety of acute or chronic stimulation settings (FIG. 17a). After acute TCR stimulation, we found that human CD8+ T cells underwent major transcriptional changes that significantly overlap with gene expression programs acquired in antigen-specific effector and memory CD8+ T cells after yellow fever vaccination (FIG. 24a,b). Examples include high expression of effector programs, such as IFNG, TNF, GZMA, PRF1, GNLY, NKG7, TBX21, and PRDM1, as well as naïve/memory-related genes, such as TCF7, CCR7, IL7R (FIG. 24a). In keeping with functional assays, CD8+ T cells differentiated under chronic TCR stimulation alone maintained effector function programs and a similar transcriptional profile to that of acutely stimulated CD8+ T cells (FIG. 17b, , FIG. 24c). In contrast, Dysf. CD8+ T cells acquired a distinct transcriptional profile (FIG. 17b) characterized by a significant loss of: (i) effector programs (e.g., Granzymes, PRF1, NKG7, ITGAL, TNFRSF4, TNFRSF18), (ii) key chemokine and cytokine receptors (e.g., CXCR3, CXCR6, CCR2, CCR8, IL12RB2), and (iii) memory-related transcription factors (TFs), such as TBX21 and TCF7 (FIG. 24c-e). Additionally, the dysfunctional cells upregulated multiple negative regulators of TCR signaling (e.g., DUSP3, DUSP4, DUSP9), a specific set of integrins (e.g., ITGAE, ITGB3, ITGB4), and genes linked to human T cell dysfunction (e.g., CD101, ID1, ID3). Importantly, we observed an upregulation of several genes in Dysf. T cells that attenuate TGFβ signaling, such as SMURF2, PMEPA1, SKIL, SMAD6, and SMAD7, implying engagement of negative feedback mechanisms during prolonged TGFβ1 exposure (FIG. 24e). Overall, these results indicate that chronic TGFβ1 signaling modulates transcriptional programming within chronically stimulated CD8+ T cells, propelling them toward terminal dysfunction.

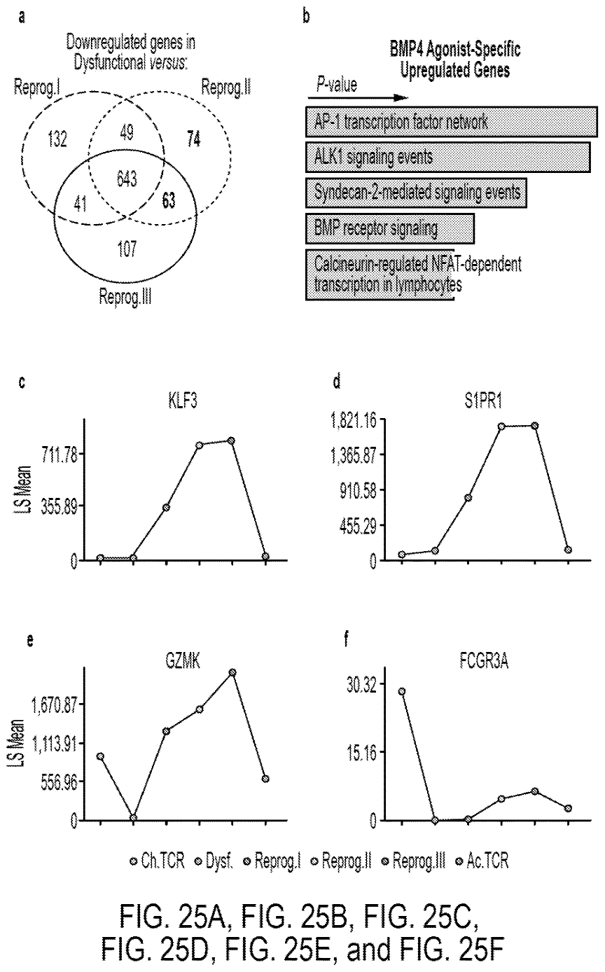

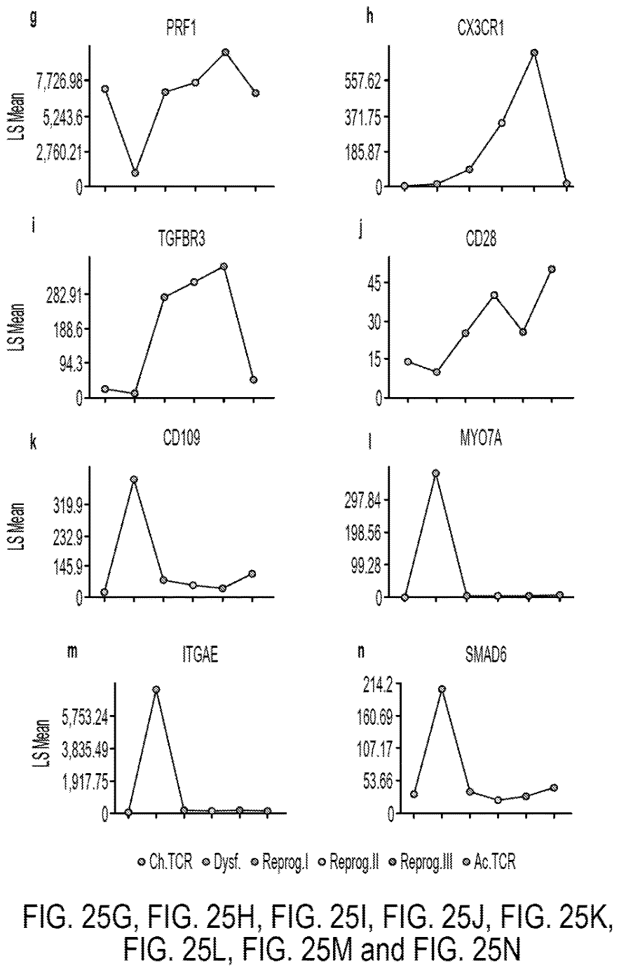

We next examined the impact of rebalancing TGFβ1/BMP signals in Dysf. CD8+ T cells, with and without inclusion of vitamin C (FIG. 17a). We found that blockade of TGFBR1 alone restored substantial expression levels of many effector function-related genes (e.g., TBX21, PRF1, IFNG, GZMK, GZMH, CX3CR1, CXCR3, NKG7, GNLY), in addition to some memory-related programs, such as TCF7, IL7R, SELL, SATB1, KLF2, NT5E, and ITGAL (FIG. 24e). The recovery of effector and memory programs was further potentiated by the BMP4 agonist, as well as with late vitamin C treatments (FIG. 24e). Enrichment analyses of the recovered genes in Reprogrammed CD8+ T cells showed strong enrichment in effector and memory gene expression programs that are lost in bona fide exhausted CD8+ T cells (FIG. 17c) Furthermore, the commonly recovered genes showed significant skewing in biological processes and gene sets of functional T cells, including interferon-gamma production, leukocyte activation and migration, and positive regulation of cytokine production (FIG. 24). In contrast, the commonly upregulated genes in Dysf. versus Reprogrammed CD8+ T cells were enriched in E2F targets, cell cycle G2M checkpoint, and TGFβ signaling (FIG. 24f,g). Importantly, the BMP4 agonist treatment re-activated expression of multiple key genes regulating T cell co-stimulation, chemotaxis, and sternness, such as CD28, FOSB, FCGR3A, CXCL8, and POU2F2, in addition to BMP receptor/co-receptor genes (e.g., RGMB, ACVR1) (FIG. 24e). In addition, it boosted the recovery of multiple TFs whose activities were recently shown to be significantly enriched in human naïve and memory CD8+ T cells, such as KLF3, as well as enhancing the expression of multiple effector/memory programs (FIG. 17f, FIG. 25c-j). In this regard, the set of genes upregulated specifically by the BMP4 agonist was significantly enriched in TCR signaling-related pathways, including the AP-1 TF network and calcineurin-regulated NFAT-dependent transcription (FIG. 25a,b).

The remarkable enhanced survival and recovery of memory programs in CD8+ T cells during BMP4 agonist therapy prompted us to examine whether stem cell-associated regulators are enriched in BMP-driven reprogramming. We performed ChIP-atlas enrichment analysis for transcriptional regulators with enriched binding within 5 Kb+/− TSS of genes that are uniquely upregulated in reprogrammed CD8+ T cells under BMP4 agonist treatment (i.e., Reprog.II versus Reprog.I) using embryonic stem cells and pluripotent stem cells databases. This analysis revealed enrichment of multiple binding elements for SMAD1/3, EP300, SMARCA4, POU5F1, and NANOG, implying important roles in regulating BMP-driven transcriptional reprogramming (FIG. 17g). Conversely, augmenting BMP4 signaling in reprogrammed CD8+ T cells significantly downregulated several genes linked to purinergic signaling at the immune synapse, attenuation of TCR signaling or anti-tumor T cell immunity, and androgen receptor co-activation (e.g., P2RX1, RASAL1, DUSP6, TGFB1I1, SLAMF7) (FIG. 17f). Overall, these results indicate that modulating TGFβ1/BMP-signals re-wires transcriptional circuits of Dysf. CD8+ T cells to recover effector function and memory gene expression programs, while modulating multiple signaling programs with new roles in the biology of T cell dysfunction.

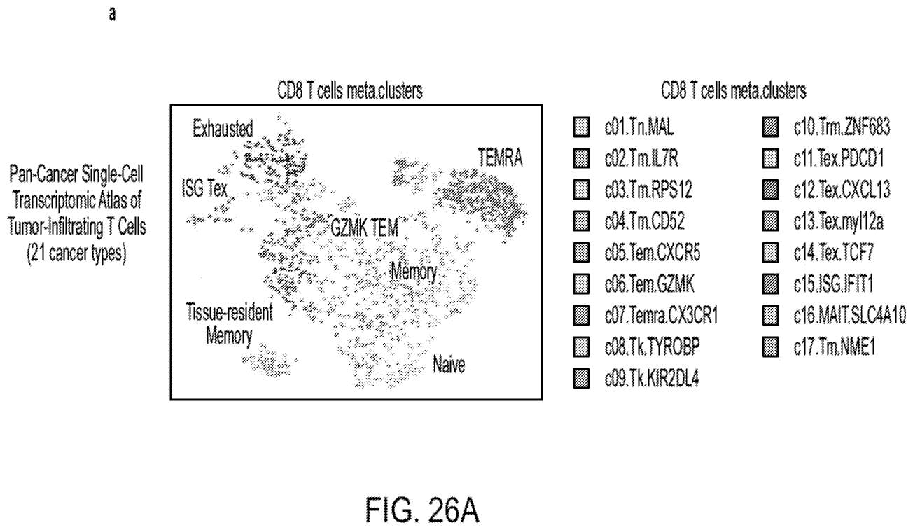

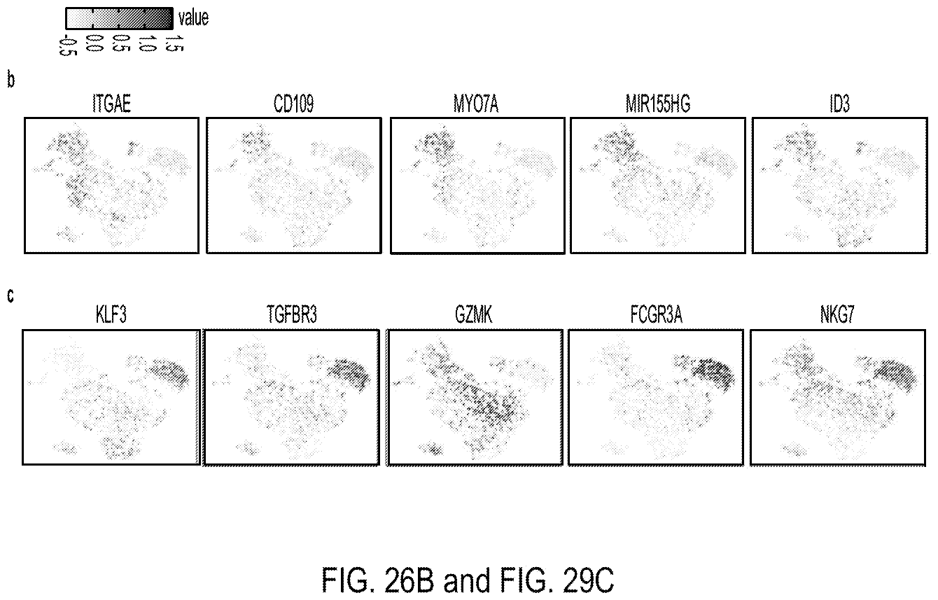

Next, we sought to determine whether the observed transcriptional changes among ex vivo-differentiated CD8+ T cells recapitulate gene expression signatures of comparable subsets isolated from patients with cancer. We performed over-representation analysis (ORA) using a comprehensive single-cell transcriptomic database of naïve, memory, and dysfunctional human CD8+ T cell clusters that includes single-cell RNA-seq datasets from >300 patients across 21 different types of cancer. This analysis revealed a significant enrichment of chronic TGFβ1-associated gene signature (i.e., upregulated genes in Dysf. T cells) within the transcriptional signatures of tumor-infiltrating terminally exhausted and OXPHOS^{-ve} exhausted CD8+ T cell clusters (FIG. 17h). Indeed, Dysf. CD8+ T cells recapitulated many unique gene expression programs in exhausted human tumor-infiltrating CD8+ T cells (TILs), such as ITGAE, CD101, CD109, MYO7A, MIR155HG (FIG. 24e, FIG. 25k-n; FIG. 26a,b). In contrast, recovered genes in Reprogrammed CD8+ T cells were significantly enriched for the transcriptional signatures of specific functional CD8+ T cell clusters, such as TEMRA and ZNF683+ CXCR6− memory T cells (FIG. 17i, FIG. 26c). These data further demonstrate that prolonged TGFβ1 exposure establishes a transcriptional landscape in chronically stimulated CD8+ T cells similar to that of terminally dysfunctional CD8+ T cells in human cancers, which can be effectively reversed into a functional memory-like state by rebalancing TGFβ1/BMP-signals.

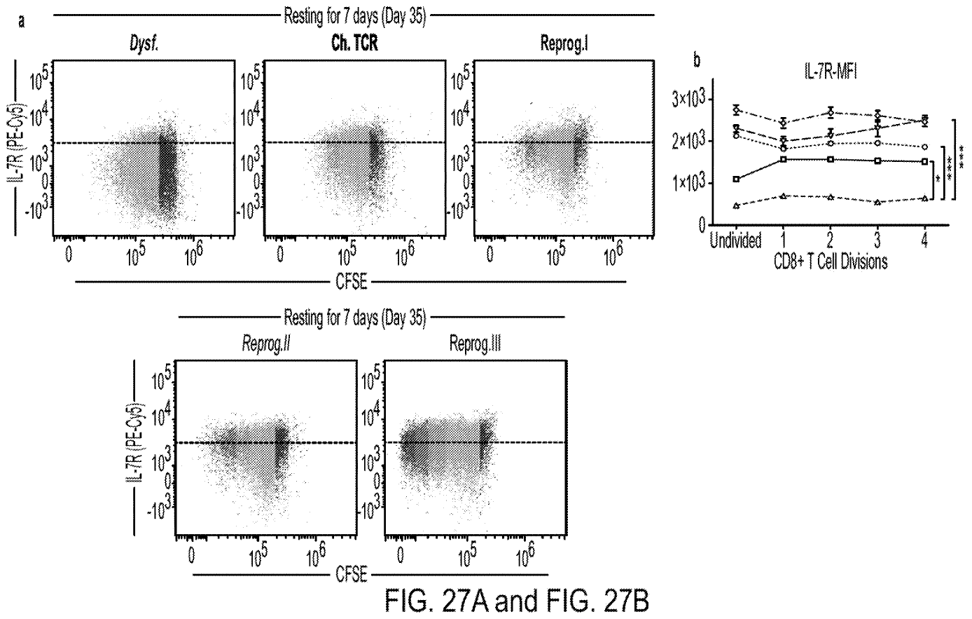

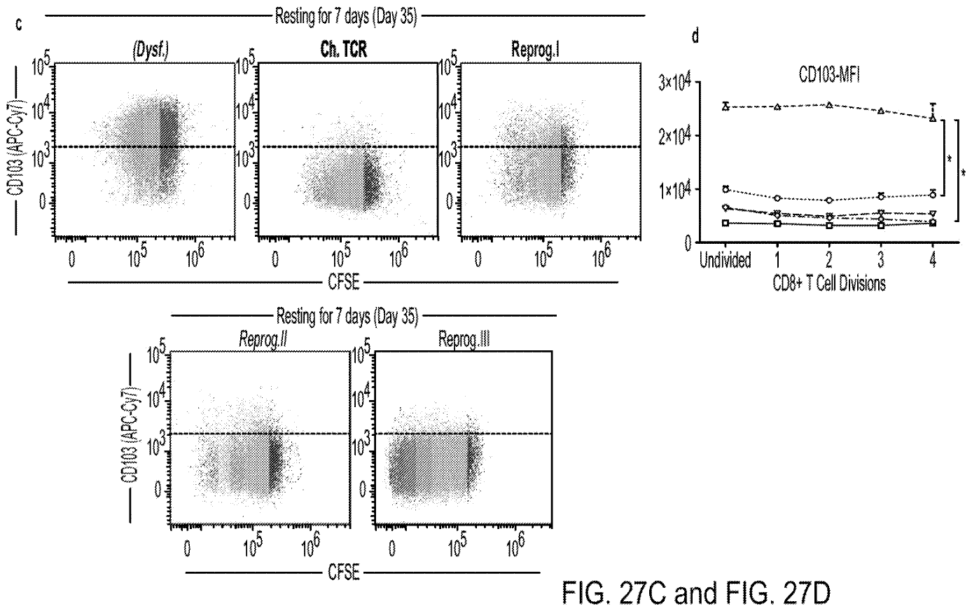

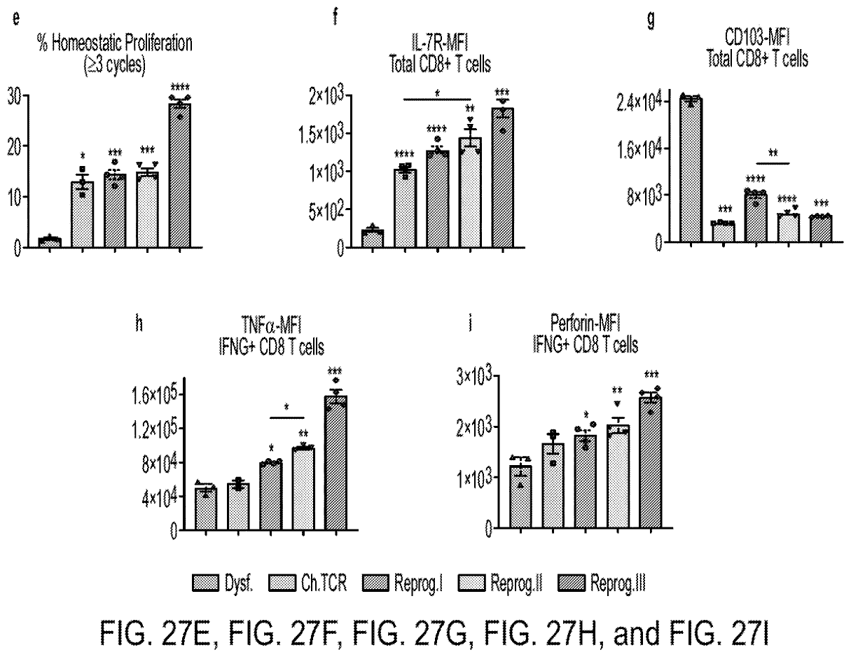

We next examined whether Reprogrammed CD8+ T cells stably maintain a functional memory-like state after cessation of treatment. Specifically, we tracked changes in effector functions, memory, and self-renewal capacity in Reprogrammed or Dysf. CD8+ T cells for ≥7 days under homeostatic conditions (see FIG. 14a). Reprogrammed CD8+ T cells underwent efficient homeostatic proliferation while maintaining significantly higher expression levels of effector cytokines and memory genes (e.g., IL-7R, TNFα, Perform) relative to Dysf. T cells (FIG. 27). In contrast, Dysf. CD8+ T cells maintained higher expression of CD103 (encoded by ITGAE) (FIG. 27c-d, 21g), indicating an important role for TGFβ1 signals in driving a stable transcriptional program commonly found in both tumor-infiltrating human dysfunctional and tissue-resident memory CD8+ T cells. Taken together, these findings reveal a novel role for TGFβ1/BMP signals in the transcriptional (re)programming of terminally dysfunctional CD8+ T cells that can be therapeutically targeted to restore a functional memory-like state.

### (6) Epigenetic Remodeling of Dysfunctional Human T Cells by Targeting TGFβ1 and Boosting BMP Signals

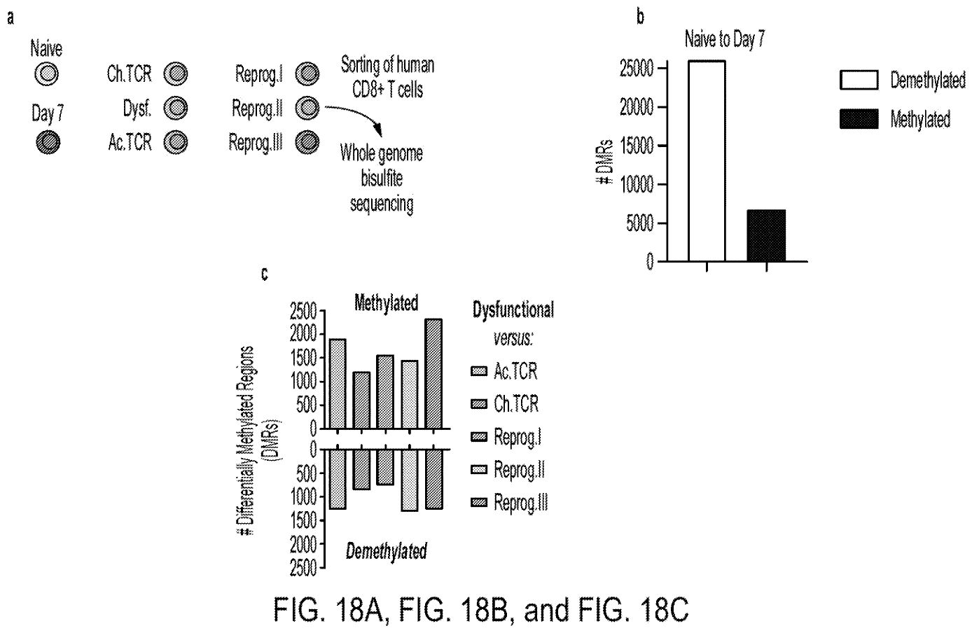

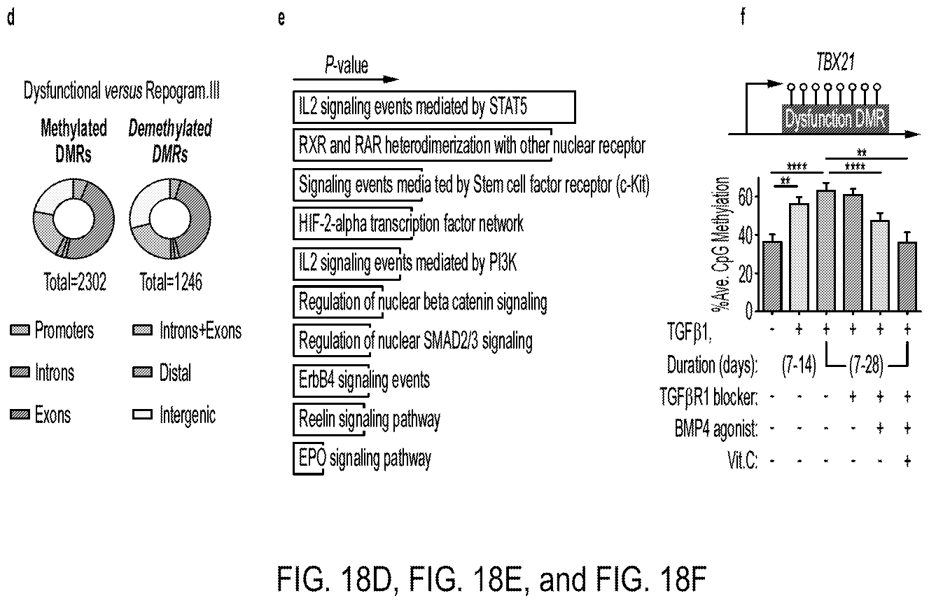

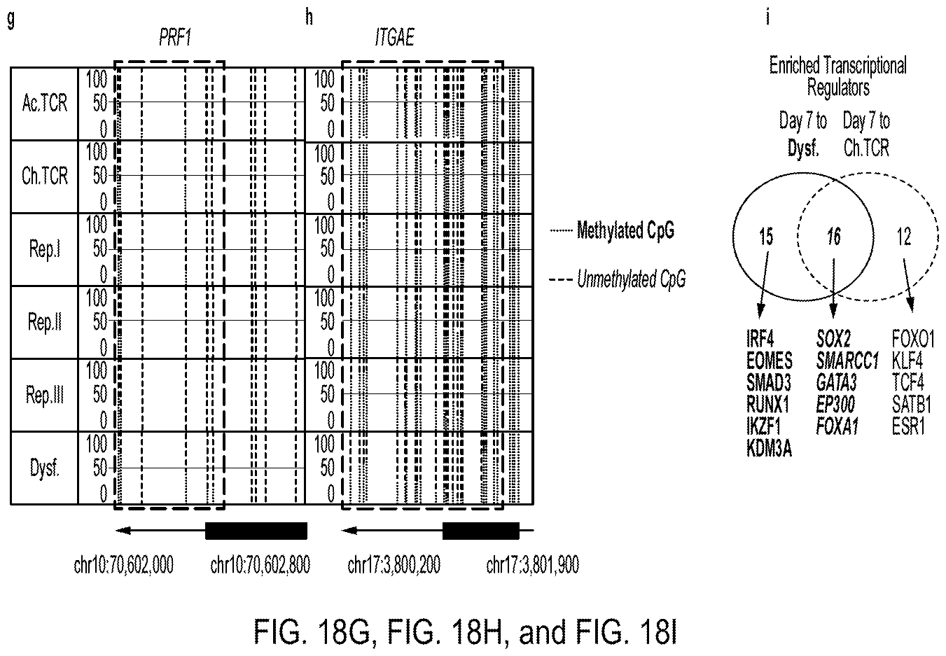

The remarkable stability of functional impairment observed in resting Dysf. CD8+ T cells prompted us to investigate whether transcriptional rewiring during chronic TGFβ1 plus TCR stimulation is coupled with epigenetic remodeling in human CD8+ T cells. DNA methylation is a fundamental epigenetic mechanism that enforces terminal dysfunction in chronically stimulated CD8+ T cells. To assess this process under distinct microenvironmental stimulation conditions, we measured global DNA methylation changes in isolated human CD8+ T cells using whole-genome bisulfite sequencing (WGBS) (FIG. 18a). Similar to global DNA methylation changes during effector differentiation in mice, we found that, during naïve-to-day 7 transition, acutely stimulated human CD8+ T cells underwent extensive DNA demethylation with >25,000 differentially methylated regions (DMRs) (FIG. 18b). Following these initial demethylation events, Dysf. CD8+ T cells acquired a distinct DNA methylation landscape with more hypermethylated DMRs relative to chronically or acutely TCR stimulated CD8+ T cells. Importantly, RepSox treatment induced distinct methylation patterns in Dysf. CD8+ T cells, which became more pronounced when combined with the BMP4 agonist and vitamin C treatment (FIG. 18c). These patterns were mainly detected within gene bodies and putative gene regulatory regions, such as introns and distal regions (FIG. 18d), indicating a role of these epigenetic changes in regulating gene expression programs. Indeed, many hypermethylated programs were acquired in Dysf. CD8+ T cells at effector or memory T cell-associated loci (e.g., PRF1, GZMK, CD28, TBX21, and TCF7), and enriched in pathways regulating T cell function and sternness, such as IL-2 signaling, stem cell factor receptor signaling, and nuclear beta-catenin signaling (FIG. 18e). Moreover, targeted epigenetic analysis confirmed that chronic TGFβ1 signaling accelerates de novo DNA methylation within the TBX21 locus an epigenetic process that can be reversed only by combined TGFBR1 blockade and BMP4 agonist treatment of Dysf. CD8+ T cells (FIG. 18f). Coupled with these methylation programs, Dysf. CD8+ T cells also underwent distinct demethylation events in genes characteristic of human tumor-infiltrating CD8+ T cells, such as ITGAE (FIG. 18g,h). These data indicate that the stable, terminally dysfunctional state in human CD8+ T cells is driven, in part, by DNA methylation reprogramming that can be remodeled by modulating TGFβ1/BMP signals.

While DNA (de)methylation is imprinted by known enzymatic machinery (e.g., DNMT1/3A/3B, TET enzymes), it remains unclear what factors instruct this (re)programming process. To gain insights into TF networks that regulate dysfunction-associated epigenetic programs, we utilized a comprehensive pool of published ChIP-seq datasets for human TFs and performed enrichment analysis for transcriptional regulators with enriched binding within genomic regions that were demethylated during day 7-to-day 28 transition in chronically stimulated CD8+ T cells. We found a common enrichment of some TF binding elements (e.g., SOX2, GATA3, EP300) among chronically stimulated CD8+ T cells in the presence or absence of chronic TGFβ1 signals (FIG. 18i). Importantly, this analysis also showed an enrichment for unique transcriptional regulators in Dysf. CD8+ T cells; some of are key regulators of the dysfunctional T cell's biology (e.g., IRF4, EOMES), while others are new regulators, such as IKZF1, SMAD3, and KDM3A (FIG. 18i). Conversely, Dvsf. CD8+ T cells lost multiple binding motifs that were only enriched in chronically TCR stimulated CD8+ T cells, including TFs known to regulate T cell stemness and cytokine signaling, such as FOXO1, KLF4, TCF4, and SATB1 (FIG. 18i). Collectively, these findings reveal a novel role of chronic TGFβ1 signals in re-routing the epigenetic landscape within chronically stimulated human CD8+ T cells, enforcing their fate commitment to dysfunction. Importantly, targeting TGFβ1, coupled with the augmentation of BMP signals and vitamin C treatment, can epigenetically reprogram Dysf. CD8+ T cells.

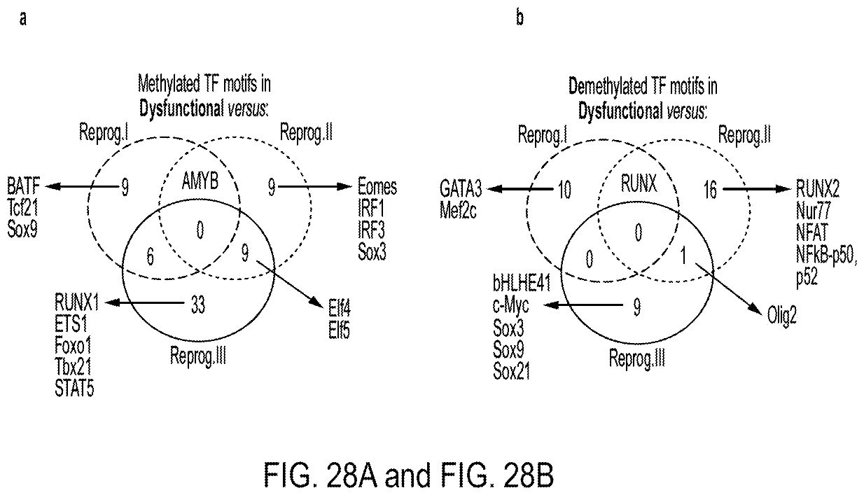

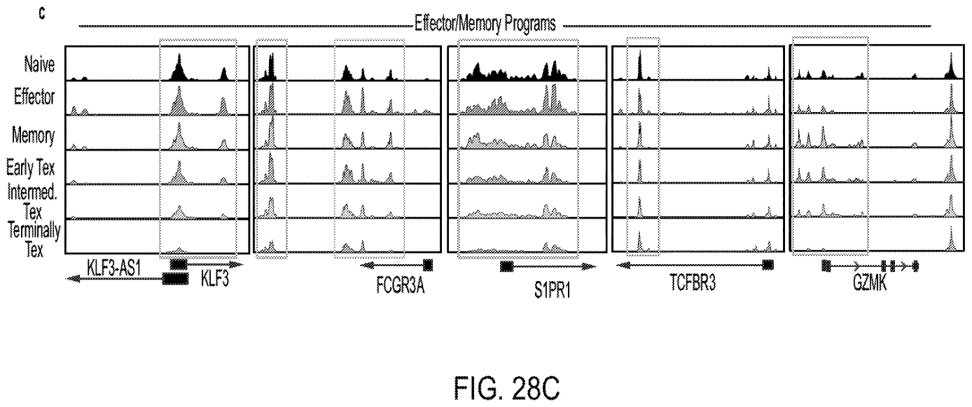

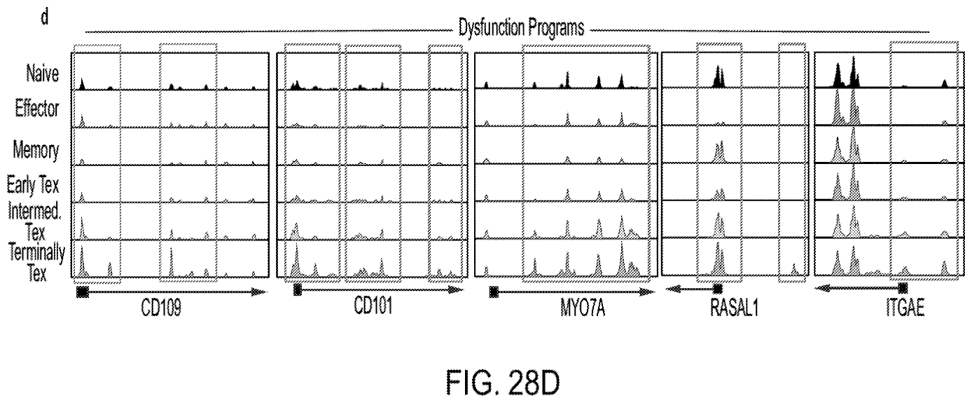

To further understand the molecular mechanisms underlying the BMP signaling-driven reprogramming of dysfunctional CD8+ T cells, we performed motif enrichment analysis of DMRs that were hypomethylated in BMP agonist-treated reprogrammed cells. We found significant enrichment of new TFs, such as the ELF4-KLF2 axis, which is a key regulator of naïve CD8+ T cell quiescence and memory T cell function, in addition to key TFs regulating effector and memory T cell differentiation (e.g., EOMES, RUNX1, TBX21, IRF1) (FIG. 28a). This motif enrichment in DMRs was coupled with boosting the expression of key TFs and chemokine receptors that regulate function and chemotaxis of memory and cytolytic CD8+ T cell subsets (e.g., KLF2, KLF3, SOX4, CX3CR1, CXCL8, S1PR1) (FIG. 25f). Conversely, genomic regions that are hypomethylated in Dysf. Versus reprogrammed II or III CD8+ T cells showed significant enrichment with binding motifs of TCR signaling regulators (e.g., Nur77, NFAT) and new TFs (e.g., Olig2, Sox3, Sox9) (FIG. 28b).

Furthermore, we examined chromatin accessibility changes at genes differentially regulated in Dysf. Versus Reprogrammed II CD8+ T cells using a single-cell open chromatin (scATAC-seq) database of human naïve, effector, memory, and exhausted CD8+ T cell subsets from patients with cancer. Importantly, we found concordant changes in chromatin accessibility among effector, memory, or progenitor Tex subsets of CD8+ T cells, with changes in reprogramming-associated DNA methylation and gene expression programs (i.e., increased accessibility at upregulated and/or hypomethylated genes), such as KLF3, FCGR3A, S1PR1, TGFBR3, and GZMK (FIG. 28c). Conversely, we observed increased chromatin openness in terminally exhausted TILs at genes upregulated in Dysf. CD8+ T cells, including novel genes with unknown functions in exhausted T cell biology, such as CD109, CD101, MYO7A, RASAL1, and ITGAE (FIG. 28d). Overall, BMP-driven transcriptional and epigenetic changes in reprogrammed T cells revealed BMP modulatory effects on multiple signaling programs and stemness/memory-related transcriptional circuits, with new roles in the biology of T cell dysfunction and reprogrammability of dysfunctional cells towards an effector/memory-like state.

### (7) Reprogrammed CD8+ T Cells Exhibit Superior Anti-Tumor Cytotoxic Activity

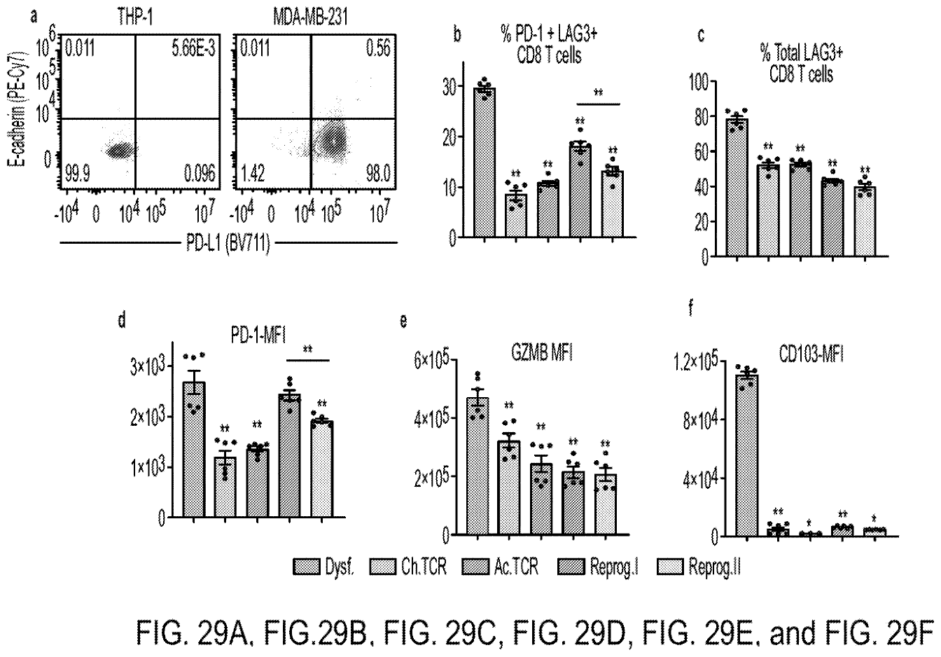

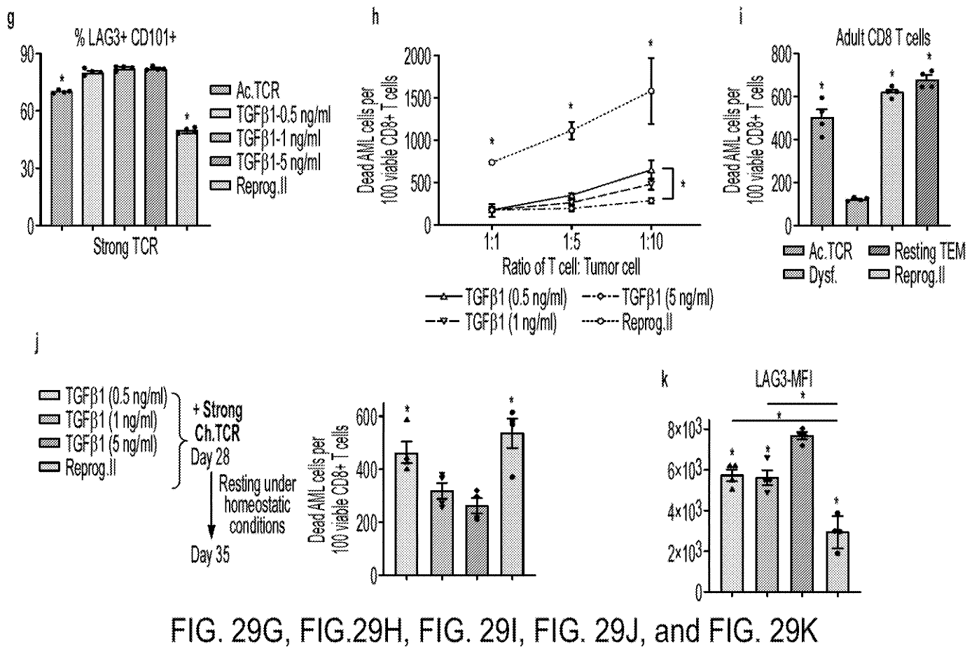

To assess the cytotoxic function of our ex vivo-differentiated human CD8+ T cells, we developed a new TCR-dependent tumor killing assay. In brief, we co-cultured T cells with human acute myeloid leukemia (AML) cells in the presence of anti-CD3 antibody, which coats the tumor cells via binding to Fc receptors and, in turn, cross-links the T cell co-receptor (FIG. 19a; FIG. 29a). We found that acutely or chronically TCR stimulated CD8+ T cells maintained significantly elevated cytotoxicity against AML cells relative to Dysf. CD8+ T cells. Furthermore, therapeutically reprogrammed CD8+ T cells recovered a striking anti-tumor killing activity, with superior cytotoxicity relative to acutely or chronically TCR-stimulated CD8+ T cells (FIG. 19b). To better understand anti-tumor CD8+ T cell responses, we measured phenotypic changes in human CD8+ T cells after 18 hours of tumor co-culture. We found that Dysf. CD8+ T cells upregulated the highest levels of the inhibitory receptors LAG3 and PD-1, GZMB, CD103 and CD101; markers which distinguish the most dysfunctional subset of CD8+ T cells during chronic infections or cancer. In contrast, Reprogrammed II CD8+ T cells expressed the lowest levels of CD101, further indicating a key role for BMP signals in driving T cell's fate away from terminal dysfunction (FIG. 19c-e, FIG. 29b-f).

Metastatic tumors usually succeed in evading the host's surveillance mechanisms after epithelial-mesenchymal transition, a process often characterized by loss of E-cadherin. In addition, some tumors resist CD8+ T cell cytotoxicity by expressing high levels of ligands, such as PD-L1, for inhibitory receptors on activated CD8+ T cells. We sought to assess the anti-tumor killing capacity of chronically stimulated CD8+ T cells against the immunotherapy-resistant tumor line MDA-MB-231, a metastatic breast adenocarcinoma that expresses high basal levels of PD-L1 (FIG. 29a). Reprogrammed CD8+ T cells restored a significantly stronger killing activity against the breast adenocarcinoma cells relative to Dysf. or chronically stimulated T cells (FIG. 19f). Similarly, under persistent strong TCR stimulation settings, Reprogrammed II CD8+ T cells restored a superior killing activity of breast adenocarcinoma or AML cells, and maintained this enhanced cytotoxicity on day 36 following rest in homeostatic conditions for >1 week (FIG. 19g-h, FIG. 29g-k). Furthermore, reprogrammed adult CD8+ T cells under strong TCR stimulation conditions recovered a potent cytotoxicity against AML, to levels comparable to chronically stimulated adult effector memory (TEM) CD8+ T cells (FIG. 19i). These data indicate that therapeutic rebalancing of TGFβ1/BMP signaling can be of clinical importance for restoring human CD8+ T cell activity against immunotherapy-resistant human tumors.

To validate the in vitro human T cell cytotoxicity data in an antigen-specific manner, we developed another model system of T cell dysfunction in mouse TCR-transgenic P14 (expressing gp33 epitope-specific TCR) CD8+ T cells during persistent tumor challenge. We induced T cell dysfunction via repeated coculturing of P14 cells with gp33-expressing CT2A glioma cells after the effector differentiation of naïve P14 cells, followed by treatment regimens, including RepSox (TGFβR1 blocker), BMP4 agonist, or combined therapy (FIG. 19j). Similar to the human T cell cytotoxicity results, P14 cells treated with combined TGFβR1i+ BMP4a showed enhanced killing of CT2A tumor cells, while maintaining polyfunctionality (FIG. 19k, 19l) compared to vehicle or monotherapy conditions. Furthermore, treated P14 cells had reduced expression of inhibitory receptors and CD103, indicating an activation of endogenous TGFβ1 signaling in chronically stimulated P14 cells that is modulated by TGFβR1 blockade and BMP4 agonist treatments (FIG. 19m, 19n). We next validated the therapeutic benefits of modulating TGFβ1/BMP signaling during cancer in vivo. We used the B16-F10 (expressing LCMV-GP) melanoma model and assessed the effect of mono- or combined TGFβR1i and BMP4a therapies initiated after tumor development (FIG. 19o). Importantly, while a modest tumor control was observed in monotherapy-treated animals, we found a significantly elevated control of tumors in animals treated with combined TGFβR1i plus BMP4a therapy (FIG. 19p). These in vivo data further confirm the therapeutic benefits of rebalancing TGFβ1/BMP signaling for treating cancer.

### (8) Combined RepSox and BMP4 Agonist Boosts the ICB Response of Exhausted CD8+ T Cells

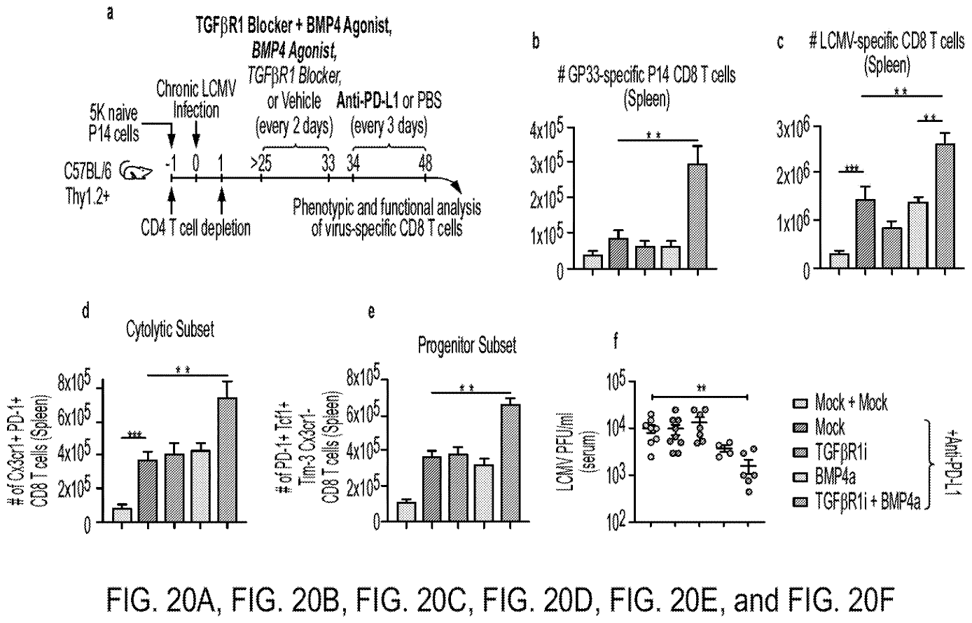

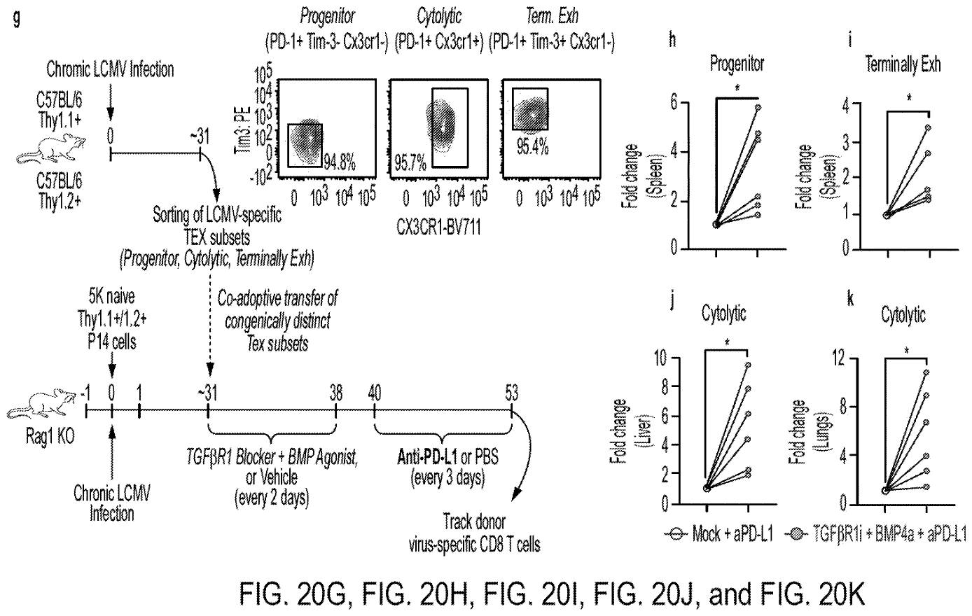

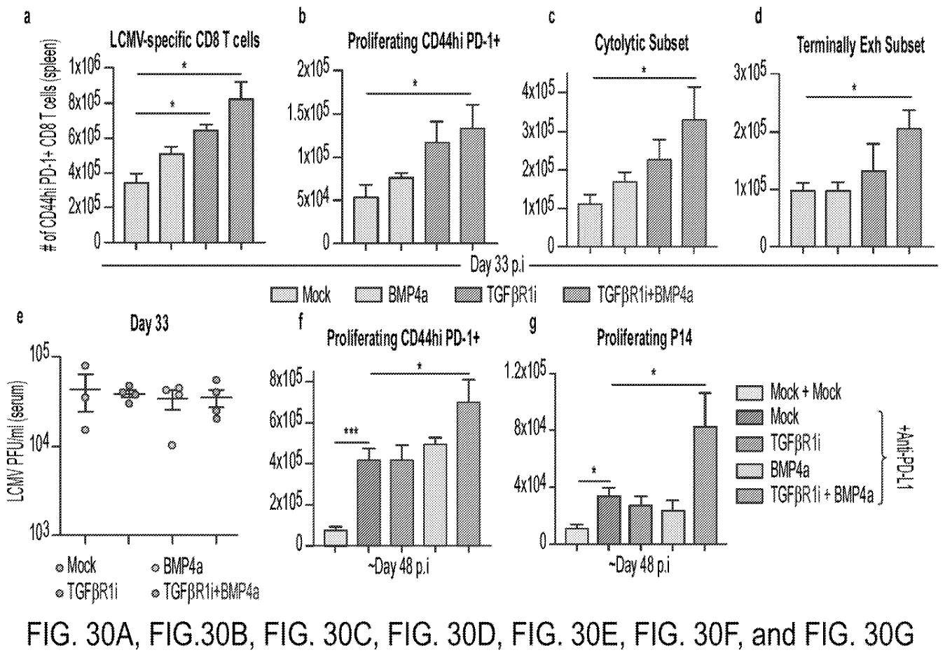

Terminally dysfunctional CD8+ T cells fail to respond to ICB therapy in many preclinical models of T cell dysfunction and human cancers. While selective targeting of TGFβ1 signaling can improve infiltration and/or function of CD8+ T cells, studies have mainly used genetic disruption or attenuation of TGFβ1 signaling in naïve CD8+ T cells prior to their differentiation into a terminally dysfunctional state. Thus, the observed improvement in CD8+ T cell responses likely arises due to an enhanced priming and/or Cxcr3-mediated migration of early differentiated CD8+ T cells. To determine whether our new therapeutic strategy can reprogram terminally dysfunctional CD8+ T cells and rescue their response to ICB therapy in vivo, we tested the efficacy of late RepSox treatment in a preclinical model of severe T cell exhaustion, namely, CD4− helpless lifelong chronic LCMV infection (FIG. 20a). As shown in FIG. 20b-e, single RepSox or BMP4 agonist treatment did not improve dysfunctional CD8+ T cell responses during anti-PD-L1 treatment. However, combined RepSox and BMP4 agonist therapy synergized virus-specific CD8+ T cel 1 responses, with striking increases in the quantity of both polyclonal LCMV-specific (CD44+PD-1+ CD8+) and GP33 antigen-specific P14 CD8+ T cells after ICB treatment (FIG. 20b-c). The enhanced T cell responses were observed across all main subsets of dysfunctional virus-specific CD8+ T cells, particularly the stem-like progenitor (CD44+PD-1+ Tim-3−) and cytolytic (CD44+PD-1+Cx3cr1+) subsets (FIG. 20d-e). The improved responses can be mediated by enhanced proliferation of dysfunctional virus-specific CD8+ T cells (FIG. 30b, 30f, and 30g) and/or reprogramming of ICB-refractory subsets towards an ICB-responsive state with no significant changes observed in Treg numbers. Importantly, we measured viral titers in serum samples collected from LCMV-infected mice after the indicated treatment regimens (FIG. 20a) and found enhanced viral control after combined TGFβR1i plus BMP4a therapy followed by PD-L1 blockade (FIG. 20f), indicating boosted anti-viral responses by reprogrammed CD8+ T cells. This finding is significant given that treatments were given to a lifelong chronic infection model with a severe level of T cell exhaustion, in which ICB does not normally show an enhanced anti-viral response. In contrast. BMP4a (or TGFβR1i) monotherapy did not induce significant improvement in CD8+ T cell responses. Such lack of effector recovery under BMP4a monotherapy can be due to a disproportionate use of the common SMAD4 and/or induced upregulation of negative regulators of SMAD1/5 signaling (e.g., SMAD6-FIG. 25n) during chronic TGFβ1 signals, leading to a limited activation of BMP-driven SMAD1/5 signaling in the absence of TGFβR1 blockade. We also assessed the effect of mono- or combined TGFβR1i and BMP4a therapies without PD-L1 blockade during CD4-helpless chronic LCMV infection. Importantly, only combined TGFβR1i and BMP4a treatment significantly boosted the quantity of both cytolytic and terminally exhausted subsets of virus-specific CD8+ T cells (FIG. 30a-d).

To dissect the responses of different exhausted T cell subsets after TGFβR1 blocker plus BMP4 agonist therapy, we co-adoptively transferred congenically distinct subsets of LCMV-specific exhausted T cells (progenitor, cytolytic, and terminally exhausted=“CD44+PD-1+ Tim-3+Cx3cr1−”) into infection-matched Rag1 KO animals, followed by treating the recipient animals with the indicated treatment regimens (FIG. 20g). Combined TGFβR1 blockade and BMP4 agonist significantly boosted the rejuvenation of all subsets of exhausted T cells after PD-L1 blockade, with the most pronounced fold-change observed in the cytolytic subset within secondary lymphoid and peripheral non-lymphoid tissues (FIG. 20h-k). Collectively, we conclude that resetting the balance between TGFβR1 and BMP signals can reverse the fate commitment and ICB-refractory state of severely dysfunctional CD8+ T cells, thereby boosting the efficacy of ICB therapy.

### b) DISCUSSION

TGFβ1/BMP signals control numerous cell fate decisions in a context-dependent manner during tissue development and homeostasis. Our study defines a new and critical role for TGFβ1/BMP signals as key determinants for epigenetic (re)programming and cell fate commitment of dysfunctional CD8+ T cells. Recent work has shown that effector T cells retain functional capacity and memory potential when transferred to an antigen-free environment, while post-effector epigenetic changes during chronic infections or cancer enforce terminal dysfunction of CD8+ T cells; thereby restraining their ICB response, and perhaps explaining why ICB therapy fails in some cancer patients. In our study, we addressed a fundamental question about the role of microenvironmental signals in epigenetic regulation of chronically stimulated CD8+ T cells at the post-effector stage. We show that persistent, simultaneous TGFβ1 plus TCR signaling, rather than continuous TCR stimulation alone, establishes a stable, terminally dysfunctional state in human CD8+ T cells.

A recent study reported that chronic antigenic exposure in vitro induces dysfunction of human chimeric antigen receptor (CAR) T cells. However, the chronically stimulated CAR T cells retained a high capacity to secrete effector cytokines upon in vitro activation with PMA/ionomycin. The reversible dysfunction phenotype observed in these CAR-T cells can be explained, in part, by the ability of PMA/ionomycin to bypass defects in upstream events of the antigen-induced signaling process. Moreover, after terminating chronic antigenic stimulation, dysfunctional CAR T cells rapidly recovered their cytotoxic function (within 24 hours), when cultured under homeostatic conditions, further implying that chronic antigenic stimulation is insufficient to enforce a heritable dysfunctional program in CAR T cells. In another recent study, consistent with our findings, T cell-intrinsic TGFβ1 signaling was reported to accelerate dysfunction of murine virus-specific CD8+ T cells during chronic LCMV infection. Yet, early TGFβ1 signaling in effector CD8+ T cells was shown to support the preservation of progenitor dysfunctional T cells by preserving their cellular metabolism. In this regard, we have shown that TGFβ1 signals support the survival of CD8+ T cells under persistent strong TCR stimulation. Yet, the ultimate outcome is that the surviving CD8+ T cells eventually progress to a terminal dysfunctional state. As post-effector chronic TGFβ1 exposure remodeled DNA methylation and transcriptional programs of chronically stimulated human CD8+ T cells, these events coordinated the development of a terminal dysfunctional state that recapitulated many unique aspects of those found in tumor-infiltrating dysfunctional CD8+ T cells across multiple types of cancer. Importantly, we demonstrated that terminal dysfunction is stabilized by epigenetic remodeling within the epigenetically flexible effector, rather than memory, CD8+ T cells when subjected to chronic TGFβ1 signals. These results demonstrate that the development of chronic TGFβ1-mediated T cell dysfunction is epigenetically regulated in a context-dependent manner, rather than via a strictly immunosuppressive effect. Overall, these findings highlight the insufficiency of chronic TCR stimulation in driving stable T cell dysfunction, and underscore how timing of TGFβ1 signaling regulates differential functional fates.

BMP ligands, which are members of the TGFβ super-family, have been implicated as key regulators in the development and maintenance of multiple tissues, such as bones, lymphatics, and blood vasculature. Moreover, the BMP4 ligand regulates the developmental program and stemness of hematopoietic and embryonic stem cells. We now show that BMP signals counteract TGFβ1-driven terminal dysfunction of CD8+ T cells. Indeed, targeting of TGFβ31 while augmenting BMP signals effectively reprogrammed terminally dysfunctional CD8+ T cells to rescue effector functions, as well as their underlying transcriptional and epigenetic states. Although we have not observed any signs of bystander activation of CD8+ T cells, it remains to be determined whether this treatment regimen has modulatory effects on auto-reactive or memory CD8+ T cell responses. Importantly, our therapeutic strategy also re-activated memory programs in dysfunctional T cells, opening the possibility of promoting durability in the rejuvenated cells.

At the practical level, we have developed a tractable in vitro model system that facilitates mechanistic investigations of human T cell dysfunction and can guide or complement in vivo studies. As an example, the therapeutic approach that emerged from in vitro experiments was a rebalancing of TGFβ1/BMP-signals. When applied to a preclinical animal model of severe T cell exhaustion, lifelong LCMV infection, we observed a significant boost in CD8+ T cell responses to PD-L1 blockade therapy leading to a remarkable decrease in viral titers. Likewise, this therapeutic approach rescued the anti-tumor function of terminally dysfunctional human CD8+ T cells toward both hematopoietic and solid, metastatic cancer lines, as well as enhanced tumor control in mice. Yet, this in vitro model remains to be optimized to better dissect the impact of antigen-TCR binding in T cell dysfunction using antigen-specific, rather than polyclonal, naïve precursor human CD8+ T cells. Taken together, these new findings indicate that chronic TGFβ1 signaling enforces the development of terminal T cell dysfunction, a process that can be therapeutically targeted to epigenetically revive effector and memory programs to treat cancer or chronic viral infections.

### c) Materials and Methods

(1) CBMC Isolation from Human Cord Blood

Whole human cord blood samples from healthy anonymous donors were obtained through the Leukemia Tissue Bank at the Ohio State University Comprehensive Cancer Center under the IRB-approved protocol number OSU-1997C194. Cord blood mononuclear cells (CBMCs) were isolated from whole blood by centrifugation with Ficoll-Paque PLUS (Cytiva) and processed under our approved IBC protocol #2020R00000129.

(2) PBMC Isolation from Human Adult Blood

Source leukocyte buff, coat samples from healthy anonymous donors were received from the Gulf Coast Regional Blood Center in Houston, TX. Peripheral blood mononuclear cells (PBMCs) were isolated from the samples by centrifugation with Ficoll-Paque PLUS and processed under our approved IBC protocol #2020R00000129.

### (3) In Vitro Stimulation of Human CBMCs

Isolated human CBMCs were seeded into 96-well plates (100,000 viable cells per well) in complete RPMI 1640 medium (with 10% FBS, 1× Penicillin/Streptomycin) (Gibco), containing 40 IU/ml of recombinant human IL-2 (Peprotech), 10 ng/ml of recombinant human IL-15 (Peprotech). For “Weak TCR” stimulation, 30 ng/ml of purified monoclonal anti-human CD3 (clone: OKT3; BioLegend) was continuously added onto the seeded CBMCs to provide TCR stimulation plus natural costimulation via CD80/CD86 on Fc-expressing CBMCs that survive in the first few days. For “Strong TCR” stimulation setting, flat-bottom plates were coated with purified monoclonal anti-human CD3 (5 ug/ml) and anti-human CD28 (1 ug/ml) (clone: CD28.2; BioLegend) at 4° C. overnight, then CMBCs were isolated and seeded in complete T cell medium as described above on day 0. On day 7, viable CD8+ T cells were FACS-purified using the Sony MA900 cell sorter. Purified CD8+ T cells were then continuously stimulated in plates coated with anti-CD3 only (5 ug/ml). Media was replenished every 2-3 days, and cells were incubated at 37 C and 5% CO2 throughout the duration of the experiment. Human TGFβ1 (5 ng/ml, Peprotech) was added to the media starting on day 7. Treatment with RepSox (25 μM) and SB4 (10 μM) (MedChemExpress LLC) or vehicle control (DMSO, ThermoFisher) were added to the media starting on day 14 or 21. Vitamin C (100 μM, MedChemExpress LLC) was added to the media starting on day 21. For the acute TCR stimulation condition, anti-CD3 was no longer added to the media starting on day 7. For recovery experiments in FIG. 16 and FIGS. 22 and 26, CD8+ T cells were rested from TCR stimulation and TGFβ1 starting on day 28 for 7-14 days in T cell media containing recombinant human IL-15 only (10 ng/ml).

### (4) In Vitro Stimulation of Human Adult PBMCs

Isolated adult PBMCs were stained on day 0 for cell sorting of naïve CD8+ T cells (viable CD3+ CD8+ CCR7+ CD45RO−) and viable CD3− PBMCs using the Sony MA900 sorter. The cells were seeded into 96-well U-bottom plate (˜5,000 naïve T cells+45,000 CD3− cells per well) on day 0 with 30 ng/ml of purified anti-human CD3, in complete T cell medium as described above. On day 4, the wells were transferred to a 96-well flat-bottom plate coated with anti-human CD3 and CD28 antibodies as described above, and stimulation was continued for an additional three days. On day 7, the cells were returned to culture with continuous stimulation using soluble anti-CD3 (30 ng/ml) or repeated plate-bound anti-CD3 stimulation through day 28. Media was replenished every 2-3 days. Treatments were added to the media as described above for CBMCs experiments.

### (5) Tumor Cell Lines for Human T Cell Cytotoxicity Assays

Human acute monocytic leukemia (AML) THP-1 cell line was a gift from Dr. Amal Amer at the Ohio State University. THP-1 cells were cultured in complete RPMI 1640 medium (with 10% FBS, 1× Penicillin/Streptomycin), 10 mM HEPES buffer and 1 mM sodium pyruvate (Gibco) at 37° C. and 5% CO₂. MDA-MB-231 tumor cell line was a gift from Dr. Gina Sizemore at the Ohio State University. MDA-MB-231 cells were cultured in complete RPMI 1640 medium (with 10% FBS, 1× Penicillin/Streptomycin) at 37° C. and 5% CO₂. All cell lines were authenticated and regularly tested for Mycoplasma.

(6) In Vitro Stimulation of Mouse P14 Cells and Co-Culture with Tumor Cells

Spleens were harvest from naïve P14 mice and viable naïve P14 cells (GP33− specific CD8+ T cells) were purified using negative enrichment kit for naïve mouse CD8+ T cells (STEMCELL—EasySep). Naive P14 cells were seeded into 96-well U-bottom plates (120,000 viable cells per well) in complete RPMI 1640 medium (Gibco) with 10% FBS, 1× Penicillin/Streptomycin, containing 80 IU/ml of recombinant human IL-2 (Peprotech) and 10 ng/ml of recombinant human IL-15 (Peprotech). Then, P14 cells were stimulated ex vivo using GP33 peptide (0.2-0.3 ug/ml) for 6 days with replenishment of media plus peptide every 2 days. On day 6, effector differentiation and expansion of P14 cells was confirmed (e.g., high expression of CD44, IFNγ, TNFα, Gzmb, T-bet), after which effector P14 cells were chronically stimulated via repeated coculture with GP33− expressing CT2A glioma tumor cells every 1-2 days. Treatment with RepSox (25 μM) and SB4 (10 μM) (MedChemExpress LLC) or vehicle control (DMSO, ThermoFisher) were added to the media starting on day 7 with replenishment every 1-2 days until day 16-19. Cells were incubated at 37° C. and 5% CO2 throughout the duration of the experiment.

### (7) Mouse Chronic LCMV Infection Model

Healthy, wild-type 8 week-old C57BL/6 mice were purchased from Jackson laboratory. The P14 mouse strain was a kind gift from Dr. Rafi Ahmed at Emory University. and blood-circulating CD8+ T cells were confirmed to have TCR-specific to the LCMV glycoprotein antigen GP33 and express the Thy1.1 congenic marker. 5,000 naïve Thy1.1+P14 cells were adoptively transferred into Thy1.2+C57Bl/6 mice one day prior to LCMV infection. Recipient mice were treated with GK1.5 antibody (Harlan Bioproducts; 500 μg/mouse, i.p injection) to deplete CD4+ T cells on days −1 and +1 during viral infection. On day 0, recipient tice were infected with Clone 13 chronic LCMV (2×10×6 pfu/mouse, i.v. injection). Starting ˜day 25-27, mice received either Vehicle (DMSO in PBS), RepSox (TGFβR1 inhibitor, 5 mg/Kg) or RepSox+SB4 (BMP-4 agonist, 5 mg/Kg) via i.p. injection every 2 days for 8 days. Following these treatments starting day ˜34-36, mice received monoclonal anti-PD-L1 treatment (BioXCell, 200 μg/mouse) or PBS via i.p. injection every 3 days for 5 doses. Within 2 days after the final anti-PD-L1 dose, mice were euthanized and blood, spleens, livers, and lungs were harvested and processed as previously described¹⁵. LCMV viral loads were determined from serum of infected mice by the plaque assay performed using Vero cells. For the co-adoptive transfer experiments described in FIG. 20g, healthy, wild-type 8-week-old C57Bl/6 mice (Thy1.1+ or Thy1.2+) were infected with Clone 13 chronic LCMV. Concurrently, Rag1-KO mice received 5,000 naïve Thy1.1+/1.2+P14 cells followed by chronic LCMV infection as described above. On day ˜31 post-infection, LCMV-specific CD8+ T cells (CD44+ PD-1+) were isolated from spleens of infected C57Bl/6 mice and sorted into three subsets: (1) Progenitor (CD44+ PD-1+ Tim3− Cx3cr1−). (2) Cytolytic (CD44+PD-1+Cx3cr1+), and (3) Terminally Exhausted (CD44+PD-1+ Tim3+Cx3cr1−). Congenically distinct subsets were co-adoptively transferred into infection-matched Rag1-KO mice followed by vehicle or combined RepSox plus SB4 treatment from day ˜31-38 These treatments were followed by anti-PD-1.1 treatment as described above from day 40-53 followed by euthanasia and harvesting tissues for analysis of donor LCMV-specific CD8+ T cell responses. All protocols and procedures followed are approved by the OSU Institutional Animal Care and Use Committee (IACUC) under the IACUC protocol #2019A00000055.

### (8) Mouse B16 Melanoma Tumor Model

Healthy, wild-type 8 week-old C57BL/6 mice were purchased from Jackson Laboratory. B16-F10 tumor cell line stably expressing the GP33 surface glycoprotein and GFP reporter was a kind gift from Dr. Andreas Wieland at the Ohio State University. The cell line was cultured in complete RPMI medium (containing 10% FBS and 1× Pen/Strep) at 37° C. and 5% CO2. On day 0, 250,000 viable B16-F10-LCMV-GP tumor cells were injected subcutaneously into the right flank of each mouse. Tumors became palpable around day 10 and were measured daily via caliper. Beginning on day 10 or 11 (when tumors reached at least 100 mm3), mice received either Vehicle (DMSO in PBS), RepSox (TGFβR1 inhibitor, 5 mg/Kg), SB4 (BMP-4 agonist, 5 mg/Kg) or RepSox+SB4 (each 5 mg/Kg) via i.p. injection every 2 days. Mice were sacrificed on day 17, or when tumors exceeded 1.6 cm.

### (9) PMA Stimulation of Human CBMCs

On days 7, 14, 21, and 28 (and day 35 and 42 for recovery experiments), human CBMCs were stimulated with PMA/Ionomycin (Cell Stimulation Cocktail plus protein transport inhibitors, eBioscience) for 3 hours at 37° C., 5% CO₂. Incubation was followed immediately by antibody intracellular staining for flow cytometry.

### (10) Flow Cytometry Antibody Staining

Single cell suspensions were stained with a comprehensive panel, and data were collected using a Cytek Aurora 4-laser spectral cytometer. Dead cells were stained with Ghost Dye™ Violet 510 (Tonbo Biosciences). Human surface antibodies used for staining throughout this study include: Brilliant Violet™ 421-anti-CD197 (CCR7) (Clone G043H7), Brilliant Violet 605™ anti-CD107a (H4A3), Brilliant Violet 711™ anti-CD274 (B7-H1, PD-L1) (29E.2A3), Brilliant Violet 711™ anti-CD39 (A1), FITC-anti-CD3 (UCHT1), PerCP/Cyanine5.5-anti-CD8 (RPA-T8), Alexa Fluor® 700-anti-CD4 (A161A1), Alexa Fluor® 647 anti-CD11a (HI111), APC-anti-CD45RO (UCHL1), APC-anti-CD101 (BB27), PE-anti-CD279 (PD-1) (EH12.2H7), PE/Dazzle™ 594-anti-LAG3 (11C3C65), PE/Cyanine7-anti-CD324 (E-cadherin) (DECMA-1) (BioLegend), PE/Cyanine5-anti-CD127 (IL-7R) (A019D5) (BioLegend), and APC/Cyanine7-anti-CD103 (Ber-ACT8) (BioLegend). Cells were then fixed and permeabilized for intracellular staining using the Transcription Factor Fixation/Permeabilization kit (Invitrogen). Human intracellular antibodies used for staining throughout this study include Brilliant Violet™ 650-anti-IL-2 (MQ1-17H12), Brilliant Violet™ 785-anti-Tbet (4B10), Pacific Blue-anti-Granzyme B (GB11), APC anti-Perforin (dG9) (Biolegend), V450-anti-IFN-γ (B27). PE-anti-TCF-7/TCF-1 (S33-966) (BD Biosciences), PE/Cyanine7-anti-TNFα (MAb11), and PE/Cyanine7-anti-Ki67 (SolA15) (Invitrogen).

Antibodies used for mouse LCMV infection and mouse P14 co-culture experiments include AlexaFluor® 594 anti-CD107a (LAMP-1) (1D4B), Brilliant Violet™ 421-anti-CD44 (IM7), Brilliant Violet™ 711-anti-Cx3cr11 (SA011F11), Brilliant Violet 605™ anti-CD103 (2E7), FITC-anti-CD90.1 (Thy1.1) (OX-7), PerCP/Cyanine5.5-anti-CD8a (53-6.7), APC/Cyanine7-anti-CD279 (PD-1) (29F.1A12), PE-anti-CD39 (Duha59), PE-anti-Tim3 (RMT3-23) (Biolegend), APC-anti-IFN-γ (XMG1.2) (Biolegend), and PE/Cyanine7-anti-Ki67 (SolA15) (Invitrogen). LCMV glycoprotein 33 (GP33) specific CD8+ T cells were detected by tetramerization of GP33 monomers (NIH Tetramer Core Facility) conjugated to streptavidin-APC (eBioscience).

### (11) Tracking Proliferation of Rested Human CD8+ T Cells

Human CBMCs from ex vivo stimulation experiments (acute or chronic TCR, and chronic TCR+TGFβ1) were labeled with 5(6)-Carboxyfluorescein diacetate N-succinimidyl ester (CFSE) (1 μM, Sigma) by staining in PBS for 7 minutes at room temperature, followed by quenching with 20% FBS. Proliferation was tracked on days 35 and 42 by assessing CFSE fluorescence via flow cytometry. Each peak of CFSE fluorescence signal represents one cell division cycle.

### (12) Cytotoxicity Assay of Human CD8+ T Cells

Acutely or chronically stimulated human CD8+ T cells (on day 28) were used for co-culture with human THP-1 AML or MDA-MB-231 breast carcinoma cell lines to assess CD8+ T cell anti-tumor activity. Tumor cells were labeled with CFSE (1 μM) prior to co-culture. 30,000 viable human CBMCs from each in vitro condition were added per well with 30,000 viable CFSE-labeled tumor cells (or 6,000 and 3,000 CBMCs for 1:5 and 1:10 ratios, respectively), in complete RPMI medium containing 10 ng/ml of IL-15. Cells were treated with 2 μg/ml of human anti-CD3 for THP-1 co-culture experiments, or 10 g/ml of anti-CD3 for MDA-MB-231 co-culture experiments. Co-cultured cells were incubated for 18 hours at 37° C. and 5% CO₂, followed by antibody staining for flow cytometry analysis.

### (13) Bisulfite Conversion and PCR Amplification of Isolated Human DNA

Sorted CD8+ cell pellets were used for DNA isolation and subsequent bisulfite conversion using the EZ DNA Methylation-Direct Kit (Zymo). Bisulfite-converted genomic DNA was used for PCR amplification of a key differentially methylated region (DMR) in the TBX21 locus using the following primers: Forward primer-GGTTAGTGTAGTAAAGTTTGTAGGG (SEQ ID NO: 1), Reverse primer-CCTCTAAAATCCAACATAACCTTCTCC (SEQ ID NO: 2). The amplicon DNA size was confirmed by gel electrophoresis and purified using the Zymoclean Gel DNA Recovery Kit (Zymo).

(14) Sequencing PCR Amplicons with Oxford Nanopore MinION

Purified amplicon DNA was prepared for sequencing by following the Native barcoding amplicons protocol using EXP-NBD104 and SQK-LSK109 sequencing kits (Oxford Nanopore Technologies). DNA sequencing library of the purified PCR amplicons was prepared using the Native Barcoding Kit (EXP-NBD104) and Ligation Sequencing Kit (SQK-LSK109) (OxfordNanopore Technologies). The prepared DNA library was loaded onto an R9 flow cell on the MinION sequencing device (Oxford Nanopore Technologies). The resultant FASTQ files from the MinION sequencing were extracted for downstream analysis of CpG methylation at the amplified DMRs using the NanoEM pipeline.

### (15) RNA-Sequencing

RNA was isolated using PicoPure™ RNA Isolation Kit (Applied Biosystems) from frozen cell pellets of human CD8+ T cells that were FACS-purified from days 0 (naïve), 7, and 28. Following the kit protocol. Partek Flow was used for RNA-seq analysis. In brief, pre-alignment QC/QA analysis was performed on the FASTQ files, followed by read alignment on human whole genome hg38 using STAR. The aligned reads were quantified to the hg38 RefSeq Transcripts 98 using the Quantify to annotation model on Partek and gene counts were filtered (exclude features where maximum <50.0). Filtered gene counts were normalized using median ratio for DESeq2 analysis to measure differential gene expression among different populations of CD8+ T cells. Differentially expressed genes (DEGs) were identified for genes showing fold change ≥2 and p-value 0.05.

### (16) Whole-Genome Bisulfite Sequencing

Human CD8+ T cells were sorted on days 0 (naïve), 7, and 28, and genomic DNA was extracted and bisulfite-treated to convert unmethylated cytosine to uracil residues using the EZ DNA Methylation-Direct Kit (Zymo). DNA libraries were prepared using Swift Accel-NGS® D Methyl-Seq DNA Library Kit according to the manufacturer's protocol. The libraries were purified by the AMPureXP Beads followed by QC using Qubit method, and the purified DNA was captured on an Illumina flow cell for cluster generation. Libraries were sequenced on the NovaSeq-S2 platform following the manufacturer's protocols. Sequencing data were aligned to the Hg38 genome using BSMAP. Adapter were trimmed from FASTQ sequences (trimglore) and mapped to the hg38 genome using the BSMAP v. 2.74 software. CpGs were extracted by the methratio.py in the BSMAP package. Differential analysis of CpG methylation among the datasets was determined with a Bayesian hierarchical model to detect regional methylation differences with at least three CpG site. Differentially methylated regions (DMRs) were identified using Bioconductor package DSS and custom R scripts with a threshold of 30% change in methylation ratio and P-value <0.01 with at least 10 reads per CpG site as a cutoff.

### (17) Enrichment Analysis

Integrative epigenetic-transcriptional analysis was performed using the Ingenuity Pathway Analysis software (Qiagen). IPA was first performed on differentially methylated, accessible, or expressed genes. Then, a multi-comparison analysis was performed to identify the top significant upstream regulators that are common among all listed comparisons with P-value <10⁻⁵ GO non-redundant biological process and over-representation analyses of DEGs among human CD8+ T cell populations were performed using WebGestalt with human genome as a reference set. Gene set enrichment analysis was performed on GSEA software (UC San Diego and Broad Institute) with the default settings and maximum set size of 500. Pathway enrichment analysis was performed on the DEGs or overlapping genes using NCI-Nature pathway database. Enrichment analysis of ChIP-seq TF binding element enrichment analysis was performed on the indicated DMR list or gene list using ChIP-seq atlas pipeline, and significant results were filtered with P-value <0.05. Genomic annotation of the DMR lists was performed using the Genomic Regions Enrichment of Annotations Tool with genomic region annotation to the single nearest gene.

### (18) Statistical Analysis

Statistical analysis was performed using Prism 9 (GraphPad) software, and statistical significance was determined if P-value is less than 0.05. Comparisons were made using Mann-Whitney U test or unpaired t test with Welch's correction (for pairwise comparisons).

### Example 3

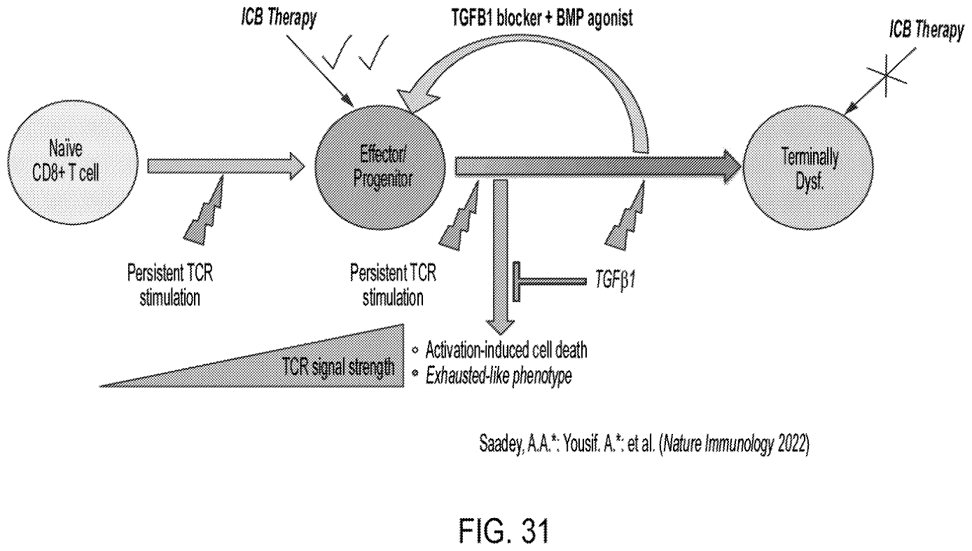

BMP “Bone Morphogenetic Protein” ligands are involved in several tissue biological and developmental processes, such as bone regeneration, vasculature biology, kidney, and brain development. Being members of the TGFβ superfamily, they can signal through SMAD-dependent or SMAD-independent pathways, with preferential activation of SMAD1/5/8 phosphorylation. We have discovered a novel role of BMP signaling in preserving the effector functions and enhancing survival of chronically stimulated human CD8 T cells using a selective BMP4 agonist therapy that stabilizes SMAD1/5/8 phosphorylation. In addition, when combined with TGFβR1 blocker, this therapy significantly enhances exhausted T cell responses to immune checkpoint blockade therapy during chronic viral infection and improves tumor control in animals FIG. 31.

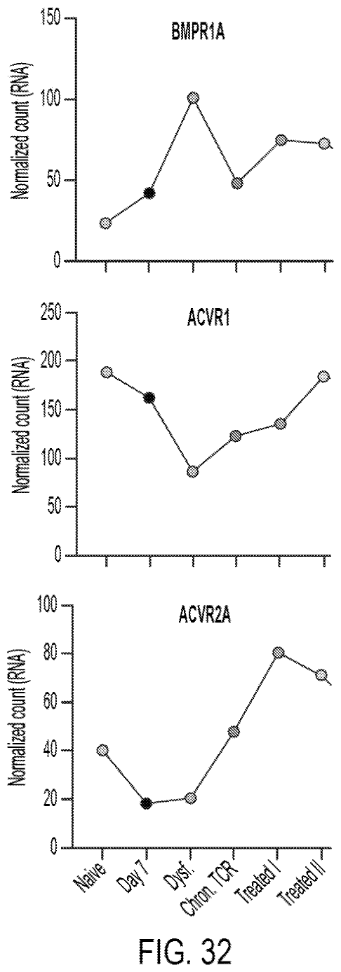

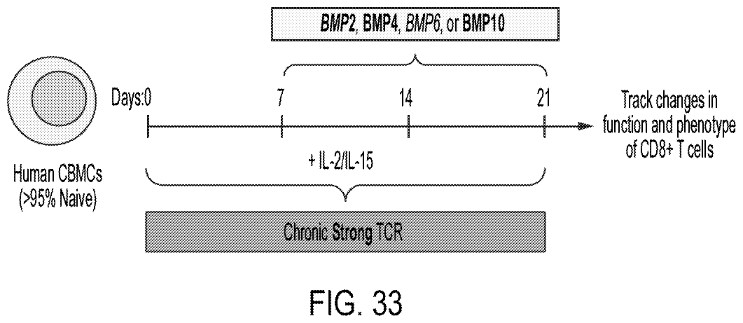

BMP ligands can signal through type 1 (BMPR1, ACVR1) or type 2 receptors (BMPR2, ACVR2), which typically form heterotetramers upon ligand binding. Once heterotetramers are activated by ligands, they phosphorylate SMAD1/5/8 that binds to SMAD4 and translocates to nucleus and bind to target genes. However, different BMP ligands show varying strengths in activating SMAD1/5/8 phosphorylation, with some ligands inducing strong (e.g., BMP2. BMP4. BMP6), and others inducing medium or weak activation of SMAD1/5/8 phosphorylation, such as BMP10, BMP12 and BMP14. Interestingly, our transcriptional profiling analysis show that BMP receptors are differentially expressed on dysfunctional human CD8 T cells. We found that BMPR1A is upregulated on chronically TCR+ TGFβ1 stimulated human CD8+ T cells, while ACVR1 and ACVR2A show increased expression in TGFβR1 blocker-treated human CD8 T cells suggesting differential responses to BMP ligands (FIG. 32). To determine whether and which natural BMP ligand(s) recapitulate BMP4-agonist-mediated effects on dysfunctional CD8 T cells, we treated human CD8 T cells with different BMP ligands, including BMP2, BMP4, BMP6, and BMP10 under chronic strong TCR stimulation (FIG. 33).

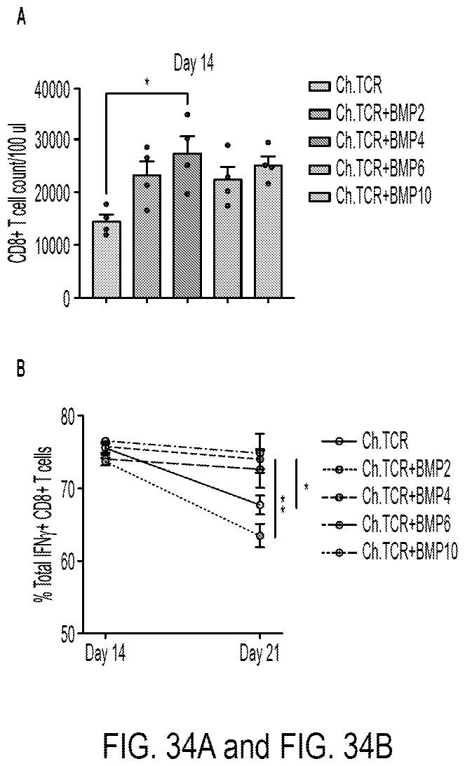

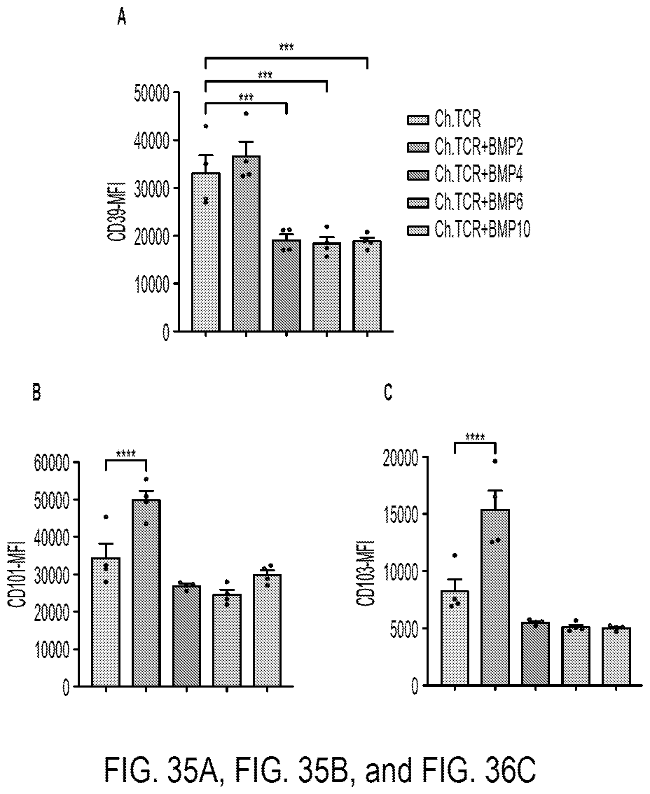

We found that only BMP4 preserved significantly higher numbers of chronically stimulated human CD8 T cells (FIG. 34A). Importantly, both BMP4 and BMP10 maintained effector function of human CD8 T cells, indicated by maintaining significantly higher frequencies of IFNγ-producing CD8 T cells (FIG. 34B). In contrast, BMP2 treatment induced significantly higher expression levels of terminal exhaustion-associated molecules, such as CD39 (FIG. 35A), CD101 (FIG. 35B), and CD103 (FIG. 35C), which were downregulated during BMP4. BMP6 or BMP10 treatment. These data reveal novel differential effects of BMP ligands on human CD8 T cell phenotype and function during chronic stimulation. It further indicates that selective agonists for BMP4, BMP6, and BMP10 ligand can improve the effector function of exhausted CD8 T cells and enhance the efficacy of T cell-based immunotherapies.

### E. REFERENCES

Abdel-Hakeem, M. S. et al. Epigenetic scarring of exhausted T cells hinders memory differentiation upon eliminating chronic antigenic stimulation. Nat. Immunol. 22, 1008-1019 (2021).
Abdelsamed. H. A. et al. Beta cell-specific CD8+ T cells maintain stem cell memory-associated epigenetic programs during type 1 diabetes. Nat. Immunol. 21, 578-587 (2020).
Abdelsamed, H. A. et al. Human memory CD8 T cell effector potential is epigenetically preserved during in vivo homeostasis. J Exp. Med. 214, 1593-1606 (2017).
Abdelsamed, H. Z., C.; Nguyen, H.; Rutishauser, R. L.; Fan, Y.; Ghoneim, H. E.; et al. Beta cell-specific CD8 T cells acquire stem-cell memory-associated epigenetic programs during development of type 1 diabetes. Nature Immunology (In Press).
Ahmadzadeh, M. & Rosenberg, S. A. TGF-beta 1 attenuates the acquisition and expression of effector function by tumor antigen-specific human memory CD8 T cells. J Immunol 174, 5215-5223, doi:10.4049/jimmunol.174.9.5215 (2005).
Akondv, R. S. et al. Origin and differentiation of human memory CD8 T cells after vaccination. Nature 552, 362-367 (2017).
Alfei, F. et al. TOX reinforces the phenotype and longevity of exhausted T cells in chronic viral infection. Nature 571, 265-269, doi:10.1038/s41586-019-1326-9 (2019).
Angelosanto, J. M., Blackburn, S. D., Crawford, A. & Wherry, E. J. Progressive loss of memory T cell potential and commitment to exhaustion during chronic viral infection. J Virol 86, 8161-8170, doi:10.1128/JVI.00889-12 (2012).
Angelosanto, J. M., Blackburn, S. D., Crawford, A. & Wherry, E. J. Progressive loss of memory T cell potential and commitment to exhaustion during chronic viral infection. J. Virol. 86, 8161-8170 (2012).
Batlle, E. & Massague, J. Transforming Growth Factor-beta Signaling in Immunity and Cancer. Immunity 50, 924-940, doi:10.1016/j.immuni.2019.03.024 (2019).
Begley, C. G. & Ellis, L. M. Drug development: Raise standards for preclinical cancer research. Nature 483, 531-533, doi:10.1038/483531a (2012).
Beltra, J.-C. et al. Developmental Relationships of Four Exhausted CD8+ T Cell Subsets Reveals Underlying Transcriptional and Epigenetic Landscape Control Mechanisms. Immunity 52, 825-841.e8 (2020).
Bintu, L. et al. Dynamics of epigenetic regulation at the single-cell level. Science 351, 720-724, doi:10.1126/science.aab2956 (2016).
Bird, A. DNA methylation patterns and epigenetic memory. Genes Dev 16, 6-21, doi: 10.1101/gad.947102 (2002).
Blank, C. U. et al. Defining ‘T cell exhaustion’. Nat Rev Immunol, doi:10.1038/s41577-019-0221-9 (2019).
Bollard, C. M. et al. Tumor-Specific T-Cells Engineered to Overcome Tumor Immune Evasion Induce Clinical Responses in Patients With Relapsed Hodgkin Lymphoma. J Clin Oncol 36, 1128-1139, doi:10.1200/JCO.2017.74.3179 (2018).
Boussiotis, V. A. Molecular and Biochemical Aspects of the PD-1 Checkpoint Pathway. N Engl J Med 375, 1767-1778, doi:10.1056/NEJMra1514296 (2016).
Brunetti. L., Gundry. M. C. & Goodell, M. A. DNMT3A in Leukemia. Cold Spring Harb Perspect Med 7, doi:10.1101/cshperspect.a030320 (2017).
Chen, C.-H. et al. Transforming growth factor beta blocks Tec kinase phosphorylation, Ca2+ influx, and NFATc translocation causing inhibition of T cell differentiation. J. Exp. Med. 197, 1689-1699 (2003).
Chen, Y. et al. BATF regulates progenitor to cytolytic effector CD8+ T cell transition during chronic viral infection. Nat. Immunol. 22, 996-1007 (2021).
Chou, C. K. et al. Cell-intrinsic abrogation of TGF-β signaling delays but does not 984 prevent dysfunction of self/tumor-specific CD8 T cells in a murine model of autochthonous 985 prostate cancer. J. Immunol. Baltim. Md 1950 189, 3936-3946 (2012).
Collier, J. L., Weiss, S. A., Pauken, K. E., Sen, D. R. & Sharpe, A. H. Not-so-opposite ends of the spectrum: CD8+ T cell dysfunction across chronic infection, cancer and autoimmunity. Nat. Immunol. 22, 809-819 (2021).
Collins, P. L. et al. Gene Regulatory Programs Conferring Phenotypic Identities to Human NK Cells. Cell 176, 348-360 e312, doi:10.1016/j.cell.2018.11.045 (2019).
David, C. J. & Massague, J. Contextual determinants of TGFβ action in development, immunity and cancer. Nat. Rev. Mol. Cell Biol. 19, 419-435 (2018).
Donkor, M. K. et al. T cell surveillance of oncogene-induced prostate cancer is impeded by T cell-derived TGF-beta1 cytokine. Immunity 35, 123-134, doi:10.1016/j.immuni.2011.04.019 (2011).
Duhen, T. et al. Co-expression of CD39 and CD103 identifies tumor-reactive CD8 T cells in human solid tumors. Nat. Commun. 9, 2724 (2018).
Flavell, R. A., Sanjabi, S., Wrzesinski, S. H. & Licona-Limón, P. The polarization of immune cells in the tumour environment by TGFbeta. Nat. Rev. Immunol. 10, 554-567 (2010).
Gabriel, S. S. et al. Transforming growth factor-β-regulated mTOR activity preserves 981 cellular metabolism to maintain long-term T cell responses in chronic infection. Immunity 54, 982 1698-1714.e5 (2021).
Gate, D. et al. T-cell TGF-beta signaling abrogation restricts medulloblastoma progression. Proc Natl Acad Sci USA 111, E3458-3466, doi:10.1073/pnas.1412489111 (2014).
Ghoneim, H. E. et al. De Novo Epigenetic Programs Inhibit PD-1 Blockade-Mediated T Cell Rejuvenation. Cell 170, 142-157.e19 (2017).
Ghoneim, H. E., Zamora, A. E., Thomas, P. G. & Youngblood. B. A. Cell-Intrinsic Barriers of T Cell-Based Immunotherapy. Trends Mol Med 22, 1000-1011. doi:10.1016/j.molmed.2016.10.002 (2016).
Goldman, D. C. et al. BMP4 regulates the hematopoietic stem cell niche. Blood 114, 4393-4401 (2009).
Good, C. R. et al. An NK-like CAR T cell transition in CAR T cell dysfunction. Cell 184, 6081-6100.e26 (2021).
Gorelik, L. & Flavell, R. A. Immune-mediated eradication of tumors through the blockade of transforming growth factor-beta signaling in T cells. Nat. Med. 7, 1118-1122 (2001).
Gualandris, A. et al. Role of TGFβ1 and WNT6 in FGF2 and BMP4-driven endothelial differentiation of murine embryonic stem cells. Angiogenesis 25, 113-128 (2022).
Gunderson, A. J. et al. TGFβ suppresses CD8+ T cell expression of CXCR3 and tumor trafficking. Nat. Commun. 11, 1749 (2020).
Hashimoto, M. et al. CD8 T Cell Exhaustion in Chronic Infection and Cancer: Opportunities for Interventions. Annu Rev Med 69, 301-318, doi:10.1146/annurev-med-012017-043208 (2018).
Heczey, A. et al. CAR T Cells Administered in Combination with Lymphodepletion and PD-1 Inhibition to Patients with Neuroblastoma. Mol Ther 25, 2214-2224, doi:10.1016/j.ymthe.2017.05.012 (2017).
Henning, A. N., Roychoudhuri, R. & Restifo, N. P. Epigenetic control of CD8+ T cell differentiation. Nat. Rev. Immunol. 18, 340-356 (2018).
Hensel, N. et al. Memory-like HCV-specific CD8+ T cells retain a molecular scar after cure of chronic HCV infection. Nat. Immunol. 22, 229-239 (2021).
Hore, T. A. et al. Retinol and ascorbate drive erasure of epigenetic memory and enhance reprogramming to naïve pluripotency by complementary mechanisms. Proc. Natl. Acad. Sci. U.S.A. 113, 12202-12207 (2016).
Hudson, W. H. et al. Proliferating Transitory T Cells with an Effector-like Transcriptional Signature Emerge from PD-1+ Stem-like CD8+ T Cells during Chronic Infection. Immunity 51, 1043-1058.e4 (2019).
Janknecht, R., Wells, N. J. & Hunter, T. TGF-beta-stimulated cooperation of smad proteins with the coactivators CBP/p300. Genes Dev 12, 2114-2119, doi:10.1101/gad.12.14.2114 (1998).
Jeong, S. et al. BMP4 and perivascular cells promote hematopoietic differentiation of human pluripotent stem cells in a differentiation stage-specific manner. Exp. Mol. Med. 52, 56-65 (2020).
Kalbasi, A. & Ribas, A. Tumour-intrinsic resistance to immune checkpoint blockade. Nat. 976 Rev. Immunol. 20, 25-39 (2020).
Kalluri, R. & Weinberg, R. A. The basics of epithelial-mesenchymal transition. J. Clin. 974 Invest. 119, 1420-1428 (2009).
Khan, O. et al. TOX transcriptionally and epigenetically programs CD8(+) T cell exhaustion. Nature 571, 211-218, doi:10.1038/s41586-019-1325-x (2019).
Kurachi, M. et al. Optimized retroviral transduction of mouse T cells for in vivo assessment of gene function. Nat Protoc 12, 1980-1998, doi:10.1038/nprot.2017.083 (2017).
Kwoczek, J. et al. Cord blood-derived T cells allow the generation of a more naïve tumor-reactive cytotoxic T-cell phenotype. Transfusion (Paris) 58, 88-99 (2018).
Li, Y. & Kurlander, R. J. Comparison of anti-CD3 and anti-CD28-coated beads with soluble anti-CD3 for expanding human T cells: differing impact on CD8 T cell phenotype and responsiveness to restimulation. J. Transl. Med. 8, 104 (2010).
Liao, Y., Wang, J., Jaehnig, E. J., Shi, Z. & Zhang, B. WebGestalt 2019: gene set analysis toolkit with revamped UIs and APIs. Nucleic Acids Res. 47, W199-W205 (2019).
Lohr, J. et al. Effector T-cell infiltration positively impacts survival of glioblastoma patients and is impaired by tumor-derived TGF-beta. Clin Cancer Res 17, 4296-4308, doi:10.1158/1078-0432.CCR-10-2557 (2011).
Long, A. H. et al. 4-1BB costimulation ameliorates T cell exhaustion induced by tonic signaling of chimeric antigen receptors. Nat Med 21, 581-590, doi:10.1038/nm.3838 (2015).
Mamonkin, M., Puppi. M. & Lacorazza, H. D. Transcription factor ELF4 promotes development and function of memory CD8(+) T cells in Listeria monocytogenes infection. Eur. J. Immunol. 44, 715-727 (2014).
Mann, T. H. & Kaech, S. M. Tick-TOX, it's time for T cell exhaustion. Nat Immunol 20, 1092-1094, doi:10.1038/s41590-019-0478-y (2019).
Mariathasan, S. et al. TGFβ attenuates tumour response to PD-L1 blockade by contributing to exclusion of T cells. Nature 554, 544-548 (2018).
Martin, C. J. et al. Selective inhibition of TGFβ1 activation overcomes primary resistance to checkpoint blockade therapy by altering tumor immune landscape. Sci. Transl. Med. 12, eaay8456 (2020).
Matloubian, M., Concepcion, R. J. & Ahmed, R. CD4+ T cells are required to sustain CD8+ cytotoxic T-cell responses during chronic viral infection. J. Virol. 68, 8056-8063 (1994).
Mayle, A. et al. Dnmt3a loss predisposes murine hematopoietic stem cells to malignant transformation. Blood 125, 629-638, doi:10.1182/blood-2014-08-594648 (2015).
McKarns, S. C. & Schwartz, R. H. Distinct effects of TGF-beta 1 on CD4+ and CD8+ T cell survival, division, and IL-2 production: a role for T cell intrinsic Smad3. J Immunol 174, 2071-2083, doi:10.4049/jimmunol.174.4.2071 (2005).
McLane, L. M., Abdel-Hakeem. M. S. & Wherry. E. J. CD8 T Cell Exhaustion During Chronic Viral Infection and Cancer. Annu. Rev. Immunol. 37, 457-495 (2019).
McLean, C. Y. et al. GREAT improves functional interpretation of cis-regulatory regions. Nat. Biotechnol. 28, 495-501 (2010).
Miller, B. C. et al. Subsets of exhausted CD8+ T cells differentially mediate tumor control and respond to checkpoint blockade. Nat. Immunol. 20, 326-336 (2019).
Moller, S. H., Hsueh, P.-C., Yu, Y.-R., Zhang, L. & Ho, P.-C. Metabolic programs tailor T cell immunity in viral infection, cancer, and aging. Cell Metab. 34, 378-395 (2022).
Moreau, J. M., Velegraki, M., Bolyard, C., Rosenblum, M. D. & Li, Z. Transforming growth factor-β1 in regulatory T cell biology. Sci. Immunol. 7, eabi4613 (2022).
Mouillesseaux. K. P. et al. Notch regulates BMP responsiveness and lateral branching in vessel networks via SMAD6. Nat. Commun. 7, 13247 (2016).
Muroyama, Y. & Wherry, E. J. Memory T-Cell Heterogeneity and Terminology. Cold Spring Harb. Perspect. Biol. 13, a037929 (2021).
Nakashima, H. et al. Modeling tumor immunity of mouse glioblastoma by exhausted CD8+ T cells. Sci. Rep. 8, 208 (2018).
Nolz, J. C. & Harty, J. T. Protective capacity of memory CD8+ T cells is dictated by antigen exposure history and nature of the infection. Immunity 34, 781-793 (2011).
O'Connell. P. et al. SLAMF7 Signaling Reprograms T Cells toward Exhaustion in the Tumor Microenvironment. J. Immunol. Baltim. Md 1950 206, 193-205 (2021).
Oki, S. et al. ChIP-Atlas: a data-mining suite powered by full integration of public ChIP-seq data. EMBO Rep. 19, e46255 (2018).
Page, N. et al. Persistence of self-reactive CD8+ T cells in the CNS requires TOX-dependent chromatin remodeling. Nat. Commun. 12, 1009 (2021).
Park, B. V. et al. TGFbeta1-Mediated SMAD3 Enhances PD-1 Expression on Antigen-Specific T Cells in Cancer. Cancer Discov 6, 1366-1381, doi:10.1158/2159-8290.CD-15-1347 (2016).
Pauken, K. E. et al. Epigenetic stability of exhausted T cells limits durability of reinvigoration by PD-1 blockade. Science 354, 1160-1165 (2016).
Pauken, K. E. et al. Single-cell analyses identify circulating anti-tumor CD8 T cells and markers for their enrichment. J. Exp. Med. 218, e20200920 (2021).
Philip, M. et al. Chromatin states define tumour-specific T cell dysfunction and 972 reprogramming. Nature 545, 452-456 (2017).
Ponomarev, L. C., Ksiazkiewicz, J., Staring, M. W., Luttun, A. & Zwijsen, A. The BMP Pathway in Blood Vessel and Lymphatic Vessel Biology. Int. J. Mol. Sci. 22, 6364 (2021).
Rachidi, S. et al. Platelets subvert T cell immunity against cancer via GARP-TGFβ axis. Sci. Immunol. 2, eaai7911 (2017).
Raju, S. et al. Identification of a T-bethi Quiescent Exhausted CD8 T Cell Subpopulation That Can Differentiate into TIM3+CX3CR1+ Effectors and Memory-like Cells. J. Immunol. Baltim. Md 1950 206, 2924-2936 (2021).
Saadey, A. A., Yousif. A. & Ghoneim, H. E. Chapter Seven—Epigenetic programming of the immune responses in cancer, in Cancer Immunology and Immunotherapy (eds. Amiji, M. M. & Milane, L. S.) 197-235 (Academic Press, 2022). doi:10.1016/B978-0-12-823397-9.00007-7.
Saini, A., Ghoneim, H. E., Lio, C.-W. J., Collins, P. L. & Oltz, E. M. Gene Regulatory Circuits in Innate and Adaptive Immune Cells. Annu. Rev. Immunol. (2022) doi:10.1146/annurev-immunol-101320-025949.
Sakamoto, Y. et al. Long-read whole-genome methylation patterning using enzymatic base conversion and nanopore sequencing. Nucleic Acids Res. 49, e81 (2021).
Satpathy. A. T. et al Massively parallel single-cell chromatin landscapes of human 969 immune cell development and intratumoral T cell exhaustion. Nat. Biotechnol. 37, 925-936 970 (2019).
Scharer, C. D., Bally, A. P., Gandham, B. & Boss, J. M. Cutting Edge: Chromatin Accessibility Programs CD8 T Cell Memory. J Immunol 198, 2238-2243, doi:10.4049/jimmunol.1602086 (2017).
Scharer, C. D., Barwick, B. G., Youngblood, B. A., Ahmed, R. & Boss, J. M. Global DNA methylation remodeling accompanies CD8 T cell effector function. J Immunol. Baltim. Md 1950 191, 3419-3429 (2013).
Scott, A. C. et al. TOX is a critical regulator of tumour-specific T cell differentiation. Nature 571, 270-274, doi:10.1038/s41586-019-1324-y (2019).
Scott-Browne, J. P. et al. Dynamic Changes in Chromatin Accessibility Occur in CD8(+) T Cells Responding to Viral Infection. Immunity 45, 1327-1340, doi:10.1016/j.immuni.2016.10.028 (2016).
Sen, D. R. et al. The epigenetic landscape of T cell exhaustion. Science 354, 1165-1169, doi:10.1126/science.aae0491 (2016).
Seo, H. et al. TOX and TOX2 transcription factors cooperate with NR4A transcription factors to impose CD8(+) T cell exhaustion. Proc Natl Acad Sci USA 116, 12410-12415, doi:10.1073/pnas.1905675116 (2019).
Shah, T. A. & Rogers, M. B. Unanswered Questions Regarding Sex and BMP/TGF-beta Signaling. J Dev Biol 6, doi:10.3390/jdb6020014 (2018).
Shakiba, M. et al. TCR signal strength defines distinct mechanisms of T cell dysfunction and cancer evasion. J. Exp. Med. 219, e20201966 (2022).
Sharma, P. & Allison, J. P. Dissecting the mechanisms of immune checkpoint therapy. Nat. Rev. Immunol. 20, 75-76 (2020).
Sharma, P. & Allison, J. P. The future of immune checkpoint therapy. Science 348, 56-61, doi:10.1126/science.aaa8172 (2015).
Tauriello, D. V. F. et al. TGFβ drives immune evasion in genetically reconstituted colon cancer metastasis. Nature 554, 538-543 (2018).
Tauriello, D. V. F., Sancho, E. & Batlle, E. Overcoming TGFβ-mediated immune evasion in cancer. Nat. Rev. Cancer 22, 25-44 (2022).
Thaker, Y. R., Raab, M., Strebhardt, K. & Rudd, C. E. GTPase-activating protein Rasal1 associates with ZAP-70 of the TCR and negatively regulates T-cell tumor immunity. Nat. Commun. 10, 4804 (2019).
Thomas, D. A. & Massague, J. TGF-beta directly targets cytotoxic T cell functions during tumor evasion of immune surveillance. Cancer Cell 8, 369-380, doi:10.1016/j.ccr.2005.10.012 (2005).
Tinoco, R., Alcalde, V., Yang, Y., Sauer, K. & Zuniga, E. I. Cell-intrinsic transforming 978 growth factor-beta signaling mediates virus-specific CD8+ T cell deletion and viral 979 persistence in vivo. Immunity 31, 145-157 (2009).
Tonnerre, P. et al. Differentiation of exhausted CD8+ T cells after termination of chronic antigen stimulation stops short of achieving functional T cell memory. Nat. Immunol. 22, 1030-1041 (2021).
Utzschneider, D. T. et al. T cells maintain an exhausted phenotype after antigen withdrawal and population reexpansion. Nat Immunol 14, 603-610, doi:10.1038/ni.2606 (2013).
van der Leun, A. M., Thommen. D. S. & Schumacher, T. N. CD8+ T cell states in human cancer: insights from single-cell analysis. Nat. Rev. Cancer 20, 218-232 (2020).
Wang, Y. et al. Single-molecule long-read sequencing reveals the chromatin basis of gene expression. Genome Res 29, 1329-1342. doi: 10.1101/gr.251116.119 (2019).
West, E. E. et al. Tight regulation of memory CD8(+) T cells limits their effectiveness during sustained high viral load. Immunity 35, 285-298 (2011).
Woehrle, T. et al. Pannexin-1 hemichannel-mediated ATP release together with P2X1 and P2X4 receptors regulate T-cell activation at the immune synapse. Blood 116, 3475-3484 (2010).
Woroniecka. K. et al. T-Cell Exhaustion Signatures Vary with Tumor Type and Are Severe in Glioblastoma. Clin Cancer Res 24, 4175-4186, doi: 10.1158/1078-0432.CCR-17-1846 (2018).
Wu, H. et al. Detection of differentially methylated regions from whole-genome bisulfite sequencing data without replicates. Nucleic Acids Res. 43, e141 (2015).
Wu, X. & Zhang, Y. TET-mediated active DNA demethylation: mechanism, function and beyond. Nat Rev Genet 18, 517-534, doi:10.1038/nrg.2017.33 (2017).
Xi, Y. & Li, W. BSMAP: whole genome bisulfite sequence MAPping program. BMC Bioinformatics 10, 232 (2009).
Yamada, T., Park, C. S., Mamonkin, M. & Lacorazza, D. The transcription factor ELF4 controls proliferation and homing of CD8+ T cells via the Krippel-like factors KLF4 and KLF2. Nat. Immunol. 10, 618-626 (2009).
Yang, Y. et al. TCR engagement negatively affects CD8 but not CD4 CAR T cell expansion and leukemic clearance. Sci Transl Med 9, doi:10.1126/scitranslmed.aag1209 (2017).
Yao, C. et al. Single-cell RNA-seq reveals TOX as a key regulator of CD8(+) T cell persistence in chronic infection. Nat Immunol 20, 890-901, doi:10.1038/s41590-019-0403-4 (2019).
Yates, K. B. et al. Epigenetic scars of CD8+ T cell exhaustion persist after cure of chronic infection in humans. Nat. Immunol. 22, 1020-1029 (2021).
Yoon, J. H. et al. Activin receptor-like kinase5 inhibition suppresses mouse melanoma by ubiquitin degradation of Smad4, thereby derepressing eomesodermin in cytotoxic T lymphocytes. EMBO Mol Med 5, 1720-1739, doi:10.1002/emmm.201302524 (2013).
Youngblood. B. et al. Effector CD8 T cells dedifferentiate into long-lived memory cells. Nature 5552, 404-409 (2017).
Yousif. A. & Ghoneim, H. E. T cell exhaustion—a memory locked behind scars. Nat. Immunol. 22, 938-940 (2021).
Zheng, L. et al. Pan-cancer single-cell landscape of tumor-infiltrating T cells. Science 374, abe6474 (2021).

1. A method of rescuing the functional phenotype of exhausted T cells in a subject in need thereof comprising administering to the subject a 1) transforming growth factor-3 receptor 1 (TGFβR1) inhibitor and 2) a bone morphogenic protein 4 (BMP4), BMP6, or BMP10 protein, and/or a BMP4, BMP6, or BMP10 agonist.

2. The method of rescuing the functional phenotype of exhausted T cells of claim 1, wherein the TGFβR1 inhibitor comprises RepSox, SB525334, GW788388, Vactosertib, SD-208, Galunisertib, and/or LY3200882.

3. The method of rescuing the functional phenotype of exhausted T cells of claim 1, wherein the BMP4 agonist comprises SB4, SJ000063181, SJ000291942, and/or SJ000370178.

4. The method of rescuing the functional phenotype of exhausted T cells of claim 1, further comprising administering to the subject vitamin C.

5. A method of rescuing the functional phenotype of exhausted T cells in a subject in need thereof comprising administering to the subject a vector encoding clustered regularly interspaced short palindromic repeat (CRISPR)/CRISPR-associated 9 (Cas9) endonuclease integration system wherein the Cas9 endonuclease complexed with a guide RNA (gRNA) that targets TGFβR1 gene; and wherein expression of the CRISPR/Cas9 endonuclease integration systems excises all or a functional fragment of the TGFβR1.

6. (canceled)

7. The method of rescuing the functional phenotype of exhausted T cells of claim 5, wherein the expression of the Cas9 endonuclease is operatively linked to a T cell specific promoter.

8. The method of rescuing the functional phenotype of exhausted T cells of claim 5, wherein the expression of the Cas9 endonuclease is operatively linked to an inducible or constitutive promoter.

9. A cancer or infectious disease treatment regimen comprising 1) one or more checkpoint inhibitors, 2) one or more transforming growth factor-β receptor 1 (TGFβR1) inhibitors and 3) one or more bone morphogenic protein 4 (BMP4), BMP6, or BMP10 protein, and/or a BMP4, BMP6, or BMP10 agonists.

10. The cancer or infectious disease treatment regimen of claim 9, wherein the immune checkpoint inhibitor targets PD-1, PD-L1, PD-L2, CTLA-4, LAG-3, IDO, B7-H3, B7-H4, T cell immunoreceptor with Ig and ITIM domains (TIGIT), CD96, B- and T-lymphocyte attenuator (BTLA), V-domain Ig suppressor of T cell activation (VISTA), or TIM-3.

11-19. (canceled)

20. The cancer or infectious disease treatment regimen of claim 9, wherein the TGFβR1 inhibitor comprises RepSox, SB525334, GW788388, Vactosertib, SD-208, Galunisertib, and/or LY3200882.

21. The cancer or infectious disease treatment regimen of claim 9, wherein the BMP4 agonist comprises SB4, SJ000063181, SJ000291942, and/or SJ000370178.

22. The cancer or infectious disease treatment regimen of claim 9, further comprising an antioxidant.

23. The cancer or infectious disease treatment regimen of claim 22, wherein the antioxidant comprises vitamin C.

24. A method of treating a cancer or an infectious disease in a subject comprising administering to the subject the treatment regimen of claim 9.

25-42. (canceled)

43. A method of increasing the efficacy of or reducing resistance to an immune checkpoint blockade in a subject receiving treatment with an immune checkpoint inhibitor comprising administering to the subject 1) one or more transforming growth factor-β receptor 1 (TGFβR1) inhibitors or a vector encoding a CRISPR/Cas9 endonuclease integration system comprising a guide RNA (gRNA) that targets TGFβR1 gene and 2) one or more bone morphogenic protein 4 (BMP4), BMP6, or BMP10 protein, and/or a BMP4, BMP6, or BMP10 agonists.

44. The method of increasing the efficacy of or reducing resistance to an immune checkpoint blockade of claim 43, wherein the immune checkpoint inhibitor targets PD-1, PD-L1, PD-L2, CTLA-4, LAG-3, IDO, B7-H3, B7-H4, T cell immunoreceptor with Ig and ITIM domains (TIGIT), CD96, B- and T-lymphocyte attenuator (BTLA), V-domain Ig suppressor of T cell activation (VISTA), or TIM-3.

45-53. (canceled)

54. The method of treating a cancer or an infectious disease of any of claim 43, wherein the TGFβR1 inhibitor comprises RepSox, SB525334, GW788388, Vactosertib, SD-208, Galunisertib, and/or LY320082.

55. The method of treating a cancer or an infectious disease of any of claim 43, wherein the BMP4 agonist comprises SB4, SJ000063181, SJ000291942, and/or SJ000370178.


SEQUENCE LISTING (2 sequences)

SEQ ID NO: 1 - DNA, 25 bp, synthetic construct
  source 1..25: mol_type = other DNA
ggttagtgtagtaaagtttgtaggg

SEQ ID NO: 2 - DNA, 27 bp, synthetic construct
  source 1..27: mol_type = other DNA
cctctaaaatccaacataaccttctcc